(19) United States
(12) Patent Application Publication
(10) Pub. No.: US 2020/0038420 A1
(43) Pub. Date: Feb. 6, 2020

(54) AMINOSTEROL COMPOSITIONS AND METHODS OF USING THE SAME FOR TREATING DEPRESSION

(71) Applicant: Enterin, Inc., Philadelphia, PA (US)

(72) Inventors: Denise Barbut, Philadelphia, PA (US); Michael Zasloff, Philadelphia, PA (US)

(73) Assignee: Enterin, Inc., Philadelphia, PA (US)

(21) Appl. No.: 16/530,295

(22) Filed: Aug. 2, 2019

Related U.S. Application Data
(60) Provisional application No. 62/714,468, filed on Aug. 3, 2018.
(60) Provisional application No. 62/714,470, filed on Aug. 3, 2018.
(60) Provisional application No. 62/789,481, filed on Jan. 7, 2019.

(51) Int. Cl. A61K 31/575; A61K 9/00; A61P 25/24

(52) U.S. Cl. CPC A61K 31/575; A61P 25/24; A61K 9/0053; A61K 9/0043

(57) ABSTRACT

The present application relates generally to methods for treating, preventing, and/or slowing the onset or progression of depression and a variety of symptoms related thereto with aminosterols or pharmaceutically acceptable salts or derivatives thereof.

### CROSS-REFERENCE TO RELATED APPLICATIONS

This application claims the priority benefits under 35 USC § 119 to U.S. provisional Application No. 62/714,470, filed Aug. 3, 2018; U.S. provisional Application No. 62/714,468, filed Aug. 3, 2018; and U.S. provisional Application No. 62/789,481, filed Jan. 7, 2019, the entire contents of which are incorporated herein by reference in their entirety FIELD

The present application relates generally to compositions and methods for treating, preventing, and/or slowing the onset or progression of depression and/or a related symptom, and disorders related thereto, with aminosterols or pharmaceutically acceptable salts or derivatives thereof.

### BACKGROUND

Aminosterols are amino derivatives of a sterol. Examples of aminosterols include squalamine and Aminosterol 1436 (also known as trodusquemine and MSI-1436).

Squalamine is a unique compound with a structure of a bile acid coupled to a polyamine (spermidine):

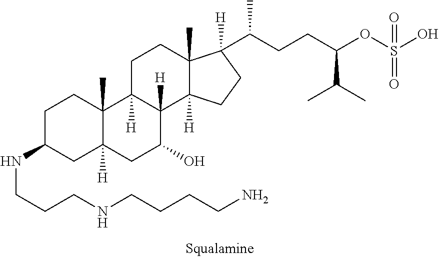

The discovery of squalamine, the structure of which is shown above, was reported by Michael Zasloff in 1993 (U.S. Pat. No. 5,192,756). Squalamine was discovered in various tissues of the dogfish shark (Squalus acanthias) in a search for antibacterial agents. The most abundant source of squalamine is in the livers of Squalus acanthias, although it is found in other sources, such as lampreys (Yun et al., 2007).

Several clinical trials have been conducted relating to the use of squalamine, including the following:

(1) ClinicalTrials.gov Identifier NCT01769183 for “Squalamine for the Treatment in Proliferative Diabetic Retinopathy,” by Elman Retina Group (6 participants; study completed August 2014);

(2) ClinicalTrials.gov Identifier NCT02727881 for “Efficacy and Safety Study of Squalamine Ophthalmic Solution in Subjects With Neovascular AMD (MAKO),” by Ohr Pharmaceutical Inc. (230 participants; study completed December 2017);

(3) ClinicalTrials.gov Identifier NCT02614937 for “Study of Squalamine Lactate for the Treatment of Macular Edema Related to Retinal Vein Occlusion,” by Ohr Pharmaceutical Inc. (20 participants; study completed December 2014);

(4) ClinicalTrials.gov Identifier NCT01678963 for “Efficacy and Safety of Squalamine Lactate Eye Drops in Subjects With Neovascular (Wet) Age-related Macular Degeneration (AMD),” by Ohr Pharmaceutical Inc. (142 participants; study completed March 2015);

(5) ClinicalTrials.gov Identifier NCT00333476 for “A Study of MSI-1256F (Squalamine Lactate) To Treat “Wet” Age-Related Macular Degeneration,” by Genaera Corporation (140 participants; study terminated);

(6) ClinicalTrials.gov Identifier NCT00094120 for “MSI-1256F (Squalamine Lactate) in Combination With Verteporfin in Patients With “Wet” Age-Related Macular Degeneration (AMD),” by Genaera Corporation (60 participants; study completed February 2007);

(7) ClinicalTrials.gov Identifier NCT00089830 for “A Safety and Efficacy Study of MSI-1256F (Squalamine Lactate) To Treat “Wet” Age-Related Macular Degeneration,” by Genaera Corporation (120 participants; study completed May 2007); and

(8) ClinicalTrials.gov Identifier NCT03047629 for Evaluation of Safety and Tolerability of ENT-01 for the Treatment of Parkinson's Disease Related Constipation (RASMET) (50 participants; study completed Jun. 14, 2018).

Aminosterol 1436 is an aminosterol isolated from the dogfish shark, which is structurally related to squalamine (U.S. Pat. No. 5,840,936). It is also known as MSI-1436, trodusquemine and produlestan.

Several clinical trials have been conducted relating to the use of Aminosterol 1436:

(1) ClinicalTrials.gov Identifier NCT00509132 for “A Phase I, Double-Blind, Randomized, Placebo-Controlled Ascending IV Single-Dose Tolerance and Pharmacokinetic Study of Trodusquemine in Healthy Volunteers,” by Genaera Corp.;

(2) ClinicalTrials.gov Identifier NCT00606112 for “A Single Dose, Tolerance and Pharmacokinetic Study in Obese or Overweight Type 2 Diabetic Volunteer,” by Genaera Corp.;

(3) ClinicalTrials.gov Identifier NCT00806338 for “An Ascending Multi-Dose, Tolerance and Pharmacokinetic Study in Obese or Overweight Type 2 Diabetic Volunteers,” by Genaera Corp.; and

(4) ClinicalTrials.gov Identifier: NCT02524951 for “Safety and Tolerability of MSI-1436C in Metastatic Breast Cancer,” by DepyMed Inc.

Depression is a state of low mood and aversion to activity, that can affect a person's thoughts, behavior, tendencies, feelings, and sense of well-being. Depression is often accompanied by low self-esteem, loss of interest in normally enjoyable activities, low energy, and pain without a clear cause. Major depressive disorder (MDD), also known simply as depression, is a mental disorder characterized by at least two weeks of low mood that is present across most situations. Some people have periods of depression separated by years in which they are normal, while others nearly always have symptoms present. Major depressive disorder can negatively affect a person's personal life, work life, or education, as well as sleeping, eating habits, and general health. Between 2-8% of adults with major depression die by suicide, and about 50% of people who die by suicide had depression or another mood disorder.

Major depressive disorder affected approximately 216 million people (3% of the world's population) in 2015. The percentage of people who are affected at one point in their life varies from 7% in Japan to 21% in France. Lifetime rates are higher in the developed world (15%) compared to the developing world (11%). It causes the second most years lived with disability, after lower back pain.

The three most common treatments for depression are psychotherapy, medication, and electroconvulsive therapy. The full potential of aminosterols for use in treatment has yet to be determined.

### SUMMARY

The present application relates generally to compositions and methods for treating, preventing, and/or slowing the onset of depression and/or a related symptom. The methods comprise administering at least one aminosterol or pharmaceutically acceptable salt or derivative thereof to a subject in need. This disclosure also describes the determination and administration of a “fixed aminosterol dose” that is not age, size, or weight dependent but rather is individually calibrated.

The aminosterol or a salt or derivative thereof can be formulated with one or more pharmaceutically acceptable carriers or excipients. Preferably the aminosterol is a pharmaceutically acceptable grade of the aminosterol.

In one embodiment, the invention encompasses a method of treating, preventing and/or slowing the onset or progression of depression and/or a related symptom in a subject in need comprising administering to the subject a therapeutically effective amount of at least one aminosterol or a salt or derivative thereof, wherein administration of the aminosterol is via non-oral means. In one aspect, the at least one aminosterol or a salt or derivative thereof is administered via nasal, sublingual, buccal, rectal, vaginal, intravenous, intra-arterial, intradermal, intraperitoneal, intrathecal, intramuscular, epidural, intracerebral, intracerebroventricular, transdermal, or any combination thereof. In another aspect, the at least one aminosterol or a salt or derivative thereof is administered nasally.

The therapeutically effect amount of the at least one aminosterol or a salt or derivative thereof in the methods of the invention can be, for example, about 0.1 to about 20 mg/kg, about 0.1 to about 15 mg/kg, about 0.1 to about 10 mg/kg, about 0.1 to about 5 mg/kg, or about 0.1 to about 2.5 mg/kg body weight of the subject. In another aspect, the therapeutically effect amount of the at least one aminosterol or a salt or derivative thereof in the methods of the invention can be, for example, about 0.001 to about 500 mg/day, about 0.001 to about 250 mg/day, about 0.001 to about 125 mg/day, about 0.001 to about 50 mg/day, about 0.001 to about 25 mg/day, or about 0.001 to about 10 mg/day.

In another embodiment, the invention encompasses a method of treating, preventing and/or slowing the onset or progression of depression and/or a related symptom in a subject in need comprising (a) determining a dose of an aminosterol or a salt or derivative thereof for the subject, wherein the aminosterol dose is determined based on the effectiveness of the aminosterol dose in improving or resolving a depression symptom being evaluated, (b) followed by administering the aminosterol dose to the subject for a period of time, wherein the method comprises (i) identifying a depression symptom to be evaluated; (ii) identifying a starting aminosterol dose for the subject; and (iii) administering an escalating dose of the aminosterol to the subject over a period of time until an effective dose for the depression symptom being evaluated is identified, wherein the effective dose is the aminosterol dose where improvement or resolution of the depression symptom is observed, and fixing the aminosterol dose at that level for that particular depression symptom in that particular subject.

In the methods of the invention requiring aminosterol dose optimization, the aminosterol or a salt or derivative thereof can be administered via any pharmaceutically acceptable means. For example, the aminosterol or a salt or derivative thereof can be administered orally, intranasally, by injection (IV, IP, or IM) or any combination thereof. Oral and intranasal administration or a combination thereof, are preferred.

In one embodiment, starting dosages of the aminosterol or a salt or derivative thereof for oral administration can range, for example, from about 1 mg up to about 175 mg/day, or any amount in-between these two values. In another embodiment, the composition is administered orally and the dosage of the aminosterol or a salt or derivative thereof is escalated in about 25 mg increments. In yet another embodiment, the composition is administered orally and the dose of the aminosterol or a salt or derivative thereof for the subject following dose escalation is fixed at a range of from about 1 mg up to about 500 mg/day, or any amount in-between these two values.

In another embodiment, the composition is administered intranasally (IN) and the starting aminosterol or a salt or derivative thereof dosage ranges from about 0.001 mg to about 3 mg/day, or any amount in-between these two values. For example, the starting aminosterol dosage for IN administration, prior to dose escalation, can be, for example, about 0.001, about 0.005, about 0.01, about 0.02, about 0.03, about 0.05, about 0.06, about 0.07, about 0.08, about 0.09, about 0.1, about 0.15, about 0.2, about 0.25, about 0.3, about 0.35, about 0.4, about 0.45, about 0.5, about 0.55, about 0.6, about 0.65, about 0.7, about 0.75, about 0.8, about 0.85, about 0.9, about 1.0, about 1.1, about 1.25, about 1.3, about 1.4, about 1.5, about 1.6, about 1.7, about 1.75, about 1.8, about 1.9, about 2.0, about 2.1, about 2.25, about 2.3, about 2.4, about 2.5, about 2.6, about 2.7, about 2.75, about 2.8, about 2.9, or about 3 mg/day.

In another embodiment, the composition is administered intranasally and the dosage of the aminosterol or a salt or derivative thereof is escalated in increments of about 0.01, about 0.05, about 0.1, about 0.2, about 0.25, about 0.3, about 0.35, about 0.4, about 0.45, about 0.5, about 0.55, about 0.6, about 0.65, about 0.7, about 0.75, about 0.8, about 0.85, about 0.9, about 0.95, about 1, about 1.1, about 1.2, about 1.3, about 1.4, about 1.5, about 1.6, about 1.7, about 1.8, about 1.9, or about 2 mg.

Finally, in yet another embodiment, the composition is administered intranasally and the dose of the aminosterol or a salt or derivative thereof for the subject following escalation is fixed at a range of from about 0.001 mg up to about 6 mg/day, or any amount in-between these two values. In yet a further embodiment, the aminosterol composition is administered intranasally and the dose of the aminosterol or a salt or derivative thereof for the subject following dose escalation is a dose which is sub therapeutic when given orally or by injection.

In one embodiment, the dosage of the aminosterol or a salt or derivative thereof is escalated every about 3 to about 5 days. In another embodiment, the dose of the aminosterol or a salt or derivative thereof is escalated about 1×/week, about 2×/week, about every other week, or about 1×/month. In yet another embodiment, the dose of the aminosterol or a salt or derivative thereof is escalated every about 1, about 2, about 3, about 4, about 5, about 6, about 7, about 8, about 9, about 10, about 11, about 12, about 13, or about 14 days.

In another embodiment, the fixed dose of the aminosterol or a salt or derivative thereof is given once per day, every other day, once per week, twice per week, three times per week, four times per week, five times per week, six times per week, every other week, or every few days. In addition, the fixed dose of the aminosterol or a salt or derivative thereof can be administered for a first defined period of time of administration, followed by a cessation of administration for a second defined period of time, followed by resuming administration upon recurrence of depression or a symptom of depression. For example, the fixed aminosterol dose can be incrementally reduced after the fixed dose of aminosterol or a salt or derivative thereof has been administered to the subject for a period of time. Alternatively, the fixed aminosterol dose is varied plus or minus a defined amount to enable a modest reduction or increase in the fixed dose. For example, the fixed aminosterol dose can be increased or decreased by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20%.

In another embodiment, the starting aminosterol or a salt or derivative thereof dose is higher if the depression symptom being evaluated is severe.

In one aspect, where severity of the depression is reduced over a defined period of time, the reduction in severity can be measured from one or more medically-recognized techniques selected from the group consisting of the Patient Health Questionnaire-9 (PHQ-9); the Beck Depression Inventory (BDI); Zung Self-Rating Depression Scale; Center for Epidemiologic Studies-Depression Scale (CES-D); and the Hamilton Rating Scale for Depression (HRSD). The defined period of time during which the severity of the depression is reduced can be about 1 day to about 10 days, about 10 days to about 30 days, about 30 days to about 3 months, about 3 months to about 6 months, about 6 months to about 12 months, or about greater than 12 months.

In one embodiment, the method results in slowing, halting, or reversing progression or onset of depression over a defined period of time following administration of the fixed escalated dose of the aminosterol or a salt or derivative thereof, as measured by a medically-recognized technique. For example, the progression or onset of depression may be slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique. In addition, the method of the invention can result in positively impacting the depression, as measured by a medically-recognized technique. The positive impact on and/or progression of depression can be measured quantitatively or qualitatively by one or more techniques selected from the group consisting of electroencephalogram (EEG), neuroimaging, functional MRI, structural MRI, diffusion tensor imaging (DTI), [18F]fluorodeoxyglucose (FDG) PET, agents that label amyloid, [18F]F-dopa PET, radiotracer imaging, volumetric analysis of regional tissue loss, specific imaging markers of abnormal protein deposition, multimodal imaging, and biomarker analysis. In addition, the progression or onset of depression can be slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique.

In one embodiment, the fixed escalated aminosterol dose reverses dysfunction caused by the depression and treats, prevents, improves, and/or resolves the depression symptom being evaluated. In one aspect, the improvement or resolution of the depression symptom is measured using a clinically recognized scale or tool. For example, the improvement in the depression symptom can be at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, as measured using a clinically recognized scale.

In yet another embodiment, the depression symptom to be evaluated can be selected from the group consisting of (a) a symptom from the Hamilton Depression Rating Scale (HAM-D) selected from the group consisting of depressed mood, feelings of guilt, suicide, initial insomnia, middle of night insomnia, delayed insomnia, work and interests, retardation, agitation, psychic anxiety, somatic anxiety, gastrointestinal symptoms, general somatic symptoms, genital symptoms, hypochondriasis, weight loss, insight, diurnal variation, depersonalization and derealization, paranoid symptoms, and obsessional symptoms; (b) a symptom from the Montgomery-Asberg Depression Scale (MADRS) selected from the group consisting of apparent sadness, reported sadness, inner tension, reduced sleep, concentration difficulties, lassitude, inability to feel, pessimistic thoughts, and suicidal thoughts; (c) a symptom from Beck's Depression Inventory (BDI) selected from the group consisting of sadness, outlook on the future, feelings of failure, satisfaction, guilt, feelings of being punished, disappointment with self, self-blame, suicidal ideation, crying frequency, prevalence of irritation, interest in others, ease of decision-making, self-image, ability to work, ease of sleep, tiredness, appetite, weight loss, preoccupation with health, and lack of libido; (d) apathy; (e) hopelessness; (f) loss of interest in hobbies; (g) sleep problem, sleep disorder, or sleep disturbance; (h) excessive hunger; (i) lack of appetite; (j) restlessness; (k) social isolation; (l) cognitive impairment; (m) weight loss; (n) weight gain; and (o) constipation.

In one aspect, the sleep problem, sleep disorder, or sleep disturbance comprises a delay in sleep onset, sleep fragmentation, REM-behavior disorder, sleep-disordered breathing including snoring and apnea, day-time sleepiness, micro-sleep episodes, narcolepsy, early awakening, insomnia, hallucinations, or any combination thereof; In another aspect, the REM-behavior disorder comprises vivid dreams, nightmares, and acting out the dreams by speaking or screaming, or fidgeting or thrashing of arms or legs during sleep. Finally, the hallucination can comprise a visual, auditory, tactile, gustatory or olfactory hallucination.

In one embodiment, where the depression symptom to be evaluated comprises a sleep problem, sleep disorder, sleep disturbance, circadian rhythm dysfunction, REM disturbed sleep, or REM behavior disorder, then (a) the method results in a positive change in the sleeping pattern of the subject; (b) the method results in a positive change in the sleeping pattern of the subject, wherein the positive change is defined as (i) an increase in the total amount of sleep obtained of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or (ii) a percent decrease in the number of awakenings during the night selected from the group consisting of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%; and/or (c) as a result of the method the subject obtains the total number of hours of sleep recommended by a medical authority for the age group of the subject.

In one embodiment, the depression symptom to be evaluated comprises suicidal thoughts and (a) the method results in a decreased number or severity of suicidal thoughts of the subject; (b) the method results in a decreased number or severity of suicidal thoughts of the subject and the decrease in number or severity in suicidal thoughts is defined as a reduction in occurrences or severity of suicidal thoughts selected from the group consisting of by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or (c) the method results in the subject being free of suicidal thoughts.

In another embodiment, the depression symptom to be evaluated is sadness and (a) the method results in improvement in the subject's sadness, as measured by one or more clinically-recognized depression rating scale; and/or (b) the method results in improvement in the subject's sadness, as measured by one or more clinically-recognized depression rating scale, and the improvement a subject experiences following treatment is about 5, about 10, about 15, about 20, about 25, about 30, about 35, about 40, about 45, about 50, about 55, about 60, about 65, about 70, about 75, about 80, about 85, about 90, about 95 or about 100%.

In one embodiment, the depression symptom to be evaluated comprises cognitive impairment, and (a) progression or onset of the cognitive impairment is slowed, halted, or reversed over a defined period of time following administration of the fixed escalated dose of the aminosterol or a salt or derivative thereof, as measured by a medically-recognized technique; and/or (b) the cognitive impairment is positively impacted by the fixed escalated dose of the aminosterol or a salt or derivative thereof, as measured by a medically-recognized technique; (c) the cognitive impairment is positively impacted by the fixed escalated dose of the aminosterol or a salt or derivative thereof, as measured by a medically-recognized technique and the positive impact on and/or progression of cognitive impairment is measured quantitatively or qualitatively by one or more techniques selected from the group consisting of ADASCog, Mini-Mental State Exam (MMSE), Mini-cog test, Woodcock-Johnson Tests of Cognitive Abilities, Leiter International Performance Scale, Miller Analogies Test, Raven's Progressive Matrices, Wonderlic Personnel Test, IQ tests, and a computerized tested selected from Cantab Mobile, Cognigram, Cognivue, Cognision, or Automated Neuropsychological Assessment Metrics Cognitive Performance Test (CPT); and/or (d) the progression or onset of cognitive impairment is slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique.

In some embodiments, the depression symptom to be evaluated is constipation, and (a) treating the constipation prevents and/or delays the onset and/or progression of the depression; (b) the fixed escalated aminosterol dose causes the subject to have a bowel movement; (c) the method results in an increase in the frequency of bowel movement in the subject; (d) the method results in an increase in the frequency of bowel movement in the subject and the increase in the frequency of bowel movement is defined as: (i) an increase in the number of bowel movements per week of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or (ii) a percent decrease in the amount of time between each successive bowel movement selected from the group consisting of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%; (e) as a result of the method the subject has the frequency of bowel movement recommended by a medical authority for the age group of the subject; and/or (f) the starting aminosterol dose is determined by the severity of the constipation, wherein: (i) if the average complete spontaneous bowel movement (CSBM) or spontaneous bowel movement (SBM) is one or less per week, then the starting aminosterol dose is at least about 150 mg; and (ii) if the average CSBM or SBM is greater than one per week, then the starting aminosterol dose is about 75 mg or less.

In one embodiment, the depression symptom to be evaluated comprises lack of libido, and (a) the method results in treating, preventing, and/or delaying the progression and/or onset of lack of libido in the subject; (b) progression or onset of the lack of libido is slowed, halted, or reversed over a defined period of time following administration of the fixed escalated dose of the aminosterol or a salt or derivative thereof, as measured by a medically-recognized technique; (c) the lack of libido is positively impacted by the fixed escalated dose of the aminosterol or a salt or derivative thereof, as measured by a medically-recognized technique; (d) the progression of (b) and/or the positive impact of (c) is measured quantitatively or qualitatively by one or more techniques selected from the group consisting of the Sexual Desire Inventory-2 (SDI-2), Brief Index for SF Form Women, Brief Sexual Function Questionnaire for Men, Deragotis Sexual Function Inventory (DSFI), Derogatis interview for Sexual Function, Female Sexual Arousability Index, Florida Sexual History Questionnaire (FSHQ), General Information Form (GIF), Golombok Rust Inventory of Sexual Satisfaction (GRISS), Hanson Assessment of Sexual Health, Heterosexual Behavior Assessment Females, Heterosexual Behavior Assessment Males, Heterosexual Zuckerman, Homosexual Zuckerman, Hypogonadism and Sexual Function, Index of Sexual Satisfaction (ISS), International Index of Erectile Function, Jewish General Hospital Sexual Self-Monitoring Form, Leiden Impotence Questionnaire, McCoy Female Sexuality Questionnaire, Multiaxial Problem-oriented Diagnostic System of SF, Potency and Prostatectomy, Radical Prostatectomy Questionnaire, Sabbastberg Sexual Rating Scale (revised), Scalability of Sexual Experience, Segraves Sexual Symptomatology Interview, Sexual Activity of Men presenting Prostatism and Prostatectomy, Sexual Adjustment Questionnaire (SAQ), Sexual Dysfunction (Silence Hurts), Sexual Dysfunction in HIV+Men (assoc w/neuropathy/CD4 count), Sexual Dysfunction in HIV+Men, Sexual Dysfunction in Schizophrenic Patients, Sexual Function Scale, Sexual Interaction Inventory (SII), Sexual Interaction System Scale, Sexual Interest and Satisfaction Scale, Sexual Interest Questionnaire (SIQ), Sexual Inventory (SI), Sexual Orientation Method and Anxiety (SOMA), Sexual Self-Efficacy Scale for Erectile Disorder (SSES-E), Sexual Symptom Distress Scale, Sexuality Experience Scale, The Clark Sexual History Questionnaire, Urge-incontinence Impact Questionnaire, Vaginal Changes and Sexuality in Women with Cervical CA, and Watts Sexual Function Questionnaire; and/or (e) the progression or onset of (b) is slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique.

For all of the embodiments described herein, each defined period of time is independently selected from the group consisting of about 1 day to about 10 days, about 10 days to about 30 days, about 30 days to about 3 months, about 3 months to about 6 months, about 6 months to about 12 months, and about greater than 12 months.

In another embodiment, the aminosterol or a salt or derivative thereof is administered in combination with at least one additional active agent to achieve either an additive or synergistic effect. For example, the additional active agent can be administered via a method selected from the group consisting of (a) concomitantly; (b) as an admixture; (c) separately and simultaneously or concurrently; or (d) separately and sequentially. In another embodiment, the additional active agent is a different aminosterol from that administered in primary method. In yet a further embodiment, the method of the invention comprises administering a first aminosterol which is aminosterol 1436 or a salt or derivative thereof intranasally and administering a second aminosterol which is squalamine or a salt or derivative thereof orally.

In another embodiment, the at least one additional active agent is an active agent used to treat depression or a symptom thereof. In some embodiments, the active agent is selected from the group consisting of selective serotonin reuptake inhibitors (SSRIs) such as citalopram (Celexa®, Cipramil®), escitalopram (Lexapro®, Cipralex®), paroxetine (Paxil®, Seroxat®), fluoxetine (Prozac®), fluvoxamine (Luvox®, Faverin®), sertraline (Zoloft®, Lustral®), indalpine (Upstene®), zimelidine (Normud®, Zelmid®); serotonin-norepinephrine reuptake inhibitors (SNRIs) such as desvenlafaxine (Pristiq®), duloxetine (Cymbalta®), levomilnacipran (Fetzima®), milnacipran (Ixel®, Savella®), venlafaxine (Effexor®); serotonin modulators and stimulators (SMSs) such as vilazodone (Viibryd®), vortioxetine (Trintellix®); serotonin antagonists and reuptake inhibitors such as nefazodone (Dutonin®, Nefadar®, Serzone®), trazodone (Desyrel®), etoperidone; norepinephrine reuptake inhibitors (NRIs) such as reboxetine (Edronax®), teniloxazine (Lucelan®, Metatone®), viloxazine (Vivalan®), atomoxetine (Strattera®); norepinephrine-dopamine reuptake inhibitors such as bupropion (Wellbutrin®), amineptine (Survector®, Maneon®), nomifensine (Merital®, Alival®), methylphenidate (Ritalin®, Concerta®), lisdexamfetamine (Vyvanse®); tricyclic antidepressants such asamitriptyline (Elavil®, Endep®), amitriptylinoxide (Amioxid®, Ambivalon®, Equilibrin®), clomipramine (Anafranil®), desipramine (Norpramin®, Pertofrane®), dibenzepin (Noveril®, Victoril®), dimetacrine (Istonil®), dosulepin (Prothiaden®), doxepin (Adapin®, Sinequan®), imipramine (Tofranil®), lofepramine (Lomont®, Gamanil®), melitracen (Dixeran®, Melixeran®, Trausabun®), nitroxazepine (Sintamil®), nortriptyline (Pamelor®, Aventyl®), noxiptiline (Agedal®, Elronon®, Nogedal®), opipramol (Insidon®), pipofezine (Azafen®/Azaphen®), protriptyline (Vivactil®), trimipramine (Surmontil®), butriptyline (Evadyne®), demexiptiline (Deparon®, Tinoran®), fluacizine (Phtorazisin®), imipraminoxide (Imiprex®, Elepsin®), iprindole (Prondol®, Galatur®, Tertran®), metapramine (Timaxel®), propizepine (Depressin®, Vagran®), quinupramine (Kinupril®, Kevopril®), tiazesim (Altinil®), tofenacin (Elamol®, Tofacine®), amineptine (Survector®, Maneon®), tianeptine (Stablon®, Coaxil®); tetracyclic antidepressants such as amoxapine (Asendin®), maprotiline (Ludiomil®), mianserin (Bolvidon®, Norval®, Tolvon®), mirtazapine (Remeron®), setiptiline (Tecipul®), mianserin, mirtazapine, setiptiline; monoamine oxidase inhibitors (MAOIs) such as isocarboxazid (Marplan®), phenelzine (Nardil®), tranylcypromine (Parnate®), benmoxin (Neuralex®), iproclozide (Sursum®), iproniazid (Marsilid®), mebanazine (Actomol®), nialamide (Niamid®), octamoxin (Ximaol®), pheniprazine (Catron®), phenoxypropazine (Drazine®), pivhydrazine (Tersavid®), safrazine (Safra®), selegiline (Eldepryl®, Zelapar®, Emsam®), caroxazone (Surodil®, Timostenil®), metralindole (Inkazan®), moclobemide (Aurorix®, Manerix®), pirlindole (Pirazidol®), toloxatone (Humoryl®), eprobemide (Befol®), minaprine (Brantur®, Cantor®), bifemelane (Alnert®, Celeport®); atypical antipsychotics such as amisulpride (Solian®), lurasidone (Latuda®), quetiapine (Seroquel®); or N-methyl D-aspartate (NMDA) antagonists such ketamine (Ketalar®).

For all of the methods of the invention, in one embodiment each aminosterol dose is taken on an empty stomach, optionally within about two hours of the subject waking. In another embodiment for all of the methods of the invention, no food is taken or consumed after about 60 to about 90 minutes of taking the aminosterol dose. Further, in yet another embodiment applicable to all of the methods of the invention, the aminosterol or a salt or derivative thereof can be a pharmaceutically acceptable grade of at least one aminosterol or a pharmaceutically acceptable salt or derivative thereof. For all of the methods of the invention the subject can be a human.

In another embodiment, the subject to be treated according to the methods of the invention can be a member of a patient population at risk for being diagnosed with depression.

The aminosterol or a salt or derivative thereof utilized in the methods of the invention can be, for example, (a) isolated from the liver of Squalus acanthias; (b) a synthetic aminosterol; (c) squalamine or a pharmaceutically acceptable salt thereof; (d) a squalamine isomer; (e) the phosphate salt of squalamine; (f) aminosterol 1436 or a pharmaceutically acceptable salt thereof; (g) an aminosterol 1436 isomer; (h) the phosphate salt of aminosterol 1436; (i) a compound comprising a sterol nucleus and a polyamine attached at any position on the sterol, such that the molecule exhibits a net charge of at least +1; (j) a compound comprising a bile acid nucleus and a polyamine, attached at any position on the bile acid, such that the molecule exhibits a net charge of at least +1; (k) a derivative modified to include one or more of the following: (i) substitutions of the sulfate by a sulfonate, phosphate, carboxylate, or other anionic moiety chosen to circumvent metabolic removal of the sulfate moiety and oxidation of the cholesterol side chain; (ii) replacement of a hydroxyl group by a non-metabolizable polar substituent, such as a fluorine atom, to prevent its metabolic oxidation or conjugation; and (iii) substitution of one or more ring hydrogen atoms to prevent oxidative or reductive metabolism of the steroid ring system; and/or (l) a derivative of squalamine or aminosterol 1436 modified through medicinal chemistry to improve bio-distribution, ease of administration, metabolic stability, or any combination thereof. In one embodiment, the aminosterol is selected from the group consisting aminosterol 1436 or a pharmaceutically acceptable salt thereof, squalamine or a pharmaceutically acceptable salt thereof, or a combination thereof. In another embodiment, the aminosterol is a phosphate salt.

In another embodiment, the aminosterol in the methods of the invention is selected from the group consisting of:

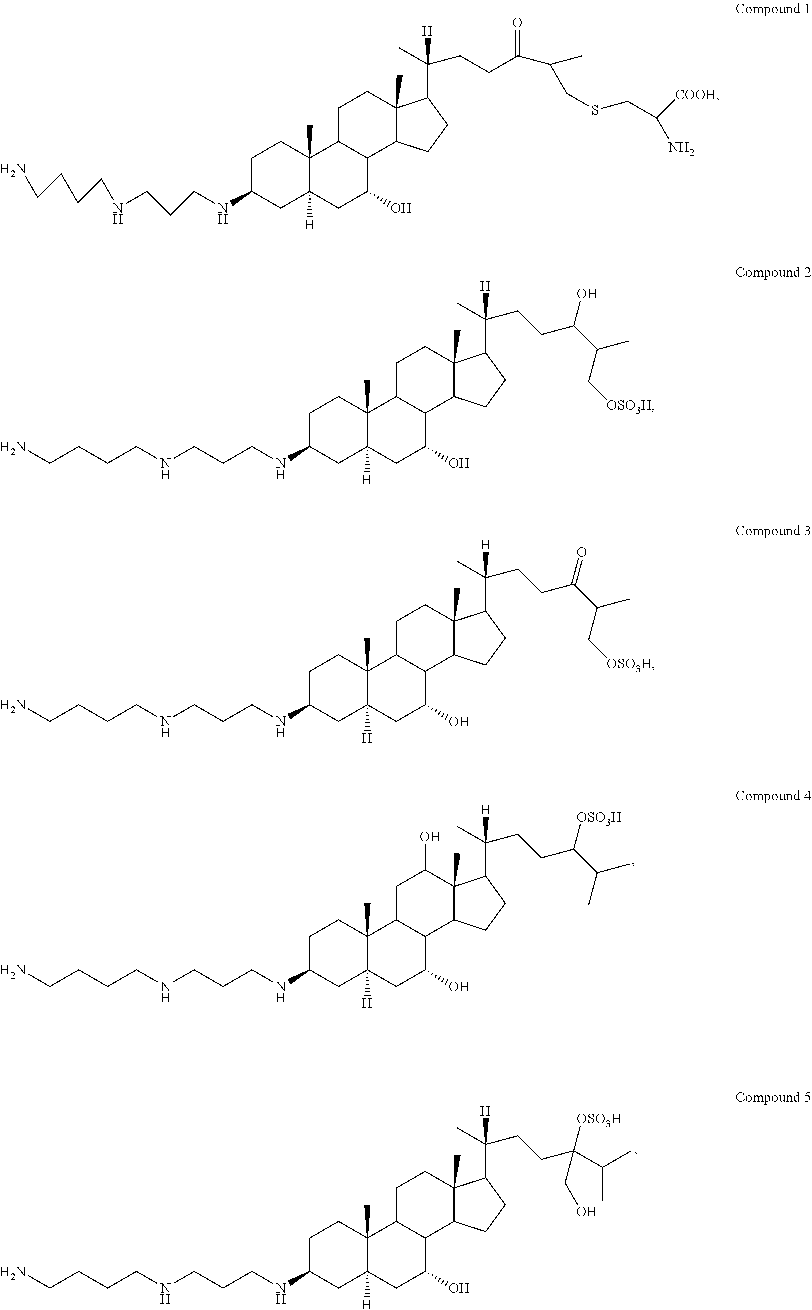

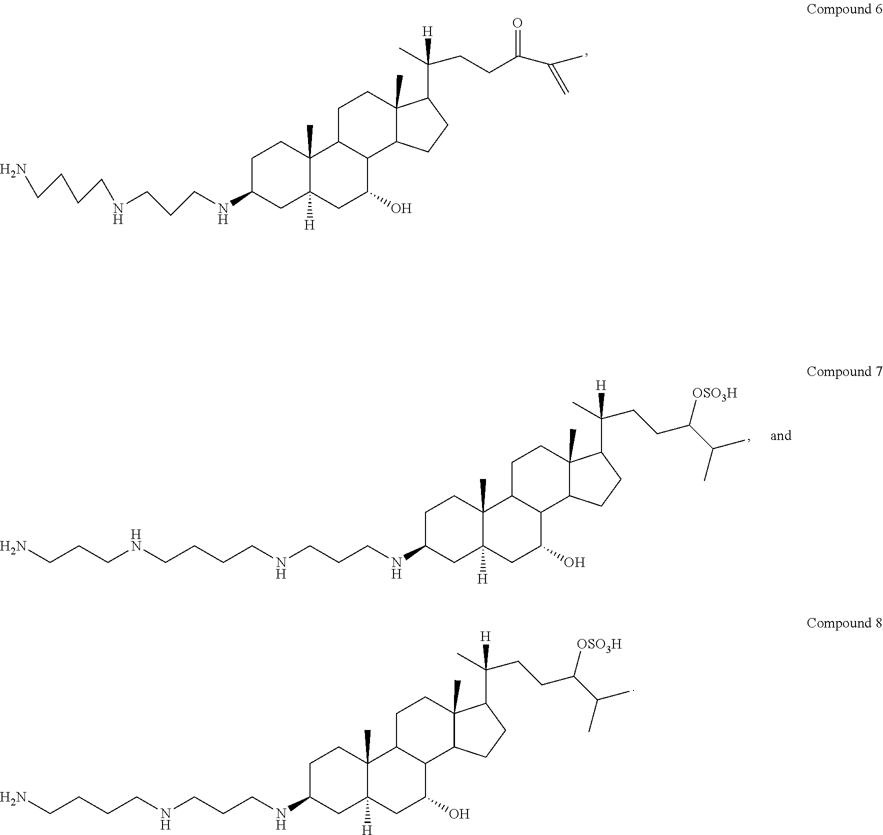

Further, the aminosterol composition can comprise, for example, one or more of the following: an aqueous carrier, a buffer, a sugar, and/or a polyol compound.

Both the foregoing summary and the following description of the drawings and detailed description are exemplary and explanatory. They are intended to provide further details of the invention, but are not to be construed as limiting. Other objects, advantages, and novel features will be readily apparent to those skilled in the art from the following detailed description of the invention.

### BRIEF DESCRIPTION OF THE DRAWINGS

FIGS. 1A and 1B show prokinetic activity of squalamine (ENT-01, a synthetic squalamine salt comprising squalamine as the active ion). As shown in panel A, in Stage 1 (single dose), cumulative prokinetic response rate was defined as the proportion of patients who had a complete spontaneous bowel movements (CSBM) within 24 hours of dosing. In Stage 2 (daily dosing), a prokinetic response was defined as the fraction of patients who had a CSBM within 24 hours of dosing on at least 2 out of 3 days at any given dose. As shown in panel B, the prokinetic dose of squalamine was significantly related to baseline constipation severity (p=0.00055). Patients with baseline CSBM<1 required a higher dose (mean, 192 mg) of squalamine than patients with CSBM≥1 (mean, 120 mg).

FIG. 2 is a schematic (flowchart) showing patient disposition in Stage 2. (1) Patients first enrolled (n=40); (2) 6 patients failed to meet dosing criteria and were excluded; (3) 34 patients were dosed; (4) 5 patients were discontinued; 3 patients withdrew consent (with 1 patient lost to follow up and 2 patients withdrew because of diarrhea); and 2 patients discontinued because of an adverse event (recurrent dizziness after medication); (5) 31 patients had an assessable prokinetic response; and (6) 29 patients completed dosing.

FIG. 3 is a chart of total sleep time in relation to squalamine dose. Total sleep time was obtained from the sleep diary by subtracting awake time during the night from total time spent in bed. Total sleep time per night was logged for each patient at baseline, each dosing period and at washout, and the means were determined. The light grey bar represents the baseline value for each cohort at a given dose level and the dark grey bar represents the value for the same cohort at the stated dose of squalamine (ENT-01; Kenterin™). The number of patients represented at each value are: Baseline, 33; 75 mg, 21; 100 mg, 28; 125 mg, 18; 150 mg, 15; 175 mg, 12; 200 mg, 7; 225 mg, 3; 250 mg, 2; washout, 33. P values were as follows: 75 mg, p=0.4; 100 mg, p=0.1; 125 mg, p=0.3; 150 mg, p=0.07; 175 mg, p=0.03; 200 mg, p=0.3; 225 mg, p=0.5; 250 mg, p=0.3; wash-out, p=0.04 (paired t test).

FIG. 4 shows the effect of squalamine (ENT-01) on circadian rhythm. The figure depicts the mean waveform of temperature under three conditions per patient: baseline (Line #1), treatment with highest drug dose (Line #2), and washout (Line #3). Each mean waveform is double plotted for better visualization. Low temperatures indicate higher activation, while higher values are associated with drowsiness and sleepiness. The top black bar indicates a standard rest period from 23:00 to 07:00 h.

FIGS. 5A-F show the effect of squalamine (ENT-01) on circadian rhythm. The figures depict the results of circadian non-parametric analysis of wrist skin temperature rhythm throughout each condition (baseline, treatment with highest dose of squalamine (ENT-01) and washout). The following parameters were measured: Inter-daily variability (FIG. 5A), inter-daily stability (IS) (FIG. 5B), relative amplitude (RA) (FIG. 5C), circadian function index (FIG. 5D), M5V (FIG. 5E), which refers to the five consecutive hours with the highest temperature or high somnolence, and L10V (FIG. 5F), which indicates the mean of the ten consecutive hours with lowest temperature or high activation. The circadian function index (CFI) is an integrated score that ranges from 0 (absence of circadian rhythm) to 1 (robust circadian rhythm). Student's paired t-test, *p<0.05, **p<01, ***p<0.001. Values expressed as mean±SEM (n=12 in each condition).

### DETAILED DESCRIPTION

### I. Overview

The present invention is directed to methods of treating, preventing and/or delaying the onset or progression of depression and/or related symptoms. In particular, the invention is directed to methods of treating, preventing and/or delaying the onset or progression of depression correlated with abnormal α-synuclein (αS) pathology. The methods comprise administering one or more aminosterols or pharmaceutically acceptable salts or derivatives thereof to a subject in need.

The aminosterol or a salt or derivative thereof can be formulated with one or more pharmaceutically acceptable carriers or excipients. Preferably the aminosterol is a pharmaceutically acceptable grade of the aminosterol.

In one embodiment, the invention encompasses a method of treating, preventing and/or slowing the onset or progression of depression and/or a related symptom in a subject in need comprising administering to the subject a therapeutically effective amount of at least one aminosterol or a salt or derivative thereof, wherein the aminosterol is administered via non-oral means. In one aspect, the at least one aminosterol or a salt or derivative thereof is administered via nasal, sublingual, buccal, rectal, vaginal, intravenous, intra-arterial, intradermal, intraperitoneal, intrathecal, intramuscular, epidural, intracerebral, intracerebroventricular, transdermal, or any combination thereof. In another aspect, the at least one aminosterol or a salt or derivative thereof is administered nasally.

In another embodiment, the present invention is directed to methods of treating, preventing and/or slowing the onset or progression of depression correlated with abnormal αS pathology or dysfunctional DA neurotransmission/dopaminergic dysfunction, comprising: (a) determining a dose of an aminosterol or a salt or derivative thereof for the subject, wherein the aminosterol dose is determined based on the effectiveness of the aminosterol dose in improving or resolving a depression symptom being evaluated; (b) followed by administering the dose of the aminosterol or a salt or derivative thereof to the subject for a period of time. The method of determining the aminosterol dose comprises (i) identifying a depression symptom to be evaluated; (ii) identifying a starting aminosterol dose for the subject; and (iii) administering an escalating dose of the aminosterol to the subject over a period of time until an effective dose for the depression symptom being evaluated is identified, wherein the effective dose is the aminosterol dose where improvement or resolution of the depression symptom is observed, and fixing the aminosterol dose at that level for that particular depression symptom in that particular subject.

It is known that αS is an important presynaptic protein regulating critical aspects of dopamine (DA) neurotransmission. Thus, the present invention is also directed to methods of treating, preventing and/or delaying the onset or progression of depression correlated with conditions related to dysfunctional DA neurotransmission, also known as dopaminergic dysfunction.

Examples of conditions or disorders correlated with depression, and which are also correlated with abnormal αS pathology, and/or dopaminergic dysfunction, include but are not limited to: (1) neurodegenerative diseases associated with neural cell death, (2) psychological or behavior disorders, and (3) cerebral and general ischemic disorders, as described in more detail below.

Depression is defined in the Psychiatric Glossary of the American Psychiatric Association as “a negative affective state, ranging from unhappiness and discontent to an extreme feeling of sadness, pessimism, and despondency, that interferes with daily life. Various physical, cognitive, and social changes also tend to co-occur, including altered eating or sleeping habits, lack of energy or motivation, difficulty concentrating or making decisions, and withdrawal from social activities. It is symptomatic of a number of mental health disorders.” The Merriam-Webster Dictionary defines depression as “a mood disorder marked especially by sadness, inactivity, difficulty in thinking and concentration, a significant increase or decrease in appetite and time spent sleeping, feelings of dejection and hopelessness, and sometimes suicidal tendencies.” Depressive disorders are associated with problems in multiple cognitive domains including attention (concentration), memory (learning), and decision making (judgment). As detailed herein, abnormal αS pathology, as well as dopaminergic dysfunction or dysfunctional DA neurotransmission, are positively correlated with depression. Administration of one or more aminosterols treats, prevents and/or slows the onset or progression of depression associated with abnormal αS pathology and/or dopaminergic dysfunction.

A. Depression

The cause of major depressive disorder is unknown. The biopsychosocial model proposes that biological, psychological, and social factors all play a role in causing depression. The diathesis-stress model specifies that depression results when a preexisting vulnerability, or diathesis, is activated by stressful life events. The preexisting vulnerability can be either genetic, implying an interaction between nature and nurture, or schematic, resulting from views of the world learned in childhood.

Depression occurs with a variety of other neurological and psychiatric disorders. The association between depression and other disorders is in part due to biological factors responsible for depression and the other disorders. For example, depression is found in 30-40% of all patients with Parkinson's disease (PD) (Frisina et al., 2009) and 40% of patients with frontotemporal dementia (FTD) (Levy et al., 1996). Before diagnosis, patients with amyotrophic lateral sclerosis (ALS) were at higher risk of receiving a clinical diagnosis of depression compared to controls (odds ratio [OR] of 1.7 and the highest risk increase was noted during the year before diagnosis with ALS) (Roos et al., 2016). Huntington's disease (HD) and depression are commonly associated conditions. In patients given a motor diagnosis of HD, 33-69% also present a depressed mood (Epping et al., 2011). The incidence of depression in schizophrenics is 61% (Gozdzik-Zelany et al., 2011). 54% of patients with multiple sclerosis (MS) met the diagnostic criteria for major depression, whereas only 14% met the diagnostic criteria that had not been diagnosed with MS (Siegert et al., 2005). Depression was also rated as present in a majority of progressive supranuclear palsy (PSP) patients (58%) (Gerstenecker et al., 2013).

The 5-HTTLPR, or serotonin transporter promoter gene's short allele has been associated with increased risk of depression. However, since the 1990s, results have been inconsistent, with three recent reviews finding an effect and two finding none. Other genes that have been linked to a gene-environment interaction include CRHR1, FKBP5 and BDNF, the first two of which are related to the stress reaction of the HPA axis, and the latter of which is involved in neurogenesis.

Depression may also come secondary to a chronic or terminal medical condition, such as HIV/AIDS or asthma, and may be labeled “secondary depression.” It is unknown whether the underlying diseases induce depression through effect on quality of life, of through shared etiologies (such as degeneration of the basal ganglia in Parkinson's disease or immune dysregulation in asthma). Depression may also be iatrogenic (the result of healthcare), such as drug-induced depression. Therapies associated with depression include interferons, beta-blockers, isotretinoin, contraceptives, cardiac agents, anticonvulsants, antimigraine drugs, antipsychotics, and hormonal agents such as gonadotropin-releasing hormone agonist. Drug abuse in early age is also associated with increased risk of developing depression later in life. Depression that occurs as a result of pregnancy is called postpartum depression, and is thought to be the result of hormonal changes associated with pregnancy. Seasonal affective disorder, a type of depression associated with seasonal changes in sunlight, is thought to be the result of decreased sunlight.

The pathophysiology of depression is not yet understood, but the current theories center around monoaminergic systems, the circadian rhythm, immunological dysfunction, HPA axis dysfunction and structural or functional abnormalities of emotional circuits. Current therapies include drugs such as selective serotonin reuptake inhibitors (SSRIs), serotonin-norepinephrine reuptake inhibitors (SNRIs), serotonin modulators and stimulators (SMSs), norepinephrine reuptake inhibitors (NRIs), norepinephrine-dopamine reuptake inhibitors, tricyclic antidepressants, and monoamine oxidase inhibitors (MAOIs). These therapies present a host of unwanted side effects, increased risk of suicide, interactions with other drugs, and discontinuation symptoms (withdrawal). Thus, treatment with aminosterols and modulation of processes related to α-synuclein presents a novel approach to the treatment and prevention of progression.

B. Depression and α-Synuclein Pathology

Many neurodiseases causing depression such as PD are suspected to correlate with the formation of toxic αS aggregates within the enteric nervous system (ENS) (Braak et al. 2003). As a result of the normal trafficking of αS aggregates from the ENS to the central nervous system (CNS) via afferent nerves such as the vagus (Holmqvist et al. 2014; Svensson et al. 2015), neurotoxic aggregates accumulate progressively within the brainstem and more rostral structures. Inhibiting αS aggregation in the ENS may, thus, reduce the continuing neuro disease process in both the ENS and CNS (Phillips et al. 2008), and thereby positively impact depression associated with abnormal αS pathology.

αS is a member of the synuclein family of soluble proteins (αS, β-synuclein and γ-synuclein) that are commonly present in CNS of vertebrates. αS is expressed in the neocortex, hippocampus, substantia niagra, thalamus and cerebellum, with the main location within the presynaptic terminals of neurons in both membrane-bound and cytosolic free forms. Presynaptic terminals release chemical messengers, called neurotransmitters, from compartments known as synaptic vesicles. The release of neurotransmitters relays signals between neurons and is critical for normal brain function. αS can be seen in neuroglial cells and melanocytic cells, and is highly expressed in the neuronal mitochondria of the olfactory bulb, hippocampus, striatum and thalamus.

αS aggregates to form insoluble fibrils in pathological conditions characterized by Lewy bodies, such as PD, dementia with Lewy bodies (DLB) and multiple system atrophy (MSA). These disorders are known as synucleinopathies. αS is the primary structural component of Lewy body fibrils. Occasionally, Lewy bodies contain tau protein; however, αS and tau constitute two distinctive subsets of filaments in the same inclusion bodies. αS pathology is also found in both sporadic and familial cases with Alzheimer's disease (AD). Thus, one indicator of abnormal αS pathology is the formation of αS aggregates.

At the molecular level, protein misfolding, accumulation, aggregation and subsequently the formation of amyloid deposits are common features in many neurological disorders including Alzheimer's disease (AD) and Parkinson's disease (PD). Thus neurodegenerative diseases are sometimes referred to as proteinopathies. The existence of a common mechanism suggests that neurodegenerative disorders likely share a common trigger and that the nature of the pathology is determined by the type of the aggregated protein and the localization of the cell affected.

Starting two decades ago with the discoveries of genetic links between αS and PD risk and the identification of aggregated αS as the main protein constituent of Lewy pathology, αS has emerged as the major therapeutic target in PD and related synucleinopathies. Brundin et al., 2017. The α-synuclein abnormalities typically found in PD are believed to be responsible for apparent catecholamine-deficits causing depression in PD (Frisina et al., 2008). In patients with PD, α-synuclein-related pathology develops in serotonergic and cholinergic neurons in parallel with that seen in the nigral dopamine neurons, and there are data to suggest that the development of cognitive impairments and depression correlate with the extent of damage seen in these systems (Wan et al, 2016). It has also been reported that AAV-mediated α-synuclein overexpression in dopamine neurons is useful to monitor depression in PD (Caudal et al, 2015).

Examples of conditions associated with abnormal αS pathology, and/or dopaminergic dysfunction, correlated with depression include, but are not limited to, synucleopathies, neurodiseases, psychological and/or behavior disorders, cerebral and general ischemic disorders, and/or disorders or conditions such as AD, PD, dementia with Lewy bodies (DLB), multiple system atrophy (MSA), Huntington's Disease, Multiple Sclerosis (MS), Amyotorphic Lateral Sclerosis (ALS), schizophrenia, Friedreich's ataxia, vascular dementia, spinal muscular atrophy, supranuclear palsy, fronto temperal dementia (FTD), progressive supranuclear palsy, Guadeloupian Parkinsonism, spinocerebellar ataxia, autism, stroke, traumatic brain injury, sleep disorders such as REM sleep behavior disorder (RBD), down syndrome, Gaucher's disease (GD), Krabbe's disease (KD), lysosomal conditions affecting glycosphingolipid metabolism, ADHD, agitation, anxiety, delirium, irritability, illusion and delusions, amnesia, apathy, bipolar disorder, disinhibition, aberrant motor and obsessive-compulsive behaviors, addiction, cerebral palsy, and epilepsy.

Several of these conditions are described in more detail below. 1. Neurodegenerative Diseases Associated with Neural Cell Death

The methods and compositions of the invention may also be useful in treating, preventing, and/or delaying the onset or progression of depression and/or a depression-related symptom, where the depression is correlated with abnormal α-synuclein (αS) pathology, and/or correlated with dopaminergic dysfunction, where the depression is also correlated with neurodegenerative diseases associated with cell death. Nom-limiting examples of neurodegenerative diseases associated with cell death are described below.

i. Synucleinopathies

Synucleinopathies (also called α-Synucleinopathies) are neurodegenerative diseases characterized by the abnormal accumulation of fibrillary aggregates of αS protein in the cytoplasm of selective populations of neurons and glia. These disorders include PD, dementia with Lewy-bodies (DLB), pure autonomic failure (PAF), and MSA. Other rare disorders, such as various neuroaxonal dystrophies, also have αS pathologies.

The synucleinopathies have shared features of depression, as well as parkinsonism, sleep disorders, and visual hallucinations. Synucleinopathies can sometimes overlap with tauopathies, possibly because of interaction between the synuclein and tau proteins.

αS deposits can affect the cardiac muscle and blood vessels. Almost all people with synucleinopathies have cardiovascular dysfunction, although most are asymptomatic. From chewing to defecation, αS deposits affect every level of gastrointestinal function. Symptoms include upper gastrointestinal tract dysfunction such as delayed gastric emptying or lower gastorintestinal dysfunction, such as constipation and prolonged stool transit time.

Urinary retention, waking at night to urinate, increased urinary frequency and urgency, and over- or underactive bladder are common in people with synucleinopathies. Sexual dysfunction usually appears early in synucleinopathies, and may include erectile dysfunction, and difficulties achieving orgasm or ejaculating.

Depression associated with age-related neurodegenerative diseases remains a significant unsolved problem and challenge. The number of people over 60 years is expected to rise from 841 million in 2013 to more than 2 billion in 2050 (United Nations. World population ageing 2013). As populations get older, age-related neurodegenerative diseases such as PD have become more common (Reitz et al. 2011; Reeve et al. 2014). Even for less common neurodegenerative diseases, such as ALS, this trend of increased incidence seems likely (Beghi et al. 2006).

ii. Frontotemporal Dementia (FTD)

Frontotemporal dementia (FTD) or frontotemporal degenerations is a clinical term that refers to a group of progressive neurodegenerative disorders that affect the frontal and temporal lobes causing personality change (apathy, disinhibition, loss of insight and emotional control), loss of the ability to recognize the meaning of words and objects, language dysfunction, and global cognitive decline. FTD causes atrophy in the part of the brain that controls judgment, behavior and executive function. FTDs have an age of onset of 40-50 years and, at an early stage, do not cause memory loss and visuo-spatial disorientation. There is an overlap between FTDs, amyotrophic lateral sclerosis (ALS), and atypical parkinsonian syndromes (progressive supranuclear palsy and corticobasal degeneration).

In FTD, the nerve cell loss is most prominent in areas that control conduct, judgment, empathy and foresight, among other abilities. Primary progressive aphasia (PPA) is the second major form of frontotemporal degeneration that affects language skills, speaking, writing and comprehension. PPA normally comes on in midlife, before age 65, but can occur in late life also.

Prior studies have reported the presence of tau and αS inclusions in a case of FTD and progressive aphasia. Yancopoulou et al., 2005. Similarly, a more resent study reported the significant presence of phosphorylated αS-positive structures were also found in oligodendrocytes and neuropil of FTD patients (Hosokawa et al., 2017).

iii. Amyotrophic Lateral Sclerosis (ALS)

Amyotrophic lateral sclerosis (ALS), also known as motor neuron disease (MND), or Lou Gehrig's disease, is a specific disease which causes the death of neurons controlling voluntary muscles. ALS is characterized by stiff muscles, muscle twitching, and gradually worsening weakness due to muscles decreasing in size. This results in difficulty speaking, swallowing, and eventually breathing. The cause is not known in 90% to 95% of cases. The remaining 5-10% of cases are genetic. The underlying mechanism involves damage to both upper and lower motor neurons. No cure for ALS is known. The disease can affect people of any age, but usually starts around the age of 60 and in inherited cases around the age of 50. The average survival from onset to death is 2 to 4 years, although about 10% survive longer than 10 years.

Although the degeneration predominantly affects the motor system, cognitive and behavioral symptoms have been described for over a century, and there is evidence that ALS and frontotemporal dementia overlap clinically, radiologically, pathologically, and genetically. Cognitive decline in ALS is characterized by personality change, irritability, obsessions, poor insight, and pervasive deficits in frontal executive tests. This presentation is consistent with the changes to character, social conduct, and executive function in frontotemporal dementia (Phukan et al., 2007).

αS pathology has been examined in the brains and spinal cords of patients with ALS/parkinsonism-dementia complex (PDC) (Kokubo et al. 2012). This study reported that various types of phosphorylated αS-positive structures were found in all ALS/PDC cases. This is significant as phosphorylated αS is the main component of Lewy bodies (LBs) that are characteristic of PD and DLB.

iv. Huntington's Disease (HD)

Huntington's disease (HD) is a progressive brain disorder caused by a defective gene. This disease causes changes in the central area of the brain, which affect movement, mood and thinking skills. HD is a progressive brain disorder caused by a single defective gene on chromosome 4—one of the 23 human chromosomes that carry a person's entire genetic code. This defect is “dominant,” meaning that anyone who inherits it from a parent with Huntington's will eventually develop the disease.

The hallmark symptom of HD is uncontrolled movement of the arms, legs, head, face and upper body. HD also causes a decline in thinking and reasoning skills, including memory, concentration, judgment, and ability to plan and organize.

αS also plays a role in the disease pathology of HD. Specifically, recent studies report that αS levels modulate HD in mice (Corrochano et al., December 2012). Similarly, yet another study reported that αS levels affect autophagosome numbers in vivo and modulate HD pathology (Corrochano et al., March 2012).

v. Schizophrenia

Schizophrenia is a chronic progressive disorder that has at its origin structural brain changes in both white and gray matter. The incidence of depression in schizophrenics is 61% (Gozdzik-Zelany et al., 2011). It is likely that these changes in white and gray matter begin prior to the onset of clinical symptoms in cortical regions, particularly those concerned with language processing. Later, they can be detected by progressive ventricular enlargement. Current magnetic resonance imaging (MRI) technology can provide a valuable tool for detecting early changes in cortical atrophy and anomalous language processing, which may be predictive of who will develop schizophrenia.

The duration and strength of the dopaminergic signal are regulated by the dopamine transporter (DAT). Drug addiction and neurodegenerative and neuropsychiatric diseases including schizophrenia have all been associated with altered DAT activity. αS, a protein partner of DAT, is implicated in neurodegenerative disease and drug addiction.

A recent study reported that patients with schizophrenia exhibit a decreased expression of αS. Demirel et al. 2017. Specifically, the study reported that schizophrenia subjects exhibited significantly lower serum levels of αS as compared to healthy controls. As serum αS plays a neuromodulator role, this lower amount may result in impaired neuroplasticity in the etiology of schizophrenia, as well as noticeable depression in schizophrenia which progresses over time.

vi. Multiple Sclerosis

Multiple sclerosis (MS) is a demyelinating disease in which the insulating covers of nerve cells in the brain and spinal cord are damaged. This damage disrupts the ability of parts of the nervous system to communicate, resulting in a range of signs and symptoms, including physical, mental, and sometimes psychiatric problems. Specific symptoms can include double vision, blindness in one eye, muscle weakness, trouble with sensation, or trouble with coordination. MS takes several forms, with new symptoms either occurring in isolated attacks (relapsing forms) or building up over time (progressive forms). Between attacks, symptoms may disappear completely; however, permanent neurological problems often remain, especially as the disease advances. There is no known cure for MS. Life expectancy is on average 5 to 10 years lower than that of an unaffected population. MS is the most common immune-mediated disorder affecting the central nervous system. In 2015, about 2.3 million people were affected globally, and in 2015 about 18,900 people died from MS, up from 12,000 in 1990.

As MS progresses, usually with a series of acute immune attacks and a late-stage steady march of function loss, patients with MS commonly experience fatigue, spasticity, difficulty walking, and cognitive impairment (Rahn et al., 2012).

Abnormal αS pathology is correlated with MS. Specifically, a recent study reported that levels of αS in the cerebrosinal fluid (CSF) of MS subjects was significantly lower as compared to healthy controls (Antonelou et al., 2015). Similarly, a more recent study reported the low levels of αS in peripheral tissues are related to clinical relapse in relapse-remitting MS (Mejia et al., 2018). Finally, it is believed that alpha-synuclein expression regulated by inflammatory signals may contribute to neurodegenerative processes in MS lesions (Lu et al., 2009). Overexpression of αS is known to be associated with depression as discussed herein, thus MS and depression may share mechanistic overlap.

vii. Various Other Conditions

Progressive supranuclear palsy (PSP), also called Steele-Richardson-Olszewski syndrome, is a brain disorder that causes problems with walking, balance and eye movements. The disorder results from deterioration of cells in areas of the brain that control body movement and thinking. There is no known cure for PSP and management is primarily supportive. Depression is a common condition occurring in patients with PSP and patients may display depressive symptoms either before or after manifestation of PSP (Menza et al., 1995; Kim et al., 2009; and Esmonde et al., 1996). PSP is considered a sporadic neurodegenerative disease, one that develops by chance. Build-up of the tau protein in the brain causes cellular damage and thus affects the normal function of neurons. PSP is considered a tauopathy, while depression has also been observed to correlate with increased tau levels in the brain (Gatchel et al, 2017). Build-up of the tau protein in PSP is significant. Researchers have reported that tau and αS build-up appears to promote the fibrillization and solubility of each other in vitro and in vivo. This suggests that interactions between tau and αS form a deleterious feed-forward loop essential for the development and spreading of neurodegeneration (Moussaud et al. 2014).

Vascular dementia, also known as multi-infarct dementia (MID) and vascular cognitive impairment (VCI), is dementia caused by problems in the supply of blood to the brain, typically a series of minor strokes, leading to worsening cognitive decline that occurs step by step. Depression occurs in 25-80% of VCI patients (Cummings et al., 1988). Risk factors for vascular dementia include age, hypertension, smoking, hypercholesterolemia, diabetes mellitus, cardiovascular disease, and cerebrovascular disease. Other risk factors include geographic origin, genetic predisposition, and prior strokes. Vascular dementia is not a single entity, but an umbrella term to describe cognitive decline due to a series of different vessel disorders, frequently seen in combination with other non-vascular changes. These vessel disorders can induce various types of cerebral tissue lesions such as hemorrhage, infarction, hippocampal sclerosis, and white matter lesions. It is believed that vascular pathology is a common cause of both depression and vascular cognitive impairment (Morimoto et al., 2015)

Spinal muscular atrophy (SMA) is an inherited neuromuscular disorder characterized by loss of motor neurons and progressive muscle wasting, often leading to early death. The disorder is caused by a genetic defect in the SMN1 gene, which encodes SMN, a protein necessary for survival of motor neurons. Lower levels of the protein results in loss of function of neuronal cells in the anterior horn of the spinal cord and subsequent system-wide atrophy of skeletal muscles. It has been reported that significantly lower αS expression were found in tissue samples of SMA patients, suggesting a αS related dysfunction contributes to the disease pathology (Acsadi et al., 2011).

Friedreich's ataxia (FRDA) is an autosomal recessive inherited disease that causes progressive damage to the nervous system. It manifests in initial symptoms of poor coordination such as gait disturbance; it can also lead to scoliosis, heart disease and diabetes. The ataxia of Friedreich's ataxia results from the degeneration of nervous tissue in the spinal cord, in particular sensory neurons essential (through connections with the cerebellum) for directing muscle movement of the arms and legs. The spinal cord becomes thinner and nerve cells lose some of their myelin sheath (the insulating covering on some nerve cells that helps conduct nerve impulses). Depression is common in those with FRDA, and it is believed that depression is not simply a reaction to the disease but associated with structural pathology in the brain (Silva et al., 2013).

2. Psychological or Behavior Disorders

The methods and compositions of the invention may also be useful in treating, preventing, and/or delaying the onset or progression of depression and/or a depression-related symptom, where the depression is correlated with abnormal α-synuclein (αS) pathology, and/or correlated with dopaminergic dysfunction, where the depression is also correlated with psychological or behavior disorders. Nom-limiting examples of psychological or behavior disorders are described below.

i. Sleep Disorders & Sleep Disturbances

Studies have found a correlation between sleep disorders, sleep disturbances and/or sleep fragmentation and depression. Sleep disorders and depression are frequent in patients with the syncleinopathy, PD (Happe et al., 2008). About three quarters of depressed patients have insomnia symptoms, and hypersomnia is present in about 40% of young depressed adults and 10% of older patients, with a preponderance in females (Nutt et al., 2008). Depressed patients often show altered circadian rhythms, sleep disturbances, and diurnal mood variation (Germain et al., 2008). Also, sleep and circadian rhythm alterations correlate with depression and cognitive impairment in Huntington's disease (Aziz et al., 2010). In yet another study, it was reported that αS overexpression in mice produces sleep disruptions (McDowell et al. 2014).

REM sleep behavior disorder (RBD) is a parasomnia in which individuals with RBD lose the paralysis of muscles (atonia) that is normal during rapid eye movement (REM) sleep, and act out their dreams or have other abnormal movements or vocalizations. Abnormal sleep behaviors may appear decades before any other symptoms, often as an early sign of a synucleinopathy. On autopsy, 94 to 98% of individuals with polysomnography-confirmed RBD are found to have a synucleinopathy—most commonly DLB or PD. Other symptoms of the specific synucleinopathy usually manifest within 15 years of the diagnosis of RBD, but may emerge up to 50 years after RBD diagnosis.

ii. Autism

Autism, or autism spectrum disorder (ASD), refers to a range of conditions characterized by challenges with social skills, repetitive behaviors, speech and nonverbal communication, as well as by unique strengths and differences. There are many types of autism, caused by different combinations of genetic and environmental influences. One trait characteristic of many autism subjects is depression (Ghaziuddin et al., 2002).

The Centers for Disease Control and Prevention (CDC) estimates autism's prevalence as 1 in 59 children in the United States. This includes 1 in 37 boys and 1 in 151 girls. Around one third of people with autism remain nonverbal, and around one third of people with autism have an intellectual disability. Certain medical and mental health issues frequently accompany autism. They include gastrointestinal (GI) disorders, seizures, sleep disturbances, attention deficit and hyperactivity disorder (ADHD), anxiety and phobias.

A recent brain-tissue study suggests that children affected by autism have a surplus of synapses, or connections between brain cells. The excess is due to a slowdown in the normal pruning process that occurs during brain development. During normal brain development, a burst of synapse formation occurs in infancy. This is particularly pronounced in the cortex, which is central to thought and processing information from the senses. But by late adolescence, pruning eliminates about half of these cortical synapses. In addition, many genes linked to autism are known to affect the development or function of brain synapses. The study also found that the brain cells from individuals with autism were filled with damaged parts and deficient in signs of a normal breakdown pathway called “autophagy” (Tang et al. 2014).

Abnormal αS pathology plays a role in ASD. In particular, a recent study reported that mean plasma αS levels were significantly lower autism spectrum disorder (ASD) children as compared to healthy controls while b-synuclein levels were higher in ASD subjects vs controls (Sriwimol et al., 2018). As serum as synuclein proteins play a neuromedulator role, these abnormalities may correlate with depression observed with ASD subjects.

iii. Cognitive Impairment

Cognitive impairment is frequently associated with abnormal αS pathology, and this condition can also correlate with depression. Moreover, depressive disorders are associated with problems in multiple cognitive domains including attention (concentration), memory (learning), and decision making (judgment) (Rubin et al., 2016).

Cognitive impairment (CI) is a common non-motor complication of Parkinson disease (PD), and a postmortem analysis of PD subjects found that a higher prevalence of pathological features were present in depressed compared to non-depressed PD patients (Frisina et al., 2009). This is not surprising as αS is a neuronal protein involved in the regulation of brain serotonin and dopamine levels (Frieling et al., 2008). αS overexpression in mice leads to cognitive impairment (Magen et al., 2011).

3. Ischemic Disorders

The methods and compositions of the invention may also be useful in treating, preventing, and/or delaying the onset or progression of depression and/or a depression-related symptom, where the depression is correlated with abnormal α-synuclein (αS) pathology, and/or correlated with dopaminergic dysfunction, where the depression is also correlated with a cerebral or general ischemic disorder.

In some embodiments, the cerebral ischemic disorder comprises cerebral microangiopathy, intrapartal cerebral ischemia, cerebral ischemia during/after cardiac arrest or resuscitation, cerebral ischemia due to intraoperative problems, cerebral ischemia during carotid surgery, chronic cerebral ischemia due to stenosis of blood-supplying arteries to the brain, sinus thrombosis or thrombosis of cerebral veins, cerebral vessel malformations, or diabetic retinopathy.

In some embodiments, the general ischemic disorders comprises high blood pressure, high cholesterol, myocardial infarction, cardiac insufficiency, cardiac failure, congestive heart failure, myocarditis, pericarditis, perimyocarditis, coronary heart disease, angina pectoris, congenital heart disease, shock, ischemia of extremities, stenosis of renal arteries, diabetic retinopathy, thrombosis associated with malaria, artificial heart valves, anemias, hypersplenic syndrome, emphysema, lung fibrosis, or pulmonary edema.

Depression has been correlated with ischemic disorders. Major depression is a common comorbidity associated with ischemic heart disease (IHD) (O'connor, et al., 2000). Depression effects up to 40% of patients experiencing ischemic stroke. Patients that experienced depression and strokes have displayed increased number of infarcts affecting prefrontosubcortical circuits than patients not displaying depression (Vatagja et al., 2001). Major depression is associated with an increased risk of coronary artery disease and acute cardiovascular sequelae, such as myocardial infarction, congestive heart failure, and isolated systolic hypertension (Nemeroff et al., 2012).

Studies have also shown a correlation between abnormal αS pathology and ischemic disorders. For example, one study reported that post-stroke induction of αS mediates ischemic brain damage (Kim et al. 2016). Yet another study conducted a comparison of the amount of αS in ischemic stroke and PD subjects, with the results showing that the levels of oligomeric form of αS of red blood cells in ischemic stroke and PD patients were both significantly higher than that of healthy controls (Zhao et al., 2016). Finally, another study reported that cerebral ischemic injury leads to a reduction in αS and consequently causes serious brain damage (Kho, 2017).

C. Summary of Experimental Results

As described in Example 1, a study was conducted in patients with Parkinson's disease (PD). PD is a progressive neurodegenerative disorder caused by accumulation of the protein α-synuclein (αS) within the enteric nervous system (ENS), autonomic nerves and brain.

While the study described herein assessed patients with PD, symptoms assessed and contemplated to be resolved by aminosterol treatment, such as depression, are not restored by the replacement of dopamine and are thus not unique to PD but rather common across a variety of disorders which involve impaired function of neural pathways, related to abnormal αS pathology, referred to herein as “brain-gut” disorders. Depression results from impaired function of neural pathways not restored by replacement of dopamine.

The methods and compositions disclosed herein permit exerting pharmacological control over the ENS in a manner that is without precedent in the literature. A strategy that targets neurotoxic aggregates of αS in the GI tract represents a novel approach to the treatment of depression correlated with abnormal αS pathology and/or correlated with dysfunctional DA neurotransmission/dopaminergic dysfunction. Treatment and conditions described herein may restore the function of enteric nerve cells and prevent retrograde trafficking to the brain. Such actions may potentially slow progression of depression and/or the underlying disease or condition.

Most surprisingly, as described in Example 1, it was discovered that aminosterol dosing is patient specific, as the dose is likely related to the extent of neuronal damage, with greater neuronal damage correlating with the need for a higher aminosterol dose to obtain a desired therapeutic result (e.g., treating depression). This was not known prior to the present invention. Thus, one aspect of the present invention is directed to methods of treating, preventing, and/or slowing the onset or progression of depression and/or a depression related symptom in a subject in need, where the method comprises determining an effective therapeutic aminosterol dose for the subject. The method comprises a first step of identifying a depression-related symptom to be evaluated for determining the effective therapeutic aminosterol dose for the subject.

In addition, it was also surprisingly discovered that the starting dose of the aminosterol or a salt or derivative thereof is dependent upon the severity of depression and/or a depression related symptom. Specifically, if the depression and/or a depression related symptom is severe, then the starting aminosterol dose, prior to dose escalation, should be higher than if the depression and/or a depression related symptom is moderate. “Severe” depression can be determined by a clinical scale or tool appropriate for measuring the identified depression and/or a depression related symptom.

One impact of the present invention is that recognizing that an aminosterol dose useful in treating depression and/or a depression related symptom is patient specific can prevent the use of incorrect aminosterol doses for patients. This is a significant discovery, as if a subject is put on an aminosterol dose that is too high, then resultant nausea, vomiting, and abdominal discomfort can result in the patient going off the drug, with the depression and/or a depression related symptoms remaining untreated. Similarly, if a subject is put on an aminosterol dose that is too low, then the depression and/or a depression related symptoms will not be successfully treated. Prior to the present invention, there was no recognition that therapeutically effective aminosterol doses had no relation to the sex, age, weight, ethnicity, or other similar patient characteristics. This is unexpected, as it is contrary to dosing strategies for almost all other medications.

Not to be bound by theory, it is believed that aminosterols target neurotoxic aggregates of αS in the gastrointestinal tract, and restore function of the enteric nerve cells. The now-functional enteric nerve cells prevent retrograde trafficking of proteins, such as alpha-synuclein, to the brain. In addition to restoring gastrointestinal function, this effect is believed to slow and possibly reverse depression progression.

Not to be bound by theory, based on the data described herein, it is believed that aminosterols improve bowel function by acting locally on the gastrointestinal tract (as supported by the oral bioavailability <0.3%). An orally administered aminosterol such as squalamine, the active ion of ENT-01, stimulates gastro-intestinal motility in mice with constipation due to overexpression of human αS (West et al, manuscript in preparation). Perfusion of an aminosterol such as squalamine through the lumen of an isolated segment of bowel from the PD mouse model results in excitation of IPANs (intrinsic primary afferent neuron), the major sensory neurons of the ENS that communicate with the myenteric plexus, increasing the frequency of propulsive peristaltic contractions and augmenting neural signals projecting to the afferent arm of the vagus.

Systemic absorption of the aminosterol following oral administration was negligible both in this study and in prior studies involving mice, rats and dogs. Prior studies demonstrated that intravenous administration of squalamine was not associated with increased gastrointestinal motility, despite reaching systemic blood levels one thousand-fold greater than that achieved by orally administered squalamine. These data suggest that the effect is mediated by local action in the GI tract. The topical action would also explain why adverse events were largely confined to the gastrointestinal tract.

Several exploratory endpoints were incorporated into the trial described in Example 1 to evaluate the impact of an aminosterol on neurologic symptoms associated with a neurological disease such as PD. Following aminosterol treatment, the Unified Parkinson's Disease Rating Scale (UPDRS) score, a global assessment of motor and non-motor symptoms, showed significant improvement. Improvement was also seen in the motor component. The improvement in the motor component is unlikely to be due to improved gastric motility and increased absorption of dopaminergic medications, since improvement persisted during the 2-week wash-out period, i.e., in the absence of study drug (Table 12).

Improvements were also seen in cognitive function (MMSE scores), hallucinations, REM-behavior disorder (RBD) and sleep. Six of the patients enrolled had daily hallucinations or delusions and these improved or disappeared during treatment in five. In one patient the hallucinations disappeared at 100 mg, despite not having reached the colonic prokinetic dose (e.g., fixed escalated aminosterol dose) of 175 mg for this particular patient. The patient remained free of hallucinations for 1 month following cessation of dosing. RBD and total sleep time also improved progressively in a dose-dependent manner.

Interestingly, most indices related to bowel function returned to baseline value by the end of the 2-week wash-out period while improvement in the CNS symptoms persisted. The rapid improvement in certain CNS symptoms is consistent with a mechanism whereby nerve impulses initiated from the ENS following aminosterol administration augment afferent neural signaling to the CNS. This may stimulate the clearance of αS aggregates within the afferent neurons themselves as well as the secondary and tertiary neurons projecting rostrally within the CNS, since it is known that neural stimulation is accompanied by increased neuronal autophagic activity (Shehata et al. 2012). It is believed that after cessation of aminosterol administration, the neurons of the CNS gradually re-accumulate an αS burden either locally or via trafficking from αS re-aggregation within the gut.

Low Bioavailability:

As described in Example 1, in preclinical studies, squalamine (ENT-01) exhibited an oral bioavailability of about 0.1% in both rats and dogs. In Stage 1 of the phase 2 study, oral dosing up to 200 mg (114 mg/m²) yielded an approximate oral bioavailability of about 0.1%, based on a comparison of a pharmacokinetic data of the oral dosing and the pharmacokinetic data measured during prior phase 1 studies of IV administration of squalamine. Thus, in one embodiment of the invention, aminosterol dosing, either oral or intranasal, results in a bioavailability of less than about 3%, less than about 2.5%, less than about 2%, less than about 1.5%, less than about 1%, less than about 0.9%, less than about 0.8%, less than about 0.7%, less than about 0.6%, less than about 0.5%, less than about 0.4%, less than about 0.3%, less than about 0.2%, or about 0.1% or less.

As described in Example 1, aminosterol dosing is patient specific, as the dose is likely related to the extent of neuronal damage, with greater neuronal damage correlating with the need for a higher aminosterol dose to obtain a desired therapeutic result. As described in greater detail herein, aminosterol dosing can range from about 0.01 to about 500 mg/day, with dosage determination described in more detail below.

### II. Methods of Treatment

The present application provides methods for the treatment of depression using aminosterols. Thus, in one aspect a method of treating, preventing, and/or slowing progression of depression and/or a related symptom in a subject in need is provided, the method comprising administering to the subject a therapeutically effective amount of at least one aminosterol, or a salt or derivative thereof, provided that the administering does not comprise oral administration.

Administration may be via any route of administration other than oral administration. Non-limiting examples include nasal, sublingual, buccal, rectal, vaginal, intravenous, intra-arterial, intradermal, intraperitoneal, intrathecal, intramuscular, epidural, intracerebral, intracerebroventricular, transdermal, or any combination thereof.

In one embodiment, administering comprises nasal administration. Nasal administration may be accomplished via insufflation of solids or powders, or via inhalation of a mist comprising the at least one aminosterol, or a salt or derivative thereof, in a suitable carrier and optionally excipients. Suitable carriers and excipients are known to the skilled artisan and include buffers such as sodium phosphate, sodium citrate, and citric acid; solubilizers such as glycols, small quantities of alcohol, transcutol (diethylene glycol monoethyl ether), medium chain glycerides, labrasol (saturated polyglycolyzed C₈-C₁₀ glyceride), surfactants and cyclodextrins; preservatives such as parabens, phenyl ethyl alcohol, benzalkonium chloride, EDTA (ethylene diaminetetraaceticacid), and benzoyl alcohol; antioxidants such as sodium bisulfite, butylated hydroxytoluene, sodium metabisulfite and tocopherol; humectants such as glycerin, sorbitol and mannitol; surfactants such as polysorbet; bioadhesive polymers such as mucoadhesives; and penetration enhancers such as dimethyl sulfoxide (DMSO).

Nasal administration via inhalation of a mist may employ the use of metered-dose spray pumps. Typical volumes of aminosterol comprising mist, delivered via a single pump of a metered-dose spray pump may be about 20-100 μl, 100-150 μl, or 150-200 μl. Such pumps offer high reproducibility of the emitted dose and plume geometry in in vitro tests. The particle size and plume geometry can vary within certain limits and depend on the properties of the pump, the formulation, the orifice of the actuator, and the force applied.

In some embodiments, the therapeutically effective amount of the at least one aminosterol, or a salt or derivative thereof comprises about 0.1 to about 20 mg/kg body weight of the subject. In some embodiments, the therapeutically effective amount of the at least one aminosterol, or a salt or derivative thereof comprises about 0.1 to about 5 mg/kg body weight of the subject. In some embodiments, the therapeutically effective amount of the at least one aminosterol, or a salt or derivative thereof comprises about 5 to about 10 mg/kg body weight of the subject. In some embodiments, the therapeutically effective amount of the at least one aminosterol, or a salt or derivative thereof comprises about 10 to about 15 mg/kg body weight of the subject. In some embodiments, the therapeutically effective amount of the at least one aminosterol, or a salt or derivative thereof comprises about 15 to about 20 mg/kg body weight of the subject.

In some embodiments, the therapeutically effective amount of the at least one aminosterol, or a salt or derivative thereof comprises about 0.1 to about 20 mg/kg body weight of the subject. In some embodiments, the therapeutically effective amount of the at least one aminosterol, or a salt or derivative thereof comprises about 0.1 to about 15 mg/kg body weight of the subject. In some embodiments, the therapeutically effective amount of the at least one aminosterol, or a salt or derivative thereof comprises about 0.1 to about 10 mg/kg body weight of the subject. In some embodiments, the therapeutically effective amount of the at least one aminosterol, or a salt or derivative thereof comprises about 0.1 to about 5 mg/kg body weight of the subject.

In some embodiments, the therapeutically effective amount of the at least one aminosterol, or a salt or derivative thereof comprises about 0.001 to about 2 mg/day. In some embodiments, the therapeutically effective amount of the at least one aminosterol, or a salt or derivative thereof comprises about 2 to about 4 mg/day. In some embodiments, the therapeutically effective amount of the at least one aminosterol, or a salt or derivative thereof comprises about 4 to about 6 mg/day. In some embodiments, the therapeutically effective amount of the at least one aminosterol, or a salt or derivative thereof comprises about 0.001 to about 6 mg/day.

In some embodiments, the therapeutically effective amount of the at least one aminosterol, or a salt or derivative thereof comprises about 0.001 to about 4 mg/day. In some embodiments, the therapeutically effective amount of the at least one aminosterol, or a salt or derivative thereof comprises about 0.001 to about 2 mg/day. In some embodiments, the therapeutically effective amount of the at least one aminosterol, or a salt or derivative thereof comprises about 0.001 to about 1 mg/day.

In another embodiments, the therapeutically effective amount of the at least one aminosterol, or a salt or derivative thereof comprises about 0.001, about 0.005, about 0.01, about 0.02, about 0.03, about 0.04, about 0.05, about 0.06, about 0.07, about 0.08, about 0.09, about 0.1, about 0.2, about 0.3, about 0.4, about 0.5, about 0.6, about 0.7, about 0.8, about 0.9, about 1, about 1.1, about 1.2, about 1.3, about 1.4, about 1.5, about 1.6, about 1.7, about 1.8, about 1.9, about 2, about 2.1, about 2.2, about 2.3, about 2.4, about 2.5, about 2.6, about 2.7, about 2.8, about 2.9, about 3, about 3.1, about 3.2, about 3.3, about 3.4, about 3.5, about 3.6, about 3.7, about 3.8, about 3.9, about 4, about 4.1, about 4.2, about 4.3, about 4.4, about 4.5, about 4.6, about 4.7, about 4.8, about 4.9, about 5, about 5.1, about 5.2, about 5.3, about 5.4, about 5.5, about 5.6, about 5.7, about 5.8, about 5.9, or about 6 mg/day.

### III. Methods of Determining a “Fixed Dose” of an Aminosterol

The present application relates to the surprising discovery of a method to determine a “fixed dose” of an aminosterol composition useful in treating, preventing, and/or delaying onset depression or a relayed symptom, where the dose is not age, size, or weight dependent but rather is individually calibrated. In one embodiment, the depression is correlated with abnormal αS pathology or dysfunctional DA neurotransmission/dopaminergic dysfunction. The “fixed aminosterol dose” obtained through this method yields highly effective results in treating the symptom(s) based on which the “fixed aminosterol dose” was determined, related symptoms along the “brain-gut” axis, and the underlying depression. Further, contemplated herein are methods of leveraging this same “fixed dose” method for methods of prevention of depression.

A. “Fixed Aminosterol Dose”

A “fixed aminosterol dose,” also referred to herein as a “fixed escalated aminosterol dose,” which will be therapeutically effective is determined for each subject by establishing a starting dose of an aminosterol composition and a threshold for improvement of depression and/or particular depression symptom. Following determining a starting dose of an aminosterol or salt or derivative thereof for a particular subject, the aminosterol dose is then progressively escalated by a consistent amount over consistent time intervals until the desired improvement in depression and/or particular depression symptom is achieved; this aminosterol dosage is the “fixed escalated aminosterol dosage” for that particular subject for that particular depression related symptom.

In exemplary embodiments, an orally administered aminosterol dose is escalated every about 3 to about 5 days by about 25 mg until the desired improvement is reached. Symptoms evaluated, along with tools for measuring symptom improvement, may be specifically described below.

This therapeutically effective “fixed aminosterol dose” is then maintained throughout treatment and/or prevention. Thus, even if the subject goes “off drug” and ceases taking the aminosterol composition, the same “fixed dose” is taken with no ramp up period following re-initiation of aminosterol treatment for depression and/or a depression related symptom.

Not to be bound by theory, it is believed that the aminosterol dose is dependent on the severity of nerve damage relating to the depression and/or a depression related symptom establishing the “fixed aminosterol dose” threshold—e.g., the dose may be related to the extent of nervous system damage in the patient's gut.

The aminosterol can be administered via any pharmaceutically acceptable means, such as by injection (e.g., IM, IV, or IP), oral, pulmonary, intranasal, etc. Preferably, the aminosterol is administered orally, intranasally, or a combination thereof.

Oral dosage of an aminosterol can range from about 1 to about 500 mg/day, or any amount in-between these two values. Other exemplary dosages of orally administered aminosterols include, but are not limited to, about 5, about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, about 205, about 210, about 215, about 220, about 225, about 230, about 235, about 240, about 245, about 250, about 255, about 260, about 265, about 270, about 275, about 280, about 285, about 290, about 295, about 300, about 305, about 310, about 315, about 320, about 325, about 330, about 335, about 340, about 345, about 350, about 355, about 360, about 365, about 370, about 375, about 380, about 385, about 390, about 395, about 400, about 405, about 410, about 415, about 420, about 425, about 430, about 435, about 440, about 445, about 450, about 455, about 460, about 465, about 470, about 475, about 480, about 485, about 490, about 495, or about 500 mg/day.

Intranasal dosages of an aminosterol are much lower than oral dosages of an aminosterol. Examples of such intranasal aminosterol low dosages include, but are not limited to, about 0.001 to about 6 mg/day, or any amount in-between these two values. For example, the low dosage of an intranasal administered aminosterol can be about 0.001, about 0.005, about 0.01, about 0.02, about 0.03, about 0.04, about 0.05, about 0.06, about 0.07, about 0.08, about 0.09, about 0.1, about 0.2, about 0.3, about 0.4, about 0.5, about 0.6, about 0.7, about 0.8, about 0.9, about 1, about 1.1, about 1.2, about 1.3, about 1.4, about 1.5, about 1.6, about 1.7, about 1.8, about 1.9, about 2, about 2.1, about 2.2, about 2.3, about 2.4, about 2.5, about 2.6, about 2.7, about 2.8, about 2.9, about 3, about 3.1, about 3.2, about 3.3, about 3.4, about 3.5, about 3.6, about 3.7, about 3.8, about 3.9, about 4, about 4.1, about 4.2, about 4.3, about 4.4, about 4.5, about 4.6, about 4.7, about 4.8, about 4.9, about 5, about 5.1, about 5.2, about 5.3, about 5.4, about 5.5, about 5.6, about 5.7, about 5.8, about 5.9, or about 6 mg/day.

For intranasal (IN) administration, it is contemplated that the aminosterol dosage may be selected such that it would not provide any pharmacological effect if administered by any other route and in addition, does not result in negative effects. For example, Aminosterol 1436 is known to have the pharmacological effects of a reduction in food intake and weight loss. Therefore, in the IN methods of the invention, if the aminosterol is Aminosterol 1436 or a salt or derivative thereof, then if the IN Aminosterol 1436 dosage is administered via another route, such as oral, IP, or IV, then the Aminosterol 1436 dosage will not result in a noticeable reduction in food intake or noticeable weight loss. Similarly, squalamine is known to produce the pharmacological effects of nausea, vomiting and/or reduced blood pressure. Thus, in the IN methods of the invention, if the aminosterol is squalamine or a salt or derivative thereof, then if the IN squalamine dosage is administered via another route, such as oral, IP, or IV, then the squalamine dosage will not result in noticeable nausea, vomiting, and/or a reduction in blood pressure. Suitable exemplary aminosterol dosages are described above.

Dose Escalation:

When determining a “fixed aminosterol dosage” for a particular subject, a subject is started at a lower dose and then the dose is escalated until a positive result is observed for the depression and/or depression related symptom. For example, determination of the fixed aminosterol dosage for treating depression and/or a depression related symptoms is shown in Example 1. Aminosterol doses can also be de-escalated (reduced) if any given aminosterol dose induces a persistent undesirable side effect, such as diarrhea, vomiting, or nausea.

The starting aminosterol dose is dependent on the severity of the symptom—e.g. for a subject experiencing severe depression, based on a baseline score of a depression test or tool that correlates with an assessment of depression, the starting oral aminosterol dose can be about 150 mg/day or greater. In contrast, for a subject having mild or moderate depression based on a baseline score of a medically recognized test or technique, for example those described herein, that correlates with an assessment of mild or moderate depression, the starting aminosterol dose can be about 75 mg/day or less. Thus, as an example, a subject experiencing mild moderate depression can be started at an aminosterol dosage of about 75 mg/day, whereas a subject experiencing severe depression can be started at an aminosterol dosage of about 150 mg/day. In some embodiments, the severity of the depression is reduced over a defined period of time, wherein the reduction is measured from one or more medically-recognized techniques selected from the group consisting of the Patient Health Questionnaire-9 (PHQ-9); the Beck Depression Inventory (BDI); Zung Self-Rating Depression Scale; Center for Epidemiologic Studies-Depression Scale (CES-D); and the Hamilton Rating Scale for Depression (HRSD). In an embodiment, the defined period of time is about 1 day to about 10 days, about 10 days to about 30 days, about 30 days to about 3 months, about 3 months to about 6 months, or about 6 months to about 12 months.

In other embodiments, a subject experiencing mild or moderate depression symptoms can be started at an oral aminosterol dosage of from about 10 mg/day to about 75 mg/day, or any amount in-between these values. The mild or moderate symptom may be mild or moderate depression based on a baseline score on a cognitive test or tool that correlates with an assessment of mild or moderate depression. For example, the starting oral aminosterol dosage for a subject having mild or moderate depression can be about 10, about 15, about 20, about 25, about 30, about 35, about 40, about 45, about 60, about 65, about 70, or about 75 mg/day. A fixed escalated oral aminosterol dose for a patient with mild or moderate depression is likely to range from about 5 mg up to about 350 mg/day, or any amount in-between these two values as described herein. In some embodiments, an oral fixed aminosterol dose, following dose escalation, is from about 50 to about 300 mg/daily, or from about 75 to about 275 mg/daily.

In yet further embodiments, when the subject is experiencing severe depression symptoms, for example, defined by a baseline score on a depression test or tool that correlates with severe depression, the subject can be started at an oral aminosterol dosage ranging from about 75 to about 300 mg/day, or any amount in-between these two values. In other embodiments, the starting oral aminosterol dosage for a subject with severe depression can be about 75, about 80, about 85, about 90, about 95, about 100, about 105, about 110, about 115, about 120, about 125, about 130, about 135, about 140, about 145, about 150 about 155, about 160, about 165, about 170, about 175 mg, about 180, about 185, about 190, about 195, about 200, about 205, about 210, about 215, about 220, about 225, about 230, about 235, about 240, about 245, about 250, about 255, about 260, about 265, about 270, about 275, about 280, about 285, about 290, about 295, or about 300 mg/day.

A “fixed escalated” oral aminosterol dose for a subject with severe depression is likely to range from about 75 up to about 500 mg/day.

Starting IN aminosterol dosages prior to dose escalation can be, for example, about 0.001 mg to about 3 mg/day, or any amount in-between these two values. For example, the starting aminosterol dosage for IN administration, prior to dose escalation, can be, for example, about 0.001, about 0.005, about 0.01, about 0.02, about 0.03, about 0.05, about 0.06, about 0.07, about 0.08, about 0.09, about 0.1, about 0.15, about 0.2, about 0.25, about 0.3, about 0.35, about 0.4, about 0.45, about 0.5, about 0.55, about 0.6, about 0.65, about 0.7, about 0.75, about 0.8, about 0.85, about 0.9, about 1.0, about 1.1, about 1.25, about 1.3, about 1.4, about 1.5, about 1.6, about 1.7, about 1.75, about 1.8, about 1.9, about 2.0, about 2.1, about 2.25, about 2.3, about 2.4, about 2.5, about 2.6, about 2.7, about 2.75, about 2.8, about 2.9, or about 3 mg.

In exemplary embodiments, the fixed dose aminosterol dose is given periodically as needed. For example, the fixed aminosterol dose can be administered once per day. The aminosterol dose can also be administered every other day, 2, 3, 4, 5, or 6× per week, once/week, or 2×/week. In another embodiment, the aminosterol dose can be administered every other week, or administered for a first period of time of administration, followed by a cessation of administration for a second period of time, followed by resuming administration upon recurrence of depression or a symptom of depression.

In some embodiments, the first period of time is about 1 day. In some embodiments, the first period of time is about 1 day to about 10 days. In some embodiments, the first period of time is about 10 days to about 30 days. In some embodiments, the first period of time is about 30 days to about 3 months. In some embodiments, the first period of time is about 3 months to about 6 months. In some embodiments, the first period of time is about 6 months to about 12 months.

In some embodiments, the second period of time is about 1 day. In some embodiments, the second period of time is about 1 day to about 10 days. In some embodiments, the second period of time is about 10 days to about 30 days. In some embodiments, the second period of time is about 30 days to about 3 months. In some embodiments, the second period of time is about 3 months to about 6 months. In some embodiments, the second period of time is about 6 months to about 12 months.

When calculating a fixed escalated aminosterol dose, the dose can be escalated following any suitable period of time. In one embodiment, the aminosterol dose is escalated every about 3 to about 7 days by a defined amount until a desired improvement is reached. In one embodiment, the aminosterol dose is escalated every about 3 to 5 days until a desired improvement is reached. For example, in some embodiments, the improvement in the depression-related symptom is measured using a clinical scale or tool and the improvement is about 3%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In other embodiments, the aminosterol dose can be escalated every about 1, about 2, about 3, about 4, about 5, about 6, about 7, about 8, about 9, about 10, about 11, about 12, about 13, or about 14 days. In other embodiments, the aminosterol dose can be escalated about 1×/week, about 2×/week, about every other week, or about 1×/month.

During dose escalation, the aminosterol dosage can be increased by a defined amount. For example, when the aminosterol is administered orally, the dose can be escalated in increments of about 5, about 10, about 15, about 20, about 25, about 30, about 35, about 40, about 45, or by about 50 mg. When the aminosterol is administered intranasally, then the dosage can be increased in increments of about, for example, about 0.1, about 0.2, about 0.25, about 0.3, about 0.35, about 0.4, about 0.45, about 0.5, about 0.55, about 0.6, about 0.65, about 0.7, about 0.75, about 0.8, about 0.85, about 0.9, about 0.95, about 1, about 1.1, about 1.2, about 1.3, about 1.4, about 1.5, about 1.6, about 1.7, about 1.8, about 1.9, or about 2 mg.

In exemplary embodiments, an orally administered aminosterol dose is escalated every about 3 to about 5 days by about 25 mg until an improvement of depression-related symptom is observed. The improvement of the depression related symptom may be measured using a clinical scale or tool, and the improvement is about 3%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In another embodiment, a fixed dose of an aminosterol can be varied plus or minus a defined amount to enable a modest reduction in a dose to eliminate adverse events, or a modest increase in a dose if clinical results suggest this is desirable—e.g., no or minimal adverse events and potential increased efficacy with a modest increase in dose. For example, in one embodiment a fixed aminosterol dose can be increased or decreased by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20%.

B. Depression and Depression-Related Symptoms to be Evaluated

The “fixed” dose of an aminosterol or a salt or derivative thereof is determined based upon the effect an escalated aminosterol dose has, over a period of time, on depression or a depression-related symptom. Measurable depression-related symptoms that can be evaluated include, for example (a) a symptom from the Hamilton Depression Rating Scale (HAM-D) selected from the group consisting of depressed mood, feelings of guilt, suicide, initial insomnia, middle of night insomnia, delayed insomnia, work and interests, retardation, agitation, psychic anxiety, somatic anxiety, gastrointestinal symptoms, general somatic symptoms, genital symptoms, hypochondriasis, weight loss, insight, diurnal variation, depersonalization and derealization, paranoid symptoms, and obsessional symptoms; (b) a symptom from the Montgomery-Asberg Depression Scale (MADRS) selected from the group consisting of apparent sadness, reported sadness, inner tension, reduced sleep, concentration difficulties, lassitude, inability to feel, pessimistic thoughts, and suicidal thoughts (c) a symptom from Beck's Depression Inventory (BDI) selected from the group consisting of sadness, outlook on the future, feelings of failure, satisfaction, guilt, feelings of being punished, disappointment with self, self-blame, suicidal ideation, crying frequency, prevalence of irritation, interest in others, ease of decision-making, self-image, ability to work, ease of sleep, tiredness, appetite, weight loss, preoccupation with health, and lack of libido; (d) apathy; (e) hopelessness; (f) loss of interest in hobbies; (g) sleep problem, sleep disorder, or sleep disturbance; (h) excessive hunger; (i) lack of appetite; (j) restlessness; (k) social isolation; (l) cognitive impairment; (m) weight loss; (n) weight gain; and (o) constipation.

The symptoms can be measured using a clinically recognized scale or tool, as detailed herein. The clinically recognized scale or tool may include, for example: The Hamilton Depression Rating Scale, Montgomery Asberg Depression Rating Scale, Raskin Depression Rating Scale, Beck Depression Inventory, Geriatric Depression Rating Scale, Patient Health Questionnaire, Primary Care Evaluation of Mental Disorders (PRIME-MD), The Clinically Useful Depression Outcome Scale (CUDOS), The Inventory of Depressive Symptomatology (IDS), Mood and Feelings Questionnaire (MFQ), Quick Inventory of Depressive Symptoms, Goldberg Test, Uniformed Parkinson's Disease Scale (UPDRS), Mini Mental State Examination (MMSE), Mini Mental Parkinson (MMP), Informant Questionnaire on Cognitive Decline in the Elderly (IQCODE), The 7-Minute Screen, Abbreviated Mental Test Score (AMTS), Cambridge Cognitive Examination (CAMCOG), Clock Drawing Test (CDT), General Practitioner Assessment of Cognition (GPCOG), Mini-Cog, Memory Impairment Screen (MIS), Montreal Cognitive Assessment (MoCA), Rowland Universal Dementia Assessment (RUDA), Self-Administered Gerocognitive Examination (SAGE), Short and Sweet Screening Instrument (SAS-SI), Short Blessed Test (SBT), St. Louis Mental Status (SLUMS), Short Portable Mental Status Questionnaire (SPMSQ), Short Test of Mental Status (STMS), Time and Change Test (T&C), Test Your Memory (TYM) test, and Addenbrooke's Cognitive Examination-Revised (ACER).

C. Aminosterols

U.S. Pat. No. 6,962,909, entitled “Treatment of neovascularization disorders with squalamine,” discloses various aminosterols, and this disclosure is specifically incorporated by reference with respect to its teaching of aminosterol compounds. Any aminosterol known in the art, including those described in U.S. Pat. No. 6,962,909, can be used in the disclosed compositions. In some embodiments, the aminosterol present in the compositions of the invention is Aminosterol 1436 or a salt or derivative thereof, squalamine or a salt or derivative thereof, or a combination thereof.

An aminosterol such as squalamine (ENT-01 in the examples) inhibits the formation of αS aggregates in vitro and in vivo, reverses motor dysfunction in the C. elegans αS model, and restores gastrointestinal motility in mouse models of PD.

Squalamine (ENT-01) has limited bioavailability in rats and dogs. Based on measurement of portal blood concentrations following oral dosing of radioactive ENT-01 to rat's absorption of ENT-01 from the intestine is low. As a consequence, the principal focus of safety is on local effects on the GIT. However, squalamine (ENT-01) appears to be well tolerated in both rats and dogs.

For instance, useful aminosterol compounds comprise a bile acid nucleus and a polyamine, attached at any position on the bile acid, such that the molecule exhibits a net positive charge contributed by the polyamine.

Thus, in some embodiments, the disclosed methods comprise administering a therapeutically effective amount of one or more aminosterols having the chemical structure of Formula I:

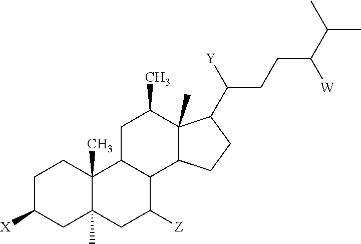

wherein,

W is 24S —OSO₃ or 24R—OSO₃;

X is β-H₂N—(CH₂)₄—NH—(CH₂)₃—NH— or 3α-H₂N—(CH₂)₄—NH—(CH₂)₃—NH—;

Y is 20R— CH₃; and

Z is 7α or 7β-OH.

In another embodiment of the invention, the aminosterol is one of the naturally occurring aminosterols (1-8) isolated from Squalus acanthias:

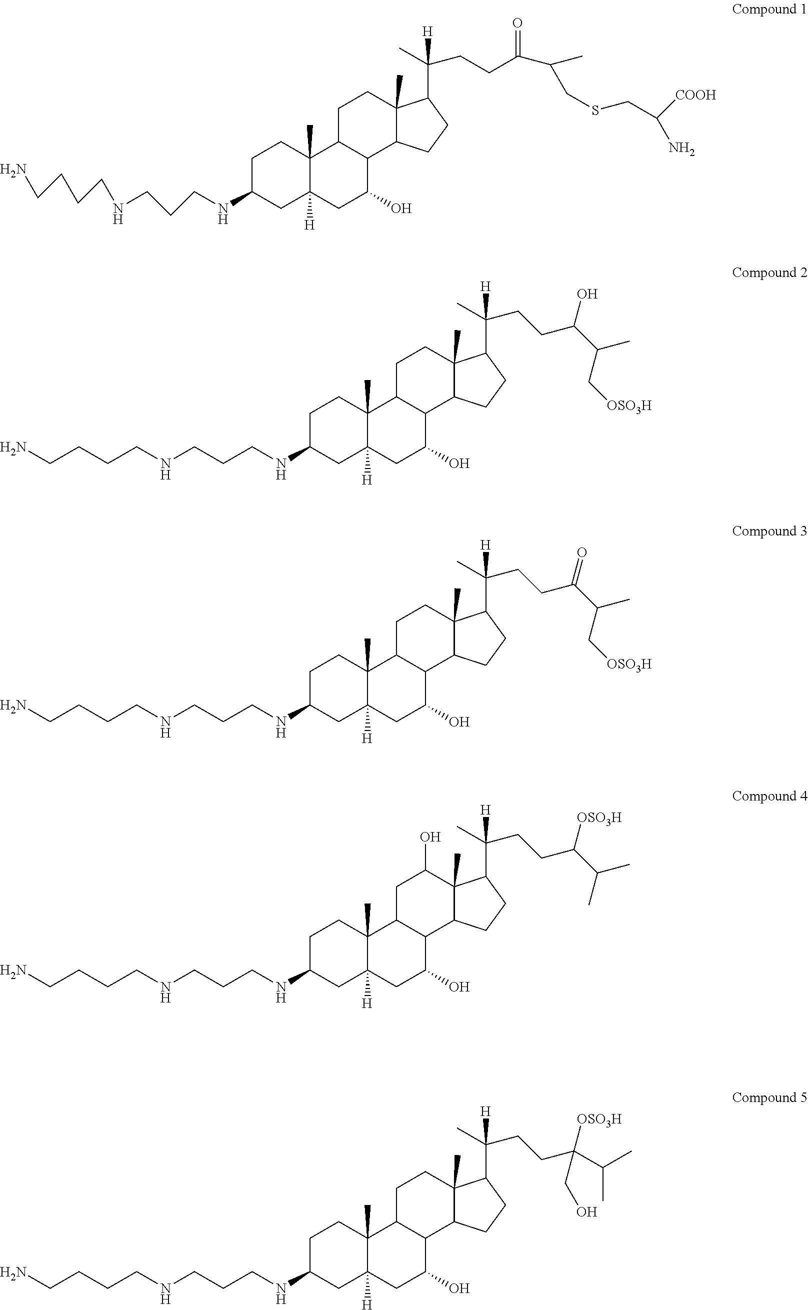

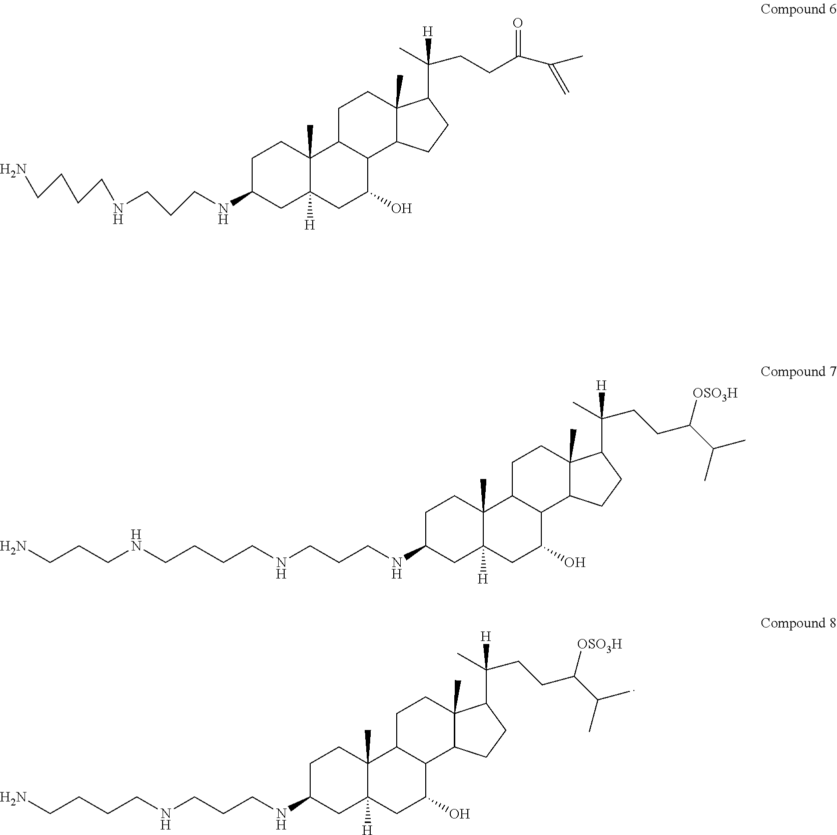

In one aspect of the invention, the aminosterol is Aminosterol 1436 or a salt or derivative thereof. In another embodiment the aminosterol is squalamine or a salt or derivative thereof.

Variants or derivatives of known aminosterols, such as squalamine, Aminosterol 1436, or an aminosterol isolated from Squalus acanthias, may be used in the disclosed compositions and methods.

In one embodiment, the aminosterol is squalamine, a squalamine isomer, a squalamine phosphate salt, aminosterol 1436, an aminosterol 1436 isomer, an aminosterol 1436 phosphate salt, or another naturally occurring aminosterol modified through medical chemistry to improve biodistribution, ease of administration, metabolic stability, or any combination thereof. In another embodiment, the aminosterol is modified to include one or more of the following: (1) substitutions of the sulfate by a sulfonate, phosphate, carboxylate, or other anionic moiety chosen to circumvent metabolic removal of the sulfate moiety and oxidation of the cholesterol side chain; (2) replacement of a hydroxyl group by a non-metabolizable polar substituent, such as a fluorine atom, to prevent its metabolic oxidation or conjugation; and (3) substitution of various ring hydrogen atoms to prevent oxidative or reductive metabolism of the steroid ring system.

In yet another embodiment, the aminosterol comprises a sterol nucleus and a polyamine, attached at any position on the sterol, such that the molecule exhibits a net charge of at least +1, the charge being contributed by the polyamine.

In yet another embodiment, the aminosterol comprises a bile acid nucleus and a polyamine, attached at any position on the bile acid, such that the molecule exhibits a net positive charge being contributed by the polyamine.

In some embodiments, the compositions used in the methods of the invention comprise: (a) at least one pharmaceutical grade aminosterol; and optionally (b) at least one phosphate selected from the group consisting of an inorganic phosphate, an inorganic pyrophosphate, and an organic phosphate. In some embodiments, the aminosterol is formulated as a weakly water soluble salt of the phosphate. In some embodiments, the phosphate is an inorganic polyphosphate, and the number of phosphates can range from about 3 (tripolyphosphate) to about 400, or any number in-between these two values. In other embodiments, the phosphate is an organic phosphate which comprises glycerol 2 phosphates.

In some embodiments, the aminosterol is selected from the group consisting of: (a) squalamine or a pharmaceutically acceptable salt or derivative thereof; (b) a squalamine isomer; (c) a squalamine phosphate salt; (d) Aminosterol 1436 or a pharmaceutically acceptable salt or derivative thereof; (e) an isomer of aminosterol 1436; (f) an aminosterol 1436 phosphate salt, (g) a synthetic aminosterol; (h) an aminosterol comprising a sterol or bile acid nucleus and a polyamine, attached at any position on the sterol or bile acid, such that the molecule exhibits a net charge of at least +1, the charge being contributed by the polyamine; (i) an aminosterol which is a derivative of squalamine modified through medical chemistry to improve biodistribution, ease of administration, metabolic stability, or any combination thereof; (j) an aminosterol modified to include one or more of the following: (i) substitutions of the sulfate by a sulfonate, phosphate, carboxylate, or other anionic moiety chosen to circumvent metabolic removal of the sulfate moiety and oxidation of the cholesterol side chain; (ii) replacement of a hydroxyl group by a non-metabolizable polar substituent, such as a fluorine atom, to prevent its metabolic oxidation or conjugation; and (iii) substitution of various ring hydrogen atoms to prevent oxidative or reductive metabolism of the steroid ring system; (g) an aminosterol that can inhibit the formation of actin stress fibers in endothelial cells stimulated by a ligand known to induce stress fiber formation, having the chemical structure of Formula I (above); or (h) any combination thereof.

In some embodiments, the methods of the invention can employ a formulation of Aminosterol 1436 or squalamine as an insoluble salt of phosphate, polyphosphate, or an organic phosphate ester.

Any pharmaceutically acceptable salt of an aminosterol can be used in the compositions and methods of the invention. For example, a phosphate salt or buffer, free base, succinate, phosphate, mesylate or other salt form associated with low mucosal irritation can be utilized in the methods and compositions of the invention.

D. Routes of Administration

It is appreciated that the “fixed dose” disclosed herein can be administered via any suitable route of administration, including but not limited to oral or intranasal delivery, injection (IP, IV, or IM) or a combination thereof.

Further, co-administration of the “fixed dose” with injectable (e.g., IP, IV, IM) aminosterol formulations is also contemplated herein. For injectable dosage forms, the dosage form can comprise an aminosterol at a dosage of, for example, about 0.1 to about 20 mg/kg body weight. In other embodiments, the effective daily dosing amount is about 0.1, about 0.5, about 1, about 2, about 3, about 4, about 5, about 6, about 7, about 8, about 9, about 10, about 11, about 12, about 13, about 14, about 15, about 16, about 17, about 18, about 19, or about 20 mg/kg body weight.

The invention also encompasses methods of treatment using a combination of an aminosterol composition administered via one route, e.g., oral, with a second aminosterol composition, comprising the same or a different aminosterol, administered via a different route, e.g., intranasal. For example, in a method of the invention, squalamine can be administered orally and aminosterol 1436 can be administered IN.

E. Dosing Period

The pharmaceutical composition comprising an aminosterol or a derivative or salt thereof can be administered for any suitable period of time, including as a maintenance dose for a prolonged period of time. Dosing can be done on an as needed basis using any pharmaceutically acceptable dosing regimen. Aminosterol dosing can be no more than 1× per day, once every other day, once every three days, once every four days, once every five days, once every six days, once a week, or divided over multiple periods of time during a given day (e.g., twice daily).

In other embodiments, the composition can be administered: (1) as a single dose, or as multiple doses over a period of time; (2) at a maintenance dose for an indefinite period of time; (3) once, twice or multiple times; (4) daily, every other day, every 3 days, weekly, or monthly; (5) for a period of time such as about 1, about 2, about 3, or about 4 weeks, about 1, about 2, about 3, about 4, about 5, about 6, about 7, about 8, about 9, about 10, about 11, or about 12 months, about 1 year, about 1.5 years, about 2, about 2.5, about 3, about 3.5, about 4, about 4.5, about 5, about 5.5, about 6, about 6.5, about 7, about 7.5, about 8, about 8.5, about 9, about 9.5, about 10, about 10.5, about 11, about 11.5, about 12, about 12.5, about 13, about 13.5, about 14, about 14.5, about 15, about 15.5, about 16, about 16.5, about 17, about 17.5, about 18, about 18.5, about 19, about 19.5, about 20, about 20.5, about 21, about 21.5, about 22, about 22.5, about 23, about 23.5, about 24, about 24.5, or about 25 years, or (6) any combination of these parameters, such as daily administration for 6 months, weekly administration for 1 or more years, etc.

Yet another exemplary dosing regimen includes periodic dosing, where an effective dose can be delivered once every about 1, about 2, about 3, about 4, about 5, about 6 days, or once weekly.

In a preferred embodiment, the aminosterol dose is taken in the morning, i.e. on an empty stomach preferably within about two hours of waking up and may be followed by a period without food, such as for example about 60 to about 90 minutes. In other embodiments, the aminosterol dose is taken within about 15 min, about 30 min, about 45 min, about 1 hr, about 1.25 hrs, about 1.5 hrs, about 1.75 hrs, about 2 hrs, about 2.25 hrs, about 2.5 hrs, about 2.75 hrs, about 3 hrs, about 3.25 hrs, about 3.5 hrs, about 3.75 hrs, or about 4 hrs within waking up. In yet further embodiments, the aminosterol dose is followed by about period without food, wherein the period is at least about 30 min, about 45 mins, about 60 mins, about 1.25 hrs, about 1.5 hrs, about 1.75 hrs, or about 2 hrs.

Not to be bound by theory, it is believed that since aminosterols have an impact on circadian rhythms, likely due to ENS signaling thereof, taking the aminosterol dose in the morning enables the synchronization of all the autonomic physiological functions occurring during the day. In other embodiments of the invention, the aminosterol dosage is taken within about 15 mins, about 30 mins, about 45 mins, about 1 hour, about 1.25 hrs, about 1.5 hrs, about 1.75 hrs, about 2 hrs, about 2.25 hrs, about 2.5 hrs, about 2.75 hrs, about 3 hrs, about 3.25 hrs, about 3.5 hrs, about 3.75 hrs, or about 4 hrs of waking up. In addition, in other embodiments of the invention, following the aminosterol dosage the subject has a period of about 15 mins, about 30 mins, about 45 mins, about 1 hours, about 1.25 hrs, about 1.5 hrs, about 1.75 hrs, about 2 hrs, about 2.25 hrs, about 2.5 hrs, about 2.75 hrs, or about 3 hours without food.

In any embodiment herein, each period of time is independently selected from the group consisting of about 1 day to about 10 days, about 10 days to about 30 days, about 30 days to about 3 months, about 3 months to about 6 months, about 6 months to about 12 months and about greater than 12 months.

F. Composition Components

In some embodiments, a pharmaceutical composition disclosed herein comprises one or more pharmaceutically acceptable carriers, such as an aqueous carrier, buffer, and/or diluent.

In some embodiments, a pharmaceutical composition disclosed herein further comprises a simple polyol compound, such as glycerin. Other examples of polyol compounds include sugar alcohols. In some embodiments, a pharmaceutical composition disclosed herein comprises an aqueous carrier and glycerin at about a 2:1 ratio.

The formulations may conveniently be presented in unit dosage form and may be prepared by any of the methods well known in the art of pharmacy. An exemplary oral dosage form is a tablet or capsule. An exemplary intranasal dosage form is a liquid or powder nasal spray. A nasal spray is designed to deliver drug to the upper nasal cavity, and can be a liquid or powder formulation, and in a dosage form such as an aerosol, liquid spray, or powder.

The aminosterol may be combined or coordinately administered with a suitable carrier or vehicle depending on the route of administration. As used herein, the term “carrier” means a pharmaceutically acceptable solid or liquid filler, diluent or encapsulating material. A water-containing liquid carrier can comprise pharmaceutically acceptable additives such as acidifying agents, alkalizing agents, antimicrobial preservatives, antioxidants, buffering agents, chelating agents, complexing agents, solubilizing agents, humectants, solvents, suspending and/or viscosity-increasing agents, tonicity agents, wetting agents or other biocompatible materials. A tabulation of ingredients listed by the above categories can be found in the U.S. Pharmacopeia National Formulary, 1857-1859, and (1990). Some examples of the materials which can serve as pharmaceutically acceptable carriers are sugars, such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols, such as propylene glycol; polyols such as glycerin, sorbitol, mannitol and polyethylene glycol; esters such as ethyl oleate and ethyl laurate; agar; buffering agents such as magnesium hydroxide and aluminum hydroxide; alginic acid; pyrogen free water; isotonic saline; Ringer's solution, ethyl alcohol and phosphate buffer solutions, as well as other nontoxic compatible substances used in pharmaceutical formulations. Wetting agents, emulsifiers and lubricants such as sodium lauryl sulfate and magnesium stearate, as well as coloring agents, release agents, coating agents, sweetening, flavoring and perfuming agents, preservatives and antioxidants can also be present in the compositions, according to the desires of the formulator. Examples of pharmaceutically acceptable antioxidants include water soluble antioxidants such as ascorbic acid, cysteine hydrochloride, sodium bisulfite, sodium metabisulfite, sodium sulfite and the like; oil-soluble antioxidants such as ascorbyl palmitate, butylated hydroxyanisole (BHA), butylated hydroxytoluene (BHT), lecithin, propyl gallate, alpha-tocopherol and the like; and metal-chelating agents such as citric acid, ethylenediamine tetraacetic acid (EDTA), sorbitol, tartaric acid, phosphoric acid and the like.

Pharmaceutical compositions according to the invention may also comprise one or more binding agents, filling agents, lubricating agents, suspending agents, sweeteners, flavoring agents, preservatives, buffers, wetting agents, disintegrants, effervescent agents, and other excipients. Such excipients are known in the art. Examples of filling agents include lactose monohydrate, lactose anhydrous, and various starches; examples of binding agents include various celluloses and cross-linked polyvinylpyrrolidone, microcrystalline cellulose, such as Avicel® PH101 and Avicel® PH102, microcrystalline cellulose, and silicified microcrystalline cellulose (ProSolv SMCC™). Suitable lubricants, including agents that act on the flowability of the powder to be compressed, may include colloidal silicon dioxide, such as Aerosil® 200, talc, stearic acid, magnesium stearate, calcium stearate, and silica gel. Examples of sweeteners may include any natural or artificial sweetener, such as sucrose, xylitol, sodium saccharin, cyclamate, aspartame, and acesulfame. Examples of flavoring agents are Magnasweet® (trademark of MAFCO), bubble gum flavor, and fruit flavors, and the like. Examples of preservatives include potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quaternary compounds such as benzalkonium chloride.

Any pharmaceutical used for therapeutic administration can be sterile. Sterility is readily accomplished by for example filtration through sterile filtration membranes (e.g., 0.2 micron membranes). Any pharmaceutically acceptable sterility method can be used in the compositions of the invention.

The pharmaceutical composition comprising an aminosterol derivatives or salts thereof will be formulated and dosed in a fashion consistent with good medical practice, taking into account the clinical condition of the individual patient, the method of administration, the scheduling of administration, and other factors known to practitioners.

G. Kits

Aminosterol formulations or compositions of the invention may be packaged together with, or included in a kit along with instructions or a package insert. Such instructions or package inserts may address recommended storage conditions, such as time, temperature and light, taking into account the shelf-life of the aminosterol or derivatives or salts thereof. Such instructions or package inserts may also address the particular advantages of the aminosterol or derivatives or salts thereof, such as the ease of storage for formulations that may require use in the field, outside of controlled hospital, clinic or office conditions.

The invention also provides a pharmaceutical pack or kit comprising one or more containers filled with one or more aminosterol pharmaceutical compositions disclosed herein. The kits may include, for instance, containers filled with an appropriate amount of an aminosterol pharmaceutical composition, either as a powder, a tablet, to be dissolved, or as a sterile solution. Associated with such container(s) can be a notice in the form prescribed by a governmental agency regulating the manufacture, use or sale of pharmaceuticals or biological products, which notice reflects approval by the agency of manufacture, use or sale for human administration. In addition, the aminosterol or a derivative or salt thereof may be employed in conjunction with other therapeutic compounds.

In other aspects, a kit comprising a nasal spray device as described herein is disclosed. In one aspect, the kit may comprise one or more devices as disclosed herein, comprising a disclosed low dose aminosterol composition, wherein the device is sealed within a container sufficient to protect the device from atmospheric influences. The container may be, for example, a foil, or plastic pouch, particularly a foil pouch, or heat sealed foil pouch. Suitable containers sufficient to adequately protect the device will be readily appreciated by one of skill in the art.

In one aspect, the kit may comprise one or more devices as disclosed herein, wherein the device may be sealed within a first protective packaging, or a second protective packaging, or a third protective packaging, that protects the physical integrity of the product. One or more of the first, second, or third protective packaging may comprise a foil pouch. The kit may further comprise instructions for use of the device. In one aspect, the kit contains two or more devices.

In one aspect, the kit may comprise a device as disclosed herein, and may further comprise instructions for use. In one aspect, the instructions may comprise visual aid/pictorial and/or written directions to an administrator of the device.

H. Patient Populations

The disclosed compositions can be used to treat a range of subjects, including human and non-human animals, including mammals, as well as immature and mature animals, including human children and adults. The human subject to be treated can be an infant, toddler, school-aged child, teenager, young adult, adult, or elderly patient.

In embodiments disclosed herein relating to prevention and/or treatment, particular patient populations may be selected based on being “at risk for” the development of one or more disorders correlated with depression. For example, genetic markers of PD (e.g. SNCA (PARK1), UCHL1 (PARK 5), and LRRK2 (PARK8)) or family history may be used as signs to identify subjects likely to develop PD and experience depression. Thus, in some embodiments relating to disorders for which certain genetic or hereditary signs are known, prevention may involve first identifying a patient population based on one of the signs. Alternatively, certain symptoms are considered early signs of particular disorders. For example, constipation is considered an early sign of PD. Thus, in some embodiments, relating to PD a patient population may be selected for being “at risk” for developing PD based on age and experiencing constipation. An exemplary population is adults between the ages of about 50 and about 60 experiencing constipation characterized by less than 3 bowel movements per week. These patients can be targeted and monitored for prevention of PD onset. Further genetic or hereditary signs may be used to refine the patient population.

### IV. Methods of Treating Depression and/or a Depression-Related Condition or Disease with a “Fixed Dose” of Aminosterol

Aspects of this disclosure relate to methods of treating, preventing, and/or delaying the onset or progression of depression and/or a depression related condition by administration of a “fixed dose” of an aminosterol as disclosed herein. The depression can be correlated with abnormal α-synuclein (αS) pathology. Alternatively, the depression can be correlated dysfunctional DA neurotransmission, also known as dopaminergic dysfunction.

This disclosure provides a detailed protocol for determining a “fixed dose” based on improvement of one symptom associated with Parkinson's disease (PD), e.g., depression and depression-related symptoms as measured by clinically recognized scales and tools.

As dopaminergic activity distinguishes PD from other neurodegenerative disorders and these data relate to symptoms that in some embodiments, not relate to this distinguishing feature, this dosing regime is believed to be extrapolatable both depression per se and depression related symptoms.

Not to be bound by theory, it is believed that establishing a patient-specific “fixed dose” based on obtaining a threshold improvement in any of the depression-related symptoms described herein will successfully treat depression and/or depression related symptoms. Further, to the extent that these symptoms are tied to an underlying disorder, administration of the therapeutically effective fixed dose is also believed to offer a means of treating, preventing, and/or delaying onset of the underlying disorder or disease causing the depression or depression-related symptom.

A. Depression

As used herein “depression” refers to a common but serious mood disorder. It causes severe symptoms that affect how one feels, thinks, and handles daily activities, such as sleeping, eating, or working. To be diagnosed with depression, the symptoms must be present for at least two weeks.

There are different types of depression. Persistent depressive disorder (also called dysthymia) is a depressed mood that lasts for at least two years in a subject. A person diagnosed with persistent depressive disorder may have episodes of major depression along with periods of less severe symptoms, but symptoms must last for two years to be considered persistent depressive disorder.

Postpartum depression is much more serious than the “baby blues” (relatively mild depressive and anxiety symptoms that typically clear within two weeks after delivery) that many women experience after giving birth. Women with postpartum depression experience full-blown major depression during pregnancy or after delivery (postpartum depression). The feelings of extreme sadness, anxiety, and exhaustion that accompany postpartum depression may make it difficult for these new mothers to complete daily care activities for themselves and/or for their babies.

Psychotic depression occurs when a subject has severe depression plus some form of psychosis, such as having disturbing false fixed beliefs (delusions) or hearing or seeing upsetting things that others cannot hear or see (hallucinations). The psychotic symptoms typically have a depressive “theme,” such as delusions of guilt, poverty, or illness.

Seasonal affective disorder is characterized by the onset of depression during the winter months, when there is less natural sunlight. This depression generally lifts during spring and summer. Winter depression, typically accompanied by social withdrawal, increased sleep, and weight gain, predictably returns every year in seasonal affective disorder.

Bipolar disorder is different from depression. A subject with bipolar disorder experiences episodes of extremely low moods that meet the criteria for major depression (called “bipolar depression”). A person with bipolar disorder also experiences extreme high—euphoric or irritable—moods called “mania” or a less severe form called “hypomania.”

Examples of other types of depressive disorders newly added to the diagnostic classification of Diagnostic and Statistical Manual of Mental Disorders, Fifth Edition (DSM-5) include disruptive mood dysregulation disorder (diagnosed in children and adolescents) and premenstrual dysphoric disorder (PMDD).

In diagnosing depression, clinicians may first conduct a CT or MRI scan of the brain to rule out diseases such as cancer. Questionnaires are used to completed the diagnosis. One example of a screening test is a two-part questionnaire that has been shown to be highly reliable in identifying the likelihood of depression. The clinician will ask the patient the following two questions: “During the past month, have you been bothered by feeling down, depressed, or hopeless” and “during the past month, have you been bothered by little interest or pleasure in doing things?” Written tests that measure severity of depression symptoms are also used, these include the Patient Health Questionnaire-9 (PHQ-9), a 9-item self-administered diagnostic screening and severity tool based on current diagnostic criteria for major depression; the Beck Depression Inventory (BDI), a 21-question multiple-choice self-report that measures the severity of depression symptoms and feelings; Zung Self-Rating Depression Scale, a short survey that measures the level of depression, ranging from normal to severely depressed; Center for Epidemiologic Studies-Depression Scale (CES-D), an instrument that allows patients to evaluate their feelings, behavior, and outlook from the previous week; and the Hamilton Rating Scale for Depression (HRSD), also known as the Hamilton Depression Rating Scale (HDRS) or abbreviated to HAM-D, a multiple choice questionnaire that doctors may use to rate the severity of a patient's depression.

Measurable depression related symptoms that can be evaluated include a symptom from the Hamilton Depression Rating Scale (HAM-D) selected from the group consisting of depressed mood, feelings of guilt, suicide, initial insomnia, middle of night insomnia, delayed insomnia, work and interests, retardation, agitation, psychic anxiety, somatic anxiety, gastrointestinal symptoms, general somatic symptoms, genital symptoms, hypochondriasis, weight loss, insight, diurnal variation, depersonalization and derealization, paranoid symptoms, and obsessional symptoms; (b) a symptom from the Montgomery-Asberg Depression Scale (MADRS) selected from the group consisting of apparent sadness, reported sadness, inner tension, reduced sleep, concentration difficulties, lassitude, inability to feel, pessimistic thoughts, and suicidal thoughts (c) a symptom from Beck's Depression Inventory (BDI) selected from the group consisting of sadness, outlook on the future, feelings of failure, satisfaction, guilt, feelings of being punished, disappointment with self, self-blame, suicidal ideation, crying frequency, prevalence of irritation, interest in others, ease of decision-making, self-image, ability to work, ease of sleep, tiredness, appetite, weight loss, preoccupation with health, and lack of libido; (d) apathy; (e) hopelessness; (f) loss of interest in hobbies; (g) sleep problem, sleep disorder, or sleep disturbance; (h) excessive hunger; (i) lack of appetite; (j) restlessness; (k) social isolation; (l) cognitive impairment; (m) weight loss; (n) weight gain; and (o) constipation.

Potentially all of these symptoms or characteristics can be positively impacted by the methods of the invention. Further, assessments of these characteristics can be done using clinically recognized scales, as described herein.

In one embodiment, the method results in a positive impact or improvement in depression or a depression-related symptom, or an underlying disease or disorder correlated with depression, measured using a clinical scale or tool, and the improvement is about 3%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

The questionnaires discussed herein can be used to evaluate depression and related symptoms in the claimed methods. For example, the Beck Depression Inventory rates patients using 21 questions, each answer being scored on a scale value of 0 to 3. Each question had a set of at least four possible responses, ranging in intensity. Questions relate to how the subject has been feeling in the week previous to the questionnaire. For example, the subject will be asked to select one option 0-3 which include: (0) I do not feel sad; (1) I feel ad; (2) I am sad all the time and I can't snap out of it; and (3) I am so sad or unhappy that I can't stand it. Higher total scores indicate more severe depressive symptoms. The standardized cutoffs used differ from the original and are: 0-13: minimal depression; 14-19: mild depression; 20-28: moderate depression; and 29-63: severe depression.

The Montgomery-Asberg Depression Rating Scale (MADRS) (Montgomery et al., 1979) is another questionnaire that may be used. Higher MADRS score indicates more severe depression, and each item yields a score of 0 to 6. The overall score ranges from 0 to 60. The questionnaire includes questions on the following symptoms 1. Apparent sadness 2. Reported sadness 3. Inner tension 4. Reduced sleep 5. Reduced appetite 6. Concentration difficulties 7. Lassitude 8. Inability to feel 9. Pessimistic thoughts 10. Suicidal thoughts. Usual cutoff points are: 0 to 6 indicates normal/symptom absent, 7 to 19 indicates mild depression, 20 to 34 indicates moderate depression and >34 indicates severe depression.

As detailed in Example 1 depression and the improvement following aminosterol treatment in patients were assessed using the Beck Depression Inventory (BDI-II) (Steer et al. 2000) and Unified Parkinson's Disease Rating Scale (UPDRS) as clinical tools.

Example 1, includes evaluation of symptoms including depression, in patients administered an aminosterol. Assessments were made at baseline and at the end of the fixed dose and washout periods for Example 1, and an analysis was done with respect to the cognition symptoms. The results showed that the total UPDRS score was 64.4 at baseline, 60.6 at the end of the fixed dose period and 55.7 at the end of the wash-out period (a 13.5% improvement). Part 1 of the UPDRS (which includes section 1.3, Depressed Mood) had a mean baseline score of 11.6, a fixed aminosterol dose mean score of 10.6, and a wash-out mean score of 9.5, demonstrating an almost 20% improvement (UPDRS Depressed Mood is rated from 1=slight depression (includes episodes of depressed mood lasting less than a day and not interfering with life) to 4=severe depression, so lower scores correlate with lower levels of depression). In addition, Beck Depression Index II (BDI-II) improved from 10.9 at baseline to 9.9 during treatment and to 8.7 during wash-out. Unlike stool-related indices, the improvement in many CNS symptoms persisted during wash-out.

Examples of conditions and/or symptoms associated with depression correlated with abnormal αS pathology, and/or dysfunctional DA neurotransmission, also known as dopaminergic dysfunction, are described herein, such as in Section 1.B. above, as well as below.

Examples of conditions associated with abnormal αS pathology, and/or dopaminergic dysfunction, correlated with depression include, but are not limited to, synucleopathies, neurodiseases, psychological and/or behavior disorders, cerebral and general ischemic disorderes, and/or disorders or conditions such as AD, PD, dementia with Lewy bodies (DLB), multiple system atrophy (MSA), Huntington's Disease, Multiple Sclerosis (MS), Amyotorphic Lateral Sclerosis (ALS), schizophrenia, Friedreich's ataxia, vascular dementia, spinal muscular atrophy, supranuclear palsy, fronto temperal dementia (FTD), progressive supranuclear palsy, Guadeloupian Parkinsonism, spinocerebellar ataxia, autism, stroke, traumatic brain injury, sleep disorders such as REM sleep behavior disorder (RBD), depression, down syndrome, Gaucher's disease (GD), Krabbe's disease (KD), lysosomal conditions affecting glycosphingolipid metabolism, ADHD, agitation, anxiety, delirium, irritability, illusion and delusions, amnesia, apathy, bipolar disorder, disinhibition, aberrant motor and obsessive-compulsive behaviors, addiction, cerebral palsy, epilepsy, and major depressive disorder.

1. Neurodegenerative Diseases and Neurological Diseases Associated with Neural Cell Death

The methods and compositions of the invention may also be useful in treating, preventing, and/or slowing the onset or progression of depression correlated with abnormal αS pathology, and/or dysfunctional DA neurotransmission, wherein the underlying disease or disorder is a neurodegenerative disease or neurological disorder. Examples of such neurodegenerative diseases or neurological disorders include, but are not limited to, PD, AD, LBD, FTD, supranuclear palsy, MSA, Parkinsonism, ALS, Huntington's Disease, schizophrenia, Friedreich's ataxia, multiple sclerosis (MS), spinal muscular atrophy, progressive nuclear palsy, degenerative processes associated with aging, dementia of aging, Guadeloupian Parkinsonism, spinocerebellar ataxia, and vascular dementia.

In addition, the methods and compositions of the invention may also be useful in treating, preventing, and/or slowing the onset or progression of depression correlated with abnormal αS pathology, and/or dysfunctional DA neurotransmission, wherein the underlying disease or disorder is a neurological disease associated with neural cell death and/or related symptoms of neural cell death such as septic shock, intracerebral bleeding, subarachnoidal hemorrhage, multiinfarct dementia, inflammatory diseases, neurotrauma, peripheral neuropathies, polyneuropathies, epilepsies, schizophrenia, depression, metabolic encephalopathies, or infections of the central nervous system.

A variety of neuroimaging techniques may be useful for the early diagnosis and/or measurement of progression of neurodegenerative disorders correlated with depression. Examples of such techniques include but are not limited to neuroimaging, functional MRI, structural MRI, diffusion tensor imaging (DTI) (including for example diffusion tensor measures of anatomical connectivity), [18F]fluorodeoxyglucose (FDG) PET, agents that label amyloid, [18F]F-dopa PET, radiotracer imaging, volumetric analysis of regional tissue loss, specific imaging markers of abnormal protein deposition (e.g., for depression associated disease such as AD and PD), multimodal imaging, and biomarker analysis (Jon Stoessl, 2012). Combinations of these techniques can also be used to measure disease progression. For example, structural MRI can be used to measure atrophy of the hippocampus and entorhinal cortex in AD, as well as involvement of the lateral parietal, posterior superior temporal and medial posterior cingulate cortices. DTI can be used to show abnormal white matter in the parietal lobes of patients with dementia with Lewy bodies (DLB) as compared to AD. Functional MRI may reveal reduced frontal but increased cerebellar activation during performance of a working memory task in FTD compared to AD. In another example, [18F]fluorodeoxyglucose (FDG) PET can show reduced glucose metabolism in parietaltemporal cortex in AD. An electroencephalogram (EEG) can be used as a biomarker for the presence and progression of a neurodegenerative disease.

i. Parkinson's Disease

PD is the second most common age-related neurodegenerative disease after AD (Reeve et al. 2014). PD affects over 1% of the population over the age of 60, which in the US equates to over 500,000 individuals, while in individuals over the age of 85 this prevalence reaches 5%, highlighting the impact that advancing age has on the risk of developing this condition. Id.

While motor symptoms are still required for a diagnosis of PD (Hughes et al. 1992), non-motor symptoms represent a greater therapeutic challenge (Zahodne et al. 2012). These symptoms include cognitive dysfunction (Auyeung et al. 2012), as well as constipation (Ondo et al. 2012; Lin et al. 2014), disturbances in sleep architecture (Ondo et al. 2001; Gjerstad et al. 2006), hallucinations (Friedman et al. 2016; Diederich et al. 2006), REM behavior disorder (RBD) and depression (Aarsland et al. 2007), all of which result from impaired function of neural pathways not restored by replacement of dopamine. In fact, long-term institutionalization, caregiver burden and decrease in life expectancy correlate more significantly with the severity of these symptoms than with motor symptoms (Goetz et al. 1995).

PD is a progressive neurodegenerative disorder caused by accumulation of the protein αS within the ENS, autonomic nerves and brain (Braak et al. 2003). In 2003, Braak proposed that PD begins within the GI tract caused when neurotoxic aggregates of αS form within the ENS, evidenced clinically by the appearance of constipation in a majority of people with PD many years before the onset of motor symptoms. A recent study in rats has demonstrated movement of aggregates of αS from the ENS to the CNS via the vagus and other afferent nerves. Neurotoxic aggregates accumulated progressively within the brainstem and then dispersed rostrally to structures within the diencephalon, eventually reaching the cerebral hemispheres.

PD is defined as a synucleinopathy, and synuclein deposition remains the main final arbiter of diagnosis. Additionally, patients with dementia and Lewy bodies are considered as having PD if they meet clinical disease criteria. Imaging (e.g., MRI, single photon emission computed tomography [SPECT], and positron emission tomography [PET]) allows in vivo brain imaging of structural, functional, and molecular changes in PD patients.

There has been research in the last few years identifying particular markers or combinations of markers that are used for probabilistic estimates of prodromal PD. Researchers have identified a timeline of symptoms indicative of prodromal PD and predictive of PD. The presence of each contributes to an estimate of the likelihood of prodromal PD. Some have been adopted for identification of prodromal PD. Other studies use a combination of symptoms and imaging (e.g., hyposmia combined with dopamine receptor imaging has been found to have a high predictive value). In another example, REM sleep behavior disorder (SBD), constipation, and hyposmia were found to be individually common but to rarely co-occur in individuals without PD, leading to a high predictive value for PD. Thus, patient populations having RBD, constipation, and/or hyposmia are considered at risk for developing PD.

Data described in Example 1 shows remarkable improvement in a wide variety of symptoms correlated with PD, including a significant and positive effect on depression. The study demonstrates that administration of an aminosterol can displace αS from membranes in vitro and reduce the formation of neurotoxic αS aggregates in vivo, thereby improving depression. The study is the first proof of concept demonstration that directly targeting αS pharmacologically can achieve beneficial GI, autonomic and CNS responses to improve depression in patients suffering from neurodiseases such as PD. These results demonstrate that the ENS in PD is not irreversibly damaged and can be restored to normal function.

As described in Example 1, CNS symptoms were evaluated at baseline and at the end of the fixed dose period and the wash-out period (Table 12). Moreover, the improvement in many CNS symptoms persisted during wash-out. The results of treatment were dramatic: MMSE (cognitive ability) improved from 28.4 at baseline to 28.7 during treatment and to 29.3 during wash-out. Other symptoms evaluated and showing improvement included:

(1) Total UPDRS score was 64.4 at baseline, 60.6 at the end of the fixed dose period and 55.7 at the end of the wash-out period; similarly, the motor component of the UPDRS improved from 35.3 at baseline to 33.3 at the end of fixed dose to 30.2 at the end of wash-out. The UPDRS score, a global assessment of motor and non-motor symptoms, showed significant improvement. Improvement was also seen in the motor component. The improvement in the motor component is unlikely to be due to improved gastric motility and increased absorption of dopaminergic medications, since improvement persisted during the 2-week wash-out period, i.e., in the absence of study drug (Table 12).

(2) BDI-II (depression) decreased from 10.9 at baseline to 9.9 during treatment and 8.7 at wash-out.

(3) PDHQ (hallucinations) improved from 1.3 at baseline to 1.8 during treatment and 0.9 during wash-out. Hallucinations were reported by 5 patients at baseline and delusions in 1 patient. Both hallucinations and delusions improved or disappeared in 5 of 6 patients during treatment and did not return for 4 weeks following discontinuation of aminosterol treatment in 1 patient and 2 weeks in another. In one patient the hallucinations disappeared at 100 mg, despite not having reached the colonic prokinetic dose at 175 mg.

(4) Improvements were seen in REM-behavior disorder (RBD) and sleep. RBD and total sleep time also improved progressively in a dose-dependent manner. The frequency of arm or leg thrashing reported in the sleep diary diminished progressively from 2.2 episodes/week at baseline to 0 at maximal dose. Total sleep time increased progressively from 7.1 hours at baseline to 8.4 hours at 250 mg and was consistently higher than baseline beyond 125 mg (FIGS. 6-8).

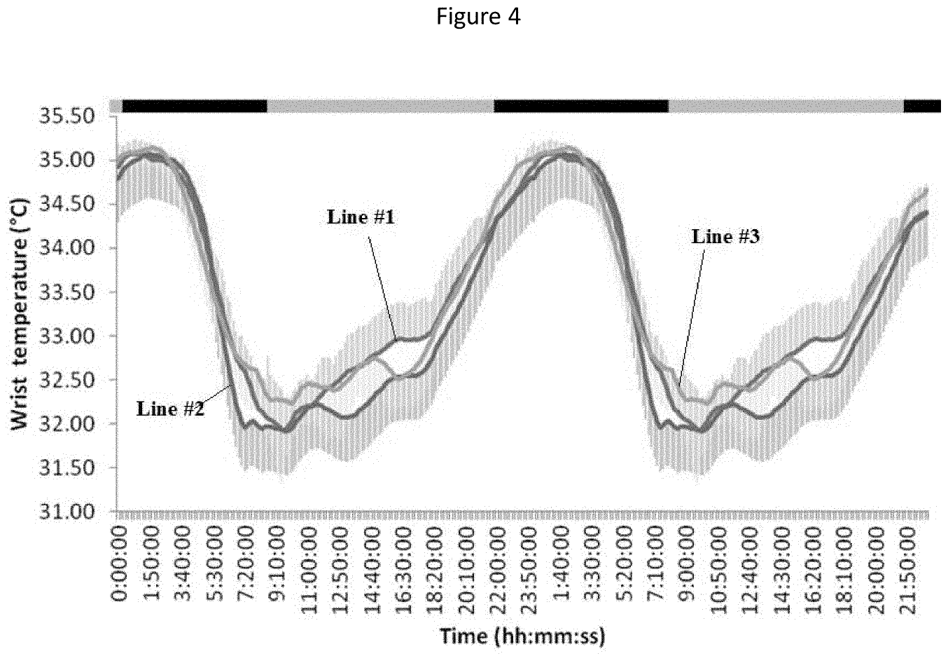

Example 1 describes calibration of a fixed aminosterol dose for a specific PD patient using constipation as the symptom or marker by which improvement was measured. In Example 1, the degree of constipation was measured by the number of complete spontaneous bowel movement (CSBM) or spontaneous bowel movement (SBM) per week, with an increase in the number of CSBM or SBM per week demonstrating a desired escalated aminosterol dose. Data detailed in Example 1 shows that 80% of subjects responded to aminosterol treatment with improved bowel function (see FIG. 4A), with the cumulative response rate increasing in a dose-dependent fashion from 25% at 25 mg to a maximum of 80% at 200 mg (Stage 1, FIG. 4A). In Stage 2 of the study, the response rate increased in a dose-dependent fashion from 26% at 75 mg to 85.3% at 250 mg (FIG. 4A). The dose required for a bowel response was patient-specific and varied from 75 mg to 250 mg. The median efficacious dose was 100 mg. The dose that proved efficacious in inducing a bowel response was strongly related to constipation severity at baseline (FIG. 4B); patients with baseline constipation of <1 CSBM/week required higher doses for a response (mean 192 mg) than patients with ≥1 CSBM/week (mean 120 mg). Thus, the severity of constipation correlates with a higher required “fixed escalated aminosterol dose.”

The observation that the aminosterol dose required to achieve a desired response increases with symptom severity supports the hypothesis that the greater the burden of αS impeding neuronal function, the higher the dose of aminosterol required to restore normal function and improve or resolve the symptom. It is theorized that the aminosterol dose required to obtain a positive effect in a subject for the symptom being evaluated correlates with the extent of neuronal damage. Thus, it is theorized that greater neuronal damage correlates with a higher required aminosterol dose to obtain a positive effect in a subject for the symptom being evaluated. For example, the symptom to be evaluated may be any one of the symptoms detailed herein for depression, and the clinical tools or scales described herein may be used for measuring improvement in depression symptoms to calibrate the aminosterol dosage for a particular patient.

In calibrating the fixed aminosterol dose for a specific patient, the starting dose is varied based upon the severity of the depression. Thus, for subjects with severe depression based on a baseline score on a clinical scale or tool that correlates with severe depression, oral aminosterol dosing is started at dose is from about 75 to about 175 mg/day mg or more (or any amount in-between these values as described herein). For subjects with mild or moderate depression based on a baseline score on a clinical scale or tool that correlates with mild or moderate depression, oral aminosterol dosing is started at about 1 to about 75 mg/day (or any amount in-between these values as described herein). For subjects with severe depression based on a baseline score on a clinical scale or tool that correlates with severe depression, nasal aminosterol dosing is started at dose is from about 3 to about 6 mg/day mg or more (or any amount in-between these values as described herein). For subjects with mild or moderate depression based on a baseline score on a clinical scale or tool that correlates with mild or moderate depression, nasal aminosterol dosing is started at about 0.001 to about 3 mg/day (or any amount in-between these values as described herein). Dosing for both patients is then escalated by defined amounts over a defined period of time until the fixed escalated dose for the patient is identified.

ii. Alzheimer's Disease (AD), MSA, and Schizophrenia

Other conditions or disorders exhibiting depression, and correlated with abnormal α-synuclein (αS) pathology, and/or dysfunctional DA neurotransmission, also known as dopaminergic dysfunction, are described above in Section I B. and include, for example, AD, MSA, and Schizophrenia.

There are currently a variety of art-accepted methods for diagnosing probable AD. Typically, these methods are used in combination. These methods include determining an individual's ability to carry out daily activities and identifying changes in behavior and personality. Dementia of the AD type is followed by the manifestation of major depressive disorder in 22% of AD patients whom had never experienced major depressive disorder prior to the manifestation of dementia in AD (Rosen et al. 1991). Depression may be determined by art-accepted methods, including, but not limited to, validated instruments that assess depression (e.g., Patient Health Questionnaire-9 (PHQ-9); the Beck Depression Inventory (BDI); Zung Self-Rating Depression Scale; Center for Epidemiologic Studies-Depression Scale (CES-D); and the Hamilton Rating Scale for Depression (HRSD)).

The criteria for ‘probable Alzheimer's disease’ are described a National Institute of Aging-Alzheimer's Association workgroup (McKhann et al. 2011). According to this workgroup, for people who first exhibit the core clinical characteristics of AD dementia, evidence of biomarkers associated with the disease may enhance the certainty of the diagnosis.

Multiple system atrophy (MSA) is a progressive neurodegenerative disorder characterized by a combination of symptoms that affect both the autonomic nervous system and movement. This is caused by progressive degeneration of neurons in several parts of the brain including the substantia nigra, striatum, inferior olivary nucleus, and cerebellum. αS aggregates are formed in multiple system atrophy (MSA). MSA patients are also known to display depression at high rates, for example, one study determined that 62% of subjects had at least mild depression (Zhang et al. 2018). There is no known cure for MSA and management is primarily supportive.

Schizophrenia is a chronic progressive disorder that has at its origin structural brain changes in both white and gray matter. It is likely that these changes begin prior to the onset of clinical symptoms in cortical regions, particularly those concerned with language processing. Later, they can be detected by progressive ventricular enlargement. Current magnetic resonance imaging (MRI) technology can provide a valuable tool for detecting early changes in cortical atrophy and anomalous language processing, which may be predictive of who will develop schizophrenia.

A 2013 study of schizophrenia patients documented brain changes seen in MRI scans from more than 200 patients beginning with their first episode and continuing with scans at regular intervals for up to 15 years. The scans showed that people at their first episode had less brain tissue than healthy individuals. The findings suggest that those who have schizophrenia are being affected by something before they show outward signs of the disease. Depression is recognized as a distinct syndrome within schizophrenia (Siris et al 2001).

While not wishing to be bound by theory, it is theorized that administration of a therapeutically effective fixed dose of an aminosterol composition to a schizophrenia patient may treat and/or prevent depression related symptoms associated with schizophrenia. In some embodiments, the administration may be oral—resulting in absorption in the ENS. In some embodiments, the administration may be intranasal—resulting in stimulation of neurogenesis, which has a positive impact on the loss of brain tissue characteristic of schizophrenia subjects.

iii. Other Neurodegenerative Disorders

The methods and compositions of the invention may also be useful in treating, preventing, and/or slowing the onset or progression of depression related with α-synuclein (αS) pathology, and/or dysfunctional DA neurotransmission, also known as dopaminergic dysfunction, where the underlying condition is a variety of other neurodegenerative disorders. Examples are given above in Section I.B., and include but are not limited to, Huntington's disease (HD), progressive supranuclear palsy, Frontotemporal dementia, vascular dementia, also known as multi-infarct dementia (MID) and vascular cognitive impairment (VCI), ALS, MS, SMA, and Friedreich's ataxia.

2. Psychological or Behavioral Disorders.

The methods and compositions of the invention may also be useful in treating, preventing, and/or slowing the onset or progression of depression correlated with abnormal αS pathology, and/or dysfunctional DA neurotransmission, also known as dopaminergic dysfunction, where the underlying condition is a psychological or behavioral disorder. Examples are given above in Section I.B as well as below, and include but are not limited to, agitation, anxiety, delirium, irritability, illusion and delusions, amnesia, autism, apathy, bipolar disorder, disinhibition, aberrant motor and obsessive-compulsive behaviors, or sleep disorders.

i. Sleep Disorders

Normal sleep is critically important for the proper functioning of many organ systems, the most important of which is the brain. As noted in Section I.B., sleep disorders or sleep disturbances are correlated with depression.

Disturbances in normal sleep patterns are closely associated with the normal aging process, with the development of depression, with impaired memory deposition and consolidation and with the occurrence of neurodevelopmental, neuroaffective and neurodegenerative disorders. The alternating pattern of sleep and wakefulness occurring every 24 hours is known as the circadian rhythm. The rhythm is set by the “Zeitgeber” (time setter), an entity known as the suprachiasmatic nucleus (SCN) and located in the hypothalamus. The SCN is normally “entrained” or synchronized by the external light-dark cycle. This relationship between external light and dark and the sleep wake cycle synchronized to it by the SCN can be over ridden during periods of hunger by neural signals emanating in the gut and relayed to the hypothalamus. The circadian sleep-wake cycle can also shift in response to changes in external light-dark cycles, such as the desynchronization that occurs during travel from one time zone to another (jet-lag). Under such circumstances, a progressive adjustment occurs until the SCN is resynchronized with the external light-dark cycle. A similar “phase-shift” and adjustment occurs in night-shift workers.

Under normal circumstances, the properly functioning SCN, synchronized to the external light-dark cycle and to neural signals emanating from the enteric nervous system, will regulate the sleep-wake cycle by sending neural and chemical signals to the surrounding structures and to portions of the brain stem involved in sleep and wakefulness. An individual with a properly functioning hypothalamus and brain stem will go to bed and fall asleep within minutes, remain asleep throughout the night, wake up in the morning and remain awake and alert throughout the day. During the night, the asleep individual will experience several cycles of sleep, beginning with light sleep, progressing through rapid eye movement sleep (REM-sleep) to deep sleep and back. Each complete sleep period lasts about 90 minutes. Periods of REM-sleep are closely associated with dreaming. During REM-sleep, neural signals emanating from certain parts of the brain stem ensure that skeletal muscles become “atonic” or are paralyzed, such that the individual can't “act out” their dreams.

Certain diseases and conditions are associated with abnormal functioning of the “Zeitgeber” or circadian clock, including depression (Germain et al 2008) or AD. These conditions may be reversible, such as desynchronization resulting from AD. In contrast, damage to the nerves carrying light-dark related information from the retina to the SCN (conditions which may lead to blindness), or damage to the enteric nerves and neural structures which relay messages from the intestine to the SCN (conditions which may lead to neurodegenerative disorders) can cause permanent dysfunction of the circadian rhythm and abnormal sleep behavior.

Dysfunction of the circadian rhythm manifests first and foremost by abnormal sleep patterns. Such abnormalities typically are mild at onset and worsen progressively over time. A common symptom of sleep disorder is a delay in the onset of sleep. This delay can be as long as several hours, and the individual may not be able to fall asleep until the early hours of the morning. Another common symptom is sleep fragmentation, meaning that the individual awakens several times during the course of the night. Once awakened, the individual may not be able to get back to sleep, and each awake fragment may last an hour or more, further reducing “total sleep time,” which is calculated by subtracting total time of the awake fragments from total time spent in bed. Total sleep time also diminishes with age, from about 14 to about 16 hours a day in newborns, to about 12 hours by one year of age, to about 7 to about 8 hours in young adults, progressively declining to about 5 to about 6 hours in elderly individuals. Total sleep time can be used to calculate an individual's “sleep age” and to compare it to their chronologic age. Significant discrepancies between sleep age and chronologic age are a reflection of the severity of the sleep disorder. “Sleep efficiency,” defined as the percentage of the time spent in bed asleep is another index that can be used to determine the severity of the sleep disorder. Sleep efficiency is said to be abnormal when the percentage is below about 70%.

Sleep disorders and/or sleep disturbances include but are not limited to REM-behavior disorders, disturbances in the Circadian rhythm, delayed sleep onset, sleep fragmentation, and hallucinations. Other sleep disorders or disturbances that can be treated and/or prevented according to the disclosed methods include but are not limited to hypersomnia (i.e., daytime sleepiness), parasomnias (such as nightmares, night terrors, sleepwalking, and confusional arousals), periodic limb movement disorders (such as Restless Leg Syndrome), jet lag, narcolepsy, advanced sleep phase disorder, non-24 hour sleep-wake syndrome.

Individuals with severe sleep disorders also typically suffer from day-time sleepiness. This can manifest as day-time “napping” for an hour or two, to “dosing off” for a few minutes during a film or to “micro-sleep” episodes lasting seconds to minutes, and of which the individual may or may not be aware. Narcolepsy is a rare and extreme form of day-time sleepiness, with the sudden onset of sleep causing the individual to fall down. Another form of sleep disturbance involves periods of loud snoring alternating with periods of “sleep apnea” (arrested breathing), a condition known as “sleep-disordered breathing.” “REM-behavior disorder” (RBD) or “REM-disturbed sleep”, is yet another sleep disturbance which occurs as a result of dysfunctional neural communication between the enteric nervous system, structures responsible for sleep in the brain stem and the SCN. In individuals with RBD, neural signaling which causes the paralysis (atonia) of muscles under voluntary control is impaired or altogether absent. As a consequence, “acting-out” of dreams occurs. This can range at one end of the spectrum from an increase in muscle tone detectable by electromyography (EMG) and accompanied by small movements of the hands and feet during REM sleep, to violent thrashing of arms and legs, kicking or punching a bed partner, speaking out loud or screaming, at the other end of the spectrum. Episodes of RBD can occur several times a night or very infrequently, once every few months. They can also be clustered, several occurring within a week, followed by periods of normal sleep. Unless the condition can be treated with a medication that restores normal functioning of the circadian rhythm and improves sleep patterns, individuals with RBD progress to neurodegenerative disorders.

Sleep disturbances include but are not limited to RBD, circadian rhythm dysfunction, delayed sleep onset, Restless leg syndrome, daytime sleepiness, and sleep fragmentation.

Sleep is increasingly recognized as important to public health, with sleep insufficiency linked to motor vehicle crashes, industrial disasters, and medical and other occupational errors. Unintentionally falling asleep, nodding off while driving, and having difficulty performing daily tasks because of sleepiness all may contribute to these hazardous outcomes. Persons experiencing sleep insufficiency are also more likely to suffer from chronic diseases such as hypertension, diabetes, depression, and obesity, as well as from cancer, increased mortality, and reduced quality of life and productivity. Sleep insufficiency may be caused by broad scale societal factors such as round-the-clock access to technology and work schedules, but sleep disorders such as insomnia or obstructive sleep apnea also play an important role. An estimated 50-70 million US adults have a sleep or wakefulness disorder.

A “normal” or “restful” sleep period is defined as a sleep period uninterrupted by wakefulness. Alternatively, a said period can be defined by the recommended or appropriate amount of sleep for the subject's age category, e.g., (i) infants 0-3 months=about 11 to about 19 hours; (ii) infants about 4 to about 11 months=about 12 to about 18 hours; (iii) toddlers about 1 to about 2 years=about 9 to about 16 hours; (iv) preschoolers about 3 to about 5 years=about 10 to about 14 hours; (v) school-aged children about 6 to about 13 years=about 7 to about 12 hours; (v) teenagers about 14 to about 17 years=about 7 to about 11 hours; (vi) young adults about 18 to about 25 years=about 6 to about 11 hours; (vii) adults about 26 to about 64 years=about 6 to about 10 hours; and (viii) older adults >65 years=about 5 to about 9 hours. Thus, for treating sleep disturbance in a subject, the treatment can result in a restful sleep period of at least about 4, about 5, about 6, about 7, about 8, about 9, about 10, about 11, or about 12 hours.

How much sleep is needed by a subject varies between individuals but generally changes with age. The National Institutes of Health suggests that school-age children need at least 10 hours of sleep daily, teens need 9-10 hours, and adults need 7-8 hours. According to data from the National Health Interview Survey, nearly 30% of adults reported an average of ≤6 hours of sleep per day in 2005-2007. Further, in 2009, only 31% of high school students reported getting at least 8 hours of sleep on an average school night. Similar recommendations are provided by the National Sleep Foundation (https://sleepfoundation.org/press-release/national-sleep-foundation-recommends-new-sleep-times/page/0/1):

TABLE 1

| | | May be | |
|---|---|---|---|
| Age | Recommended | appropriate | Not recommended |
| Newborns | 14 to 17 hours | 11 to 13 hours | Less than 11 hours |
| 0-3 months | | 18 to 19 hours | More than 19 hours |
| Infants | 12 to 15 hours | 10 to 11 hours | Less than 10 hours |
| 4-11 months | | 16 to 18 hours | More than 18 hours |
| Toddlers | 11 to 14 hours | 9 to 10 hours | Less than 9 hours |
| 1-2 years | | 15 to 16 hours | More than 16 hours |
| Preschoolers | 10 to 13 hours | 8 to 9 hours | Less than 8 hours |
| 3-5 years | | 14 hours | More than 14 hours |
| School-aged | 9 to 11 hours | 7 to 8 hours | Less than 7 hours |
| Children | | 12 hours | More than 12 hours |
| 6-13 years | | | |
| Teenagers | 8 to 10 hours | 7 hours | Less than 7 hours |
| 14-17 years | | 11 hours | More than 11 hours |
| Young Adults | 7 to 9 hours | 6 hours | Less than 6 hours |
| 18-25 years | | 10 to 11 hours | More than 11 hours |
| Adults | 7 to 9 hours | 6 hours | Less than 6 hours |
| 26-64 years | | 10 hours | More than 10 hours |
| Older Adults | 7 to 8 hours | 5 to 6 hours | Less than 5 hours |
| ≥65 years | | 9 hours | More than 9 hours |

There are several different scientifically acceptable ways to measure a sleep period uninterrupted by wakefulness. First, electrodes attached to the head of a subject can measure electrical activity in the brain by electroencephalography (EEG). This measure is used because the EEG signals associated with being awake are different from those found during sleep. Second, muscle activity can be measured using electromyography (EMG), because muscle tone also differs between wakefulness and sleep. Third, eye movements during sleep can be measured using electro-oculography (EOG). This is a very specific measurement that helps to identify Rapid Eye Movement or REM sleep. Any of these methods, or a combination thereof, can be used to determine if a subject obtains a restful sleep period following administration of at least one aminosterol or a salt or derivative thereof to the subject.

Further, circadian rhythm regulation can be monitored in a variety of ways, including but not limited to monitoring wrist skin temperature as described by Sarabia et al. 2008. Similarly symptoms of RBD can be monitored using a daily diary and RBD questionnaire (Stiasny-Kolster et al. 2007).

In some embodiments, administration of a therapeutically effective fixed dose of an aminosterol composition to a depression patient with disturbed sleep results in improvement in frequency of normal or restful sleep as determined by a clinically recognized assessment scale for one or more types of sleep dysregulation, by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%. The improvement can be measured using any clinically recognized tool or assessment.

Example 1 describes several tools used to measure and evaluate the effect of aminosterol treatment on sleep, including for example:

(1) Sleep Diary (participants completed a sleep diary on a daily basis throughout the study. The diaries included time into bed and estimated time to sleep as well as wake time and duration during the night.);

(2) I-Button Temperature Assessment. The I-Button is a small, rugged self-sufficient system that measures temperature and records the results in a protected memory section. The Thermochron I-Button DS1921H (Maxim Integrated, Dallas, Tex.) was used for skin temperature measurement. I-Buttons were programmed to sample every 10 mins., and attached to a double-sided cotton sport wrist band using Velcro, with the sensor face of the I-Button placed over the inside of the wrist, on the radial artery of the dominant hand. Subjects removed and replaced the data logger when necessary (i.e., to have a bath or shower). The value of skin temperature assessment in sleep research is that the endogenous skin warming resulting from increased skin blood flow is functionally linked to sleep propensity. From the collected data, the mesor, amplitude, acrophase (time of peak temperature), Rayleight test (an index of interdaily stability), mean waveforms are calculated);

(3) Unified Parkinson's Disease Rating Scale (UPDRS), sections 1.7 (sleep problems), 1.8 (daytime sleepiness) and 1.13 (fatigue);

(4) Parkinson's Disease Fatigue Scale (PFS-16);

(5) REM Sleep Behavior Disorder Screening Questionnaire; and

(6) Parkinson's Disease Sleep Scale.

The data detailed in Example 1 described how circadian system status was evaluated by continuously monitoring wrist skin temperature (Thermochron iButton DS1921H; Maxim, Dallas) following published procedures (Sarabia et al. 2008). Further, an analysis was done with respect to the sleep data, the body temperature data, and fatigue data. The frequency of arm or leg thrashing reported in the sleep diary diminished progressively from 2.2 episodes/week at baseline to 0 at maximal dose (100% improvement). Total sleep time increased progressively from 7.1 hours at baseline to 8.4 hours at 250 mg (an 18% increase) and was consistently higher than baseline beyond 125 mg (FIG. 4). Unlike stool-related indices, the improvement in many CNS symptoms persisted during wash-out.

Circadian rhythm of skin temperature was evaluable in 12 patients (i.e., those who had recordings that extended from baseline through washout). Circadian system functionality was evaluated by continuously monitoring wrist skin temperature using a temperature sensor (Thermochron iButton DS1921H; Maxim, Dallas, Tex.) (Sarabia et al. 2008). Briefly, this analysis includes the following parameters: (i) the inter-daily stability (the constancy of 24-hour rhythmic pattern over days, IS); (ii) intra-daily variability (rhythm fragmentation, IV); (iii) average of 10-minute intervals for the 10 hours with the minimum temperature (L10); (iv) average of 10-minute intervals for the 5 hours with the maximum temperature (M5) and the relative amplitude (RA), which was determined by the difference between M5 and L10, divided by the sum of both. Finally, the Circadian Function Index (CFI) was calculated by integrating IS, IV, and RA. Consequently, CFI is a global measure that oscillates between 0 for the absence of circadian rhythmicity and 1 for a robust circadian rhythm.

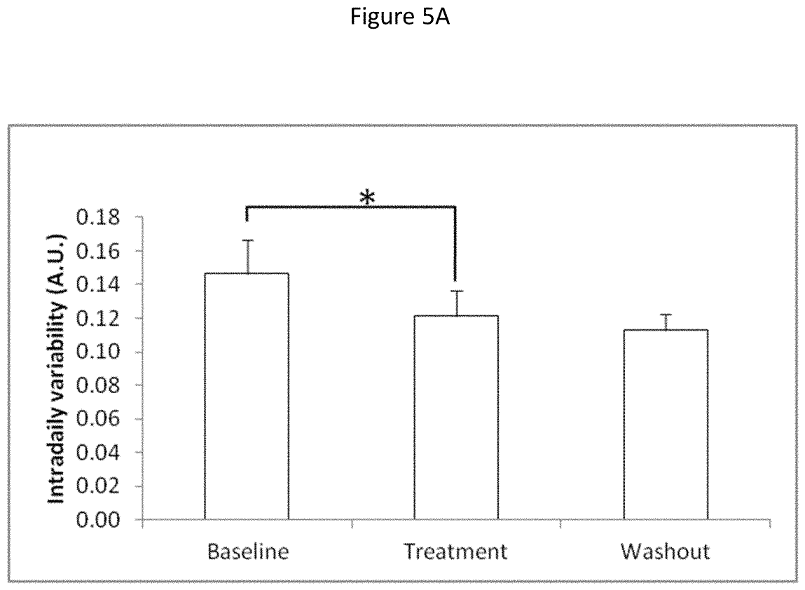

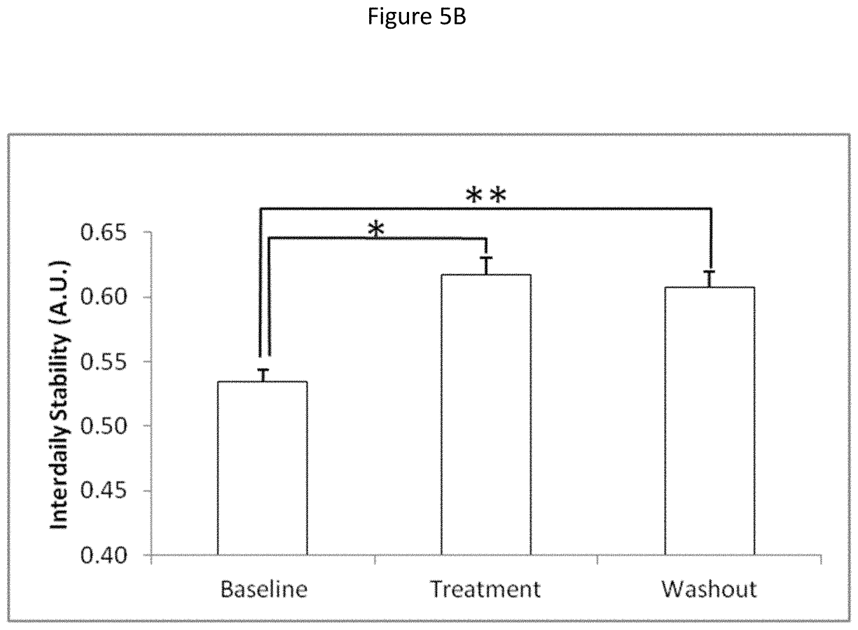

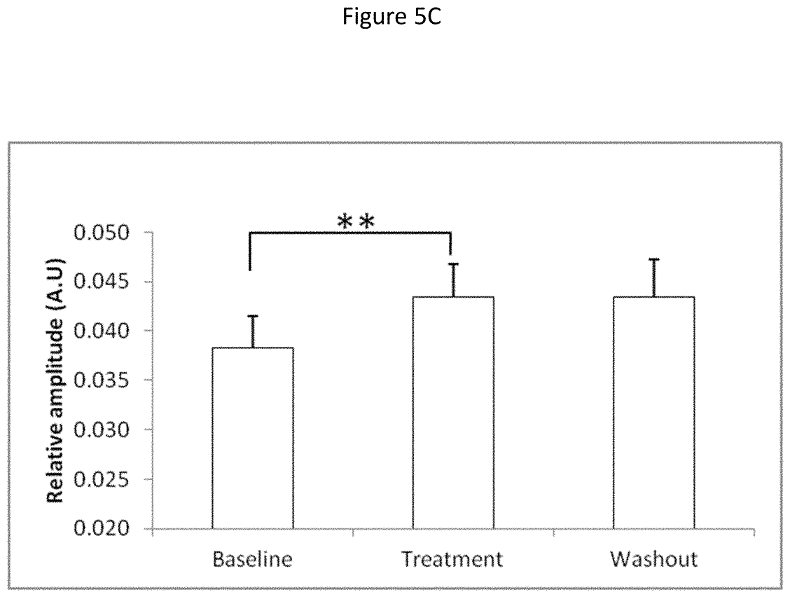

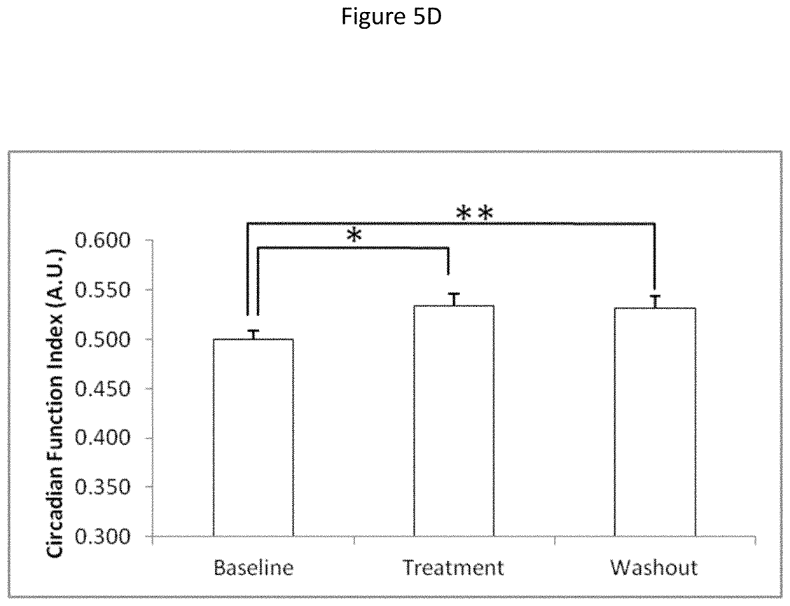

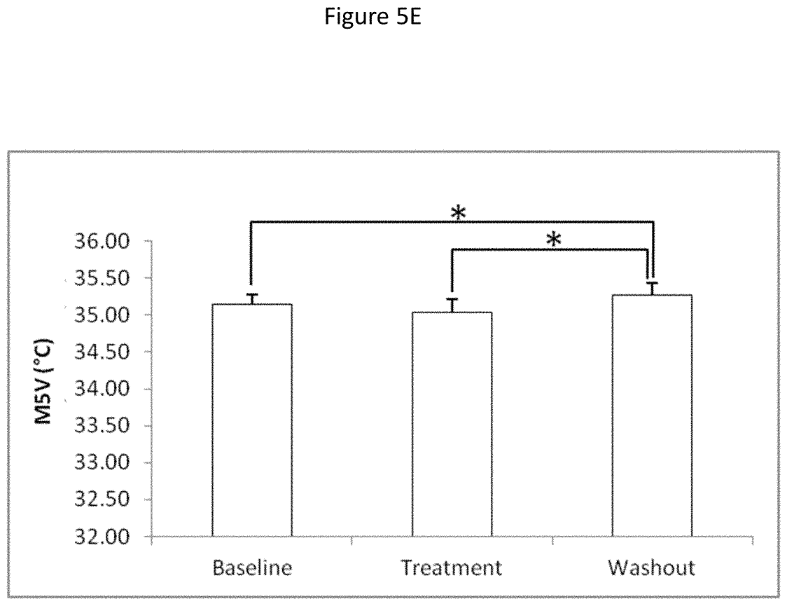

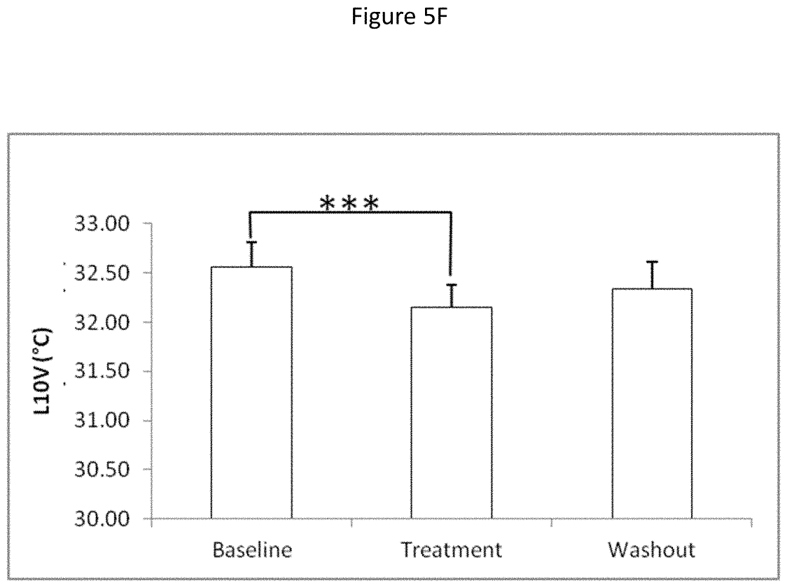

A comparison was performed of circadian rhythm parameters during the baseline, fixed dose and washout periods. Aminosterol administration improved all markers of healthy circadian function, including increasing rhythm stability, relative amplitude, and circadian function index, while reducing rhythm fragmentation. The improvement persisted for several of these circadian parameters during the wash-out period. (FIG. 5). Improvements were also seen in REM-behavior disorder (RBD) and sleep. RBD and total sleep time also improved progressively in a dose-dependent manner.

ii. Cognitive Impairment

Cognitive impairment, including mild cognitive impairment (MCI), is characterized by increased memory or thinking problems exhibited by a subject as compared to a normal subject of the same age. Approximately 15 to 20 percent of people age 65 or older have MCI, and MCI is especially linked to depression (Dfrancesco et al., 2018) or synucleopathies like Parkinson's disease (PD), which also is associated with depression as discussed above. In 2002, an estimated 5.4 million people (22%) in the United States over age 70 had cognitive impairment without dementia (Plassman et al. 2009).

Cognitive impairment may entail memory problems including a slight but noticeable and measurable decline in cognitive abilities, including memory and thinking skills. When MCI primarily affects memory, it is known as “amnestic MCI.” A person with amnestic MCI may forget information that would previously have been easily recalled, such as appointments, conversations, or recent events, for example. When MCI primarily affects thinking skills other than memory, it is known as “nonamnestic MCI.” A person with nonamnestic MCI may have a reduced ability to make sound decisions, judge the time or sequence of steps needed to complete a complex task, or with visual perception, for example.

Mild cognitive impairment is a clinical diagnosis. A combination of cognitive testing and information from a person in frequent contact with the subject is used to fully assess cognitive impairment. A medical workup includes one or more of an assessment by a physician of a subject's medical history (including current symptoms, previous illnesses, and family history), assessment of independent function and daily activities, assessment of mental status using brief tests to evaluate memory, planning, judgment, ability to understand visual information, and other key thinking skills, neurological examination to assess nerve and reflex function, movement, coordination, balance, and senses, evaluation of mood, brain imaging, or neuropsychological testing. Diagnostic guidelines for MCI have been developed by various groups, including the Alzheimer's Association partnered with the National Institute on Aging (NIA), an agency of the U.S. National Institutes of Health (NIH). Jack et al. 2011; McKhann et al. 2011; Albert et al. 2011. Recommendations for screening for cognitive impairment have been issued by the U.S. Preventive Services Task Force. Screening for Cognitive Impairment in Older Adults, U.S. Preventive Services Task Force (March 2014), https://www.uspreventiveservicestaskforce.org/Home/GetFileByID/1882. For example, the Mini Mental State Examination (MMSE) may be used. Palsetia et al. (2018); Kirkevold, O. & Selbaek, G. (2015). With the MMSE, a score of 24 or greater (out of 30) may indicate normal cognition, with lower scores indicating severe (less than or equal to 9 points), moderate (10-18 points), or mild (19-23 points) cognitive impairment. Other screening tools include the Informant Questionnaire on Cognitive Decline in the Elderly (IQCODE), in which an average score of 3 indicates no cognitive decline and a score greater than 3 indicates some decline. Jorm, A. F. 2004. Alternatively, the 7-Minute Screener, Abbreviated Mental Test Score (AMTS), Cambridge Cognitive Examination (CAMCOG), Clock Drawing Test (CDT), General Practitioner Assessment of Cognition (GPCOG), Mini-Cog, Memory Impairment Screen (MIS), Montreal Cognitive Assessment (MoCA), Rowland Universal Dementia Assessment (RUDA), Self-Administered Gerocognitive Examination (SAGE), Short and Sweet Screening Instrument (SAS-SI), Short Blessed Test (SBT), St. Louis Mental Status (SLUMS), Short Portable Mental Status Questionnaire (SPMSQ), Short Test of Mental Status (STMS), or Time and Change Test (T&C), among others, are frequently employed in clinical and research settings. Cordell et al. 2013. Numerous examinations may be used, as no single tool is recognized as the “gold standard,” and improvements in score on any standardized examination indicate successful treatment of cognitive impairment, whereas obtaining a score comparable to the non-impaired population indicates total recovery.

In some embodiments, administration of a therapeutically effective fixed dose of an aminosterol composition to a depression patient in need results in improvement of cognitive impairment as determined by a clinically recognized assessment scale, by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%. The improvement can be measured using any clinically recognized tool or assessment.

As detailed in Example 1, cognitive impairment and the improvement following aminosterol treatment were assessed using several tools:

(1) Mini Mental State Examination (MMSE);

(2) Trail Making Test (TMT) Parts A and B; and

(3) Unified Parkinson's Disease Rating Scale (UPDRS), sections 1.1 (cognitive impairment).

Assessments were made at baseline and at the end of the fixed dose and washout periods for Example 1, and an analysis was done with respect to the cognition symptoms. The results showed that the total UPDRS score was 64.4 at baseline, 60.6 at the end of the fixed dose period and 55.7 at the end of the wash-out period (a 13.5% improvement). Part 1 of the UPDRS (which includes section 1.1, cognitive impairment) had a mean baseline score of 11.6, a fixed aminosterol dose mean score of 10.6, and a wash-out mean score of 9.5, demonstrating an almost 20% improvement (UPDRS cognitive impairment is rated from 1=slight improvement to 4=severe impairment, so lower scores correlate with better cognitive function). In addition, MMSE improved from 28.4 at baseline to 28.7 during treatment and to 29.3 during wash-out (the MMSE has a total possible score of 30, with higher scores correlating with better cognitive function). Unlike stool-related indices, the improvement in many CNS symptoms persisted during wash-out.

3. Cerebral and General Ischemic Disorders

There is abundant and increasing evidence from these different lines of research that depression has a bidirectional association with vascular diseases (Thomas et al. 2004). α-S is normally expressed in vascular cells and may play some physiological role in the vascular wall (Tamo et al. 2002), thus α-S dysfunction. The methods and compositions of the invention may also be useful in treating, preventing, and/or delaying the onset or progression of depression and/or a depression-related symptom, where the depression is correlated with abnormal α-S pathology, and/or correlated with dysfunctional DA neurotransmission, also known as dopaminergic dysfunction, and wherein the depression is also correlated with a cerebral or general ischemic disorder.

In some embodiments, the cerebral ischemic disorder comprises cerebral microangiopathy, intrapartal cerebral ischemia, cerebral ischemia during/after cardiac arrest or resuscitation, cerebral ischemia due to intraoperative problems, cerebral ischemia during carotid surgery, chronic cerebral ischemia due to stenosis of blood-supplying arteries to the brain, sinus thrombosis or thrombosis of cerebral veins, cerebral vessel malformations, or diabetic retinopathy.

In some embodiments, the general ischemic disorders comprises high blood pressure, high cholesterol, myocardial infarction, cardiac insufficiency, cardiac failure, congestive heart failure, myocarditis, pericarditis, perimyocarditis, coronary heart disease, angina pectoris, congenital heart disease, shock, ischemia of extremities, stenosis of renal arteries, diabetic retinopathy, thrombosis associated with malaria, artificial heart valves, anemias, hypersplenic syndrome, emphysema, lung fibrosis, or pulmonary edema.

V. Combination Therapy.

In the methods of the invention, the aminosterol compositions may be administered alone or in combination with other therapeutic agents. An example of an additional therapeutic agent is one known to be useful in treating depression and/or a depression related disease or disorder.

Thus, any active agent known to be useful in treating a condition, disease, or disorder described herein can be used in the methods of the invention, and either combined with the aminosterol compositions used in the methods of the invention, or administered separately or sequentially.

For example, in methods of treating, preventing, and/or delaying the onset or progression of depression or related symptoms associated with PD, the aminosterol composition can be co-administered or combined with drugs commonly prescribed to treat PD or related symptoms, such as levodopa (usually combined with a dopa decarboxylase inhibitor or COMT inhibitor), dopamine agonists and MAO-B inhibitors. Exemplary dopa decarboxylase inhibitors are carbidopa and benserazide. Exemplary COMT inhibitors are tolcapone and entacapone. Dopamine agonists include, for example, bromocriptine, pergolide, pramipexole, ropinirole, piribedil, cabergoline, apomorphine, lisuride, and rotigotine. MAO-B inhibitors include, for example, selegiline and rasagiline. Other drugs commonly used to treat PD include, for example, amantadine, anticholinergics, clozapine for psychosis, cholinesterase inhibitors for dementia, and modafinil for daytime sleepiness.

In methods of treating, preventing, and/or delaying the onset or progression of depression or related symptoms associated with AD, the aminosterol composition can be co-administered or combined with drugs commonly prescribed to treat AD or related symptoms, such as glutamate, antipsychotic drugs, huperzine A, acetylcholinesterase inhibitors and NMDA receptor antagonists such as memantine (Akatinol®, Axura®, Ebixa®/Abixa®, Memox® and Namenda®). Examples of acetylcholinesterase inhibitors are donepezil (Aricept®), galantamine (Razadyne®), and rivastigmine (Exelon®).

In methods of treating, preventing, and/or delaying the onset or progression of depression or related symptoms associated with diabetes and/or diabetes mellitus, including both Type 1 and Type 2 diabetes, or neuropathy of diabetes, the aminosterol composition can be co-administered or combined with drugs commonly prescribed to treat diabetes mellitus or related symptoms, such as insulin (NPH insulin or synthetic insulin analogs) (e.g., Humulin®, Novolin®) and oral antihyperglycemic drugs. Oral antihyperglycemic drugs include but are not limited to (1) biguanides such as metformin (Glucophage®); (2) Sulfonylureas such as acetohexamide, chlorpropamide (Diabinese®), glimepiride (Amaryl®), Glipizide (Glucotrol®), Tolazamide, Tolbutamide, and glyburide (Diabeta®, Micronase®); (3) Meglitinides such as repaglinide (Prandin®) and nateglinide (Starlix®); (4) Thiazolidinediones such as rosiglitazone (Avandia®) and pioglitazone (Actos®); (5) Alpha-glucosidase inhibitors such as acarbose (Precose®) and miglitol (Glyset®); (6) Dipeptidyl peptidase-4 inhibitors such as Sitagliptin (Januvia®); (7) Glucagon-like peptide agonists such as exenatide (Byetta®); and (8) Amylin analogs such as pramlintide (Symlin®).

In methods of treating, preventing, and/or delaying the onset or progression of depression or related symptoms associated with Huntington's chorea or disease, the aminosterol composition can be co-administered or combined with drugs commonly prescribed to treat Huntington's chorea or related symptoms, such as medications prescribed to help control emotional and movement problems associated with Huntington's chorea. Such medications include, but are not limited to, (1) antipsychotic drugs, such as haloperidol and clonazepam; (2) drugs used to treat dystonia, such as acetylcholine regulating drugs (trihexyphenidyl, benztropine (Cogentin®), and procyclidine HCl); GABA-regulating drugs (diazepam (Valium®), lorazepam (Ativan®), clonazepam (Klonopin®), and baclofen (Lioresal®)); dopamine-regulators (levodopa/carbidopa (Sinemet®), bromocriptine (parlodel), reserpine, tetrabenazine); anticonvulsants (carbamazepine (Tegretol®) and botulinum toxin (Botox®)); and (3) drugs used to treat depression (fluoxetine, sertraline, and nortriptyline). Other drugs commonly used to treat HD include amantadine, tetrabenazine, dopamine blockers, and co-enzyme Q₁₀.

In methods of treating, preventing, and/or delaying the onset or progression of depression or related symptoms associated with peripheral sensory neuropathy, the aminosterol composition can be co-administered or combined with drugs commonly prescribed to treat peripheral sensory neuropathy or related symptoms. Peripheral sensory neuropathy refers to damage to nerves of the peripheral nervous system, which may be caused either by diseases of or trauma to the nerve or the side-effects of systemic illness. Drugs commonly used to treat this condition include, but are not limited to, neurotrophin-3, tricyclic antidepressants (e.g., amitriptyline), antiepileptic therapies (e.g., gabapentin or sodium valproate), synthetic cannabinoids (Nabilone) and inhaled cannabis, opioid or opiate derivatives, and pregabalin (Lyrica®).

In methods of treating, preventing, and/or delaying the onset or progression of depression or related symptoms associated with traumatic head and/or spine injury, the aminosterol composition can be co-administered or combined with drugs commonly prescribed to treat traumatic head and/or spine injury or related symptoms, such as analgesics (acetaminophen, NSAIDs, salicylates, and opioid drugs such as morphine and opium) and paralytics.

In methods of treating, preventing, and/or delaying the onset or progression of depression or related symptoms associated with stroke, the aminosterol composition can be co-administered or combined with drugs commonly prescribed to treat stroke or related symptoms, such as aspirin, clopidogrel, dipyridamole, tissue plasminogen activator (tPA), and anticoagulants (e.g., alteplase, warfarin, dabigatran).

In methods of treating, preventing, and/or delaying the onset or progression of depression or related symptoms associated with ALS, the aminosterol composition can be co-administered or combined with drugs commonly prescribed to treat Amyotrophic lateral sclerosis or related symptoms, such as riluzole (Rilutek®), KNS-760704 (an enantiomer of pramipexole), olesoxime (TRO19622), talampanel, arimoclomol, medications to help reduce fatigue, ease muscle cramps, control spasticity, reduce excess saliva and phlegm, control pain, depression, sleep disturbances, dysphagia, and constipation.

In methods of treating, preventing, and/or delaying the onset or progression of depression or related symptoms associated with multiple sclerosis, the aminosterol composition can be co-administered or combined with drugs commonly prescribed to treat multiple sclerosis or related symptoms, such as corticosteroids (e.g., methylprednisolone), plasmapheresis, fingolimod (Gilenya®), interferon beta-la (Avonex®, CinnoVex®, ReciGen® and Rebift), interferon beta-lb (Betaseron® and Betaferon®), glatiramer acetate (Copaxone®), mitoxantrone, natalizumab (Tysabri®), alemtuzumab (Campath®), daclizumab (Zenapax®), rituximab, dirucotide, BHT-3009, cladribine, dimethyl fumarate, estriol, fingolimod, laquinimod, minocycline, statins, temsirolimus teriflunomide, naltrexone, and vitamin D analogs.

In methods of treating, preventing, and/or delaying the onset or progression of depression or related symptoms associated with cerebral palsy, the aminosterol composition can be co-administered or combined with drugs commonly prescribed to treat cerebral palsy or related symptoms, such as botulinum toxin A injections.

In methods of treating, preventing, and/or delaying the onset or progression of depression or related symptoms associated with epilepsy, the aminosterol composition can be co-administered or combined with drugs commonly prescribed to treat epilepsy or related symptoms, such as anticonvulsants (e.g., carbamazepine (Tegretol®), clorazepate (Tranxene®), clonazepam (Klonopin®), ethosuximide (Zarontin®), felbamate (Felbatol®), fosphenytoin (Cerebyx®), gabapentin (Neurontin®), lacosamide (Vimpat®), lamotrigine (Lamictal®), levetiracetam (Keppra®), oxcarbazepine (Trileptal®), phenobarbital (Luminal®), phenytoin (Dilantin®), pregabalin (Lyrica®), primidone (Mysoline®), tiagabine (Gabitril®), topiramate (Topamax®), valproate semisodium (Depakote®), valproic acid (Depakene®), and zonisamide (Zonegran®), clobazam (Frisium®), vigabatrin (Sabril®), retigabine, brivaracetam, seletracetam, diazepam (Valium® and Diastat®), lorazepam (Ativan®), paraldehyde (Paral®), midazolam (Versed®), pentobarbital (Nembutal®), acetazolamide (Diamox®), progesterone, adrenocorticotropic hormone (ACTH and Acthar®), various corticotropic steroid hormones (prednisone), and bromide.

In methods of treating, preventing, and/or delaying the onset or progression of depression or related symptoms associated with cognitive impairment, the aminosterol composition can be co-administered or combined with drugs commonly prescribed to treat cognitive impairment, such as donepezil (Aricept®), galantamine (Razadyne®), rivastigmine (Exelon®), and stimulants such as caffeine, amphetamine (Adderall®), lisdexamfetamine (Vyvanse®), and methylphenidate (Ritalin®); NMDA antagonists such as memantine (Nameda®) and ketamine; supplements such as ginko biloba, L-theanine, piracetam, oxiracitam, aniracetam, tolcapone, atomoxetine, ginseng, and salvia officinalis.

In the methods of treating, preventing, and/or delaying the onset or progression of depression or related symptoms associated with malignancies, the aminosterol composition can be co-administered or combined with drugs commonly used to treat malignancies. These include all known cancer drugs, such as but not limited to those listed at http://www.cancer.gov/cancertopics/druginfo/alphalist as of May 5, 2014, which is specifically incorporated by reference. In one embodiment, the drug commonly used to treat malignancies may be selected from the group consisting of actinomycin-D, alkeran, ara-C, anastrozole, BiCNU, bicalutamide, bleomycin, busulfan, capecitabine, carboplatin, carboplatinum, carmustine, CCNU, chlorambucil, cisplatin, cladribine, CPT-11, cyclophosphamide, cytarabine, cytosine arabinoside, cytoxan, dacarbazine, dactinomycin, daunorubicin, dexrazoxane, docetaxel, doxorubicin, DTIC, epirubicin, ethyleneimine, etoposide, floxuridine, fludarabine, fluorouracil, flutamide, fotemustine, gemcitabine, hexamethylamine, hydroxyurea, idarubicin, ifosfamide, irinotecan, lomustine, mechlorethamine, melphalan, mercaptopurine, methotrexate, mitomycin, mitotane, mitoxantrone, oxaliplatin, paclitaxel, pamidronate, pentostatin, plicamycin, procarbazine, steroids, streptozocin, STI-571, tamoxifen, temozolomide, teniposide, tetrazine, thioguanine, thiotepa, tomudex, topotecan, treosulphan, trimetrexate, vinblastine, vincristine, vindesine, vinorelbine, VP-16, xeloda, asparaginase, AIN-457, bapineuzumab, belimumab, brentuximab, briakinumab, canakinumab, cetuximab, dalotuzumab, denosumab, epratuzumab, estafenatox, farletuzumab, figitumumab, galiximab, gemtuzumab, girentuximab (WX-G250), herceptin, ibritumomab, inotuzumab, ipilimumab, mepolizumab, muromonab-CD3, naptumomab, necitumumab, nimotuzumab, ocrelizumab, ofatumumab, otelixizumab, ozogamicin, pagibaximab, panitumumab, pertuzumab, ramucirumab, reslizumab, rituximab, REGN88, solanezumab, tanezumab, teplizumab, tiuxetan, tositumomab, trastuzumab, tremelimumab, vedolizumab, zalutumumab, zanolimumab, 5FC, accutane hoffmann-la roche, AEE788 novartis, AMG-102, anti neoplaston, AQ4N (Banoxantrone), AVANDIA (Rosiglitazone Maleate), avastin (Bevacizumab) genetech, BCNU, biCNU carmustine, CCI-779, CCNU, CCNU lomustine, celecoxib (Systemic), chloroquine, cilengitide (EMD 121974), CPT-11 (CAMPTOSAR, Irinotecan), dasatinib (BMS-354825, Sprycel), dendritic cell therapy, etoposide (Eposin, Etopophos, Vepesid), GDC-0449, gleevec (imatinib mesylate), gliadel wafer, hydroxychloroquine, IL-13, IMC-3G3, immune therapy, iressa (ZD-1839), lapatinib (GW572016), methotrexate for cancer (Systemic), novocure, OSI-774, PCV, RAD001 novartis (mTOR inhibitor), rapamycin (Rapamune, Sirolimus), RMP-7, RTA 744, simvastatin, sirolimus, sorafenib, SU-101, SU5416 sugen, sulfasalazine (Azulfidine), sutent (Pfizer), TARCEVA (erlotinib HCl), taxol, TEMODAR schering-plough, TGF-B anti-sense, thalomid (thalidomide), topotecan (Systemic), VEGF trap, VEGF-trap, vorinostat (SAHA), XL 765, XL184, XL765, zarnestra (tipifarnib), ZOCOR (simvastatin), cyclophosphamide (Cytoxan), (Alkeran), chlorambucil (Leukeran), thiopeta (Thioplex), busulfan (Myleran), procarbazine (Matulane), dacarbazine (DTIC), altretamine (Hexalen), clorambucil, cisplatin (Platinol), ifosafamide, methotrexate (MTX), 6-thiopurines (Mercaptopurine [6-MP], Thioguanine [6-TG]), mercaptopurine (Purinethol), fludarabine phosphate, (Leustatin), flurouracil (5-FU), cytarabine (ara-C), azacitidine, vinblastine (Velban), vincristine (Oncovin), podophyllotoxins (etoposide {VP-16} and teniposide {VM-26}), camptothecins (topotecan and irinotecan), taxanes such as paclitaxel (Taxol) and docetaxel (Taxotere), (Adriamycin, Rubex, Doxil), dactinomycin (Cosmegen), plicamycin (Mithramycin), mitomycin: (Mutamycin), bleomycin (Blenoxane), estrogen and androgen inhibitors (Tamoxifen), gonadotropin-releasing hormone agonists (Leuprolide and Goserelin (Zoladex)), aromatase inhibitors (Aminoglutethimide and Anastrozole (Arimidex)), amsacrine, asparaginase (El-spar), mitoxantrone (Novantrone), mitotane (Lysodren), retinoic acid derivatives, bone marrow growth factors (sargramostim and filgrastim), amifostine, pemetrexed, decitabine, iniparib, olaparib, veliparib, everolimus, vorinostat, entinostat (SNDX-275), mocetinostat (MGCD0103), panobinostat (LBH589), romidepsin, valproic acid, flavopiridol, olomoucine, roscovitine, kenpaullone, AG-024322 (Pfizer), fascaplysin, ryuvidine, purvalanol A, NU2058, BML-259, SU 9516, PD-0332991, P276-00, geldanamycin, tanespimycin, alvespimycin, radicicol, deguelin, BIIBO21, cis-imidazoline, benzodiazepinedione, spiro-oxindoles, isoquinolinone, thiophene, 5-deazaflavin, tryptamine, aminopyridine, diaminopyrimidine, pyridoisoquinoline, pyrrolopyrazole, indolocarbazole, pyrrolopyrimidine, dianilinopyrimidine, benzamide, phthalazinone, tricyclic indole, benzimidazole, indazole, pyrrolocarbazole, isoindolinone, morpholinyl anthracycline, a maytansinoid, ducarmycin, auristatins, calicheamicins (DNA damaging agents), α-amanitin (RNA polymerase II inhibitor), centanamycin, pyrrolobenzodiazepine, streptonigtin, nitrogen mustards, nitrosorueas, alkane sulfonates, pyrimidine analogs, purine analogs, antimetabolites, folate analogs, anthracyclines, taxanes, vinca alkaloids, topoisomerase inhibitors, hormonal agents, and any combination thereof.

In the methods of treating, preventing, and/or delaying the onset or progression of depression or related symptoms, the aminosterol composition can be co-administered or combined with drugs commonly used to treat depression. These include selective serotonin reuptake inhibitors (SSRIs) such as citalopram (Celexa®, Cipramil®), escitalopram (Lexapro®, Cipralex®), paroxetine (Paxil®, Seroxat®), fluoxetine (Prozac®), fluvoxamine (Luvox®, Faverin®), sertraline (Zoloft®, Lustral®), indalpine (Upstene®), zimelidine (Normud®, Zelmid®); serotonin-norepinephrine reuptake inhibitors (SNRIs) such as desvenlafaxine (Pristiq®), duloxetine (Cymbalta®), levomilnacipran (Fetzima®), milnacipran (Ixel®, Savella®), venlafaxine (Effexor®); serotonin modulators and stimulators (SMSs) such as vilazodone (Viibryd®), vortioxetine (Trintellix®); serotonin antagonists and reuptake inhibitors such as nefazodone (Dutonin®, Nefadar®, Serzone®), trazodone (Desyrel®), etoperidone; norepinephrine reuptake inhibitors (NRIs) such as reboxetine (Edronax®), teniloxazine (Lucelan®, Metatone®), viloxazine (Vivalan®), atomoxetine (Strattera®); norepinephrine-dopamine reuptake inhibitors such as bupropion (Wellbutrin®), amineptine (Survector®, Maneon®), nomifensine (Merital®, Alival®), methylphenidate (Ritalin®, Concerta®), lisdexamfetamine (Vyvanse®); tricyclic antidepressants such asamitriptyline (Elavil®, Endep®), amitriptylinoxide (Amioxid®, Ambivalon®, Equilibrin®), clomipramine (Anafranil®), desipramine (Norpramin®, Pertofrane®), dibenzepin (Noveril®, Victoril®), dimetacrine (Istonil®), dosulepin (Prothiaden®), doxepin (Adapin®, Sinequan®), imipramine (Tofranil®), lofepramine (Lomont®, Gamanil®), melitracen (Dixeran®, Melixeran®, Trausabun®), nitroxazepine (Sintamil®), nortriptyline (Pamelor®, Aventyl®), noxiptiline (Agedal®, Elronon®, Nogedal®), opipramol (Insidon®), pipofezine (Azafen®/Azaphen®), protriptyline (Vivactil®), trimipramine (Surmontil®), butriptyline (Evadyne®), demexiptiline (Deparon®, Tinoran®), fluacizine (Phtorazisin®), imipraminoxide (Imiprex®, Elepsin®), iprindole (Prondol®, Galatur®, Tertran®), metapramine (Timaxel®), propizepine (Depressin®, Vagran®), quinupramine (Kinupril®, Kevopril®), tiazesim (Altinil®), tofenacin (Elamol®, Tofacine®), amineptine (Survector®, Maneon®), tianeptine (Stablon®, Coaxil®); tetracyclic antidepressants such as amoxapine (Asendin®), maprotiline (Ludiomil®), mianserin (Bolvidon®, Norval®, Tolvon®), mirtazapine (Remeron®), setiptiline (Tecipul®), mianserin, mirtazapine, setiptiline; monoamine oxidase inhibitors (MAOIs) such as isocarboxazid (Marplan®), phenelzine (Nardil®), tranylcypromine (Parnate®), benmoxin (Neuralex®), iproclozide (Sursum®), iproniazid (Marsilid®), mebanazine (Actomol®), nialamide (Niamid®), octamoxin (Ximaol®), pheniprazine (Catron®), phenoxypropazine (Drazine®), pivhydrazine (Tersavid®), safrazine (Safra®), selegiline (Eldepryl®, Zelapar®, Emsam®), caroxazone (Surodil®, Timostenil®), metralindole (Inkazan®), moclobemide (Aurorix®, Manerix®), pirlindole (Pirazidol®), toloxatone (Humoryl®), eprobemide (Befol®), minaprine (Brantur®, Cantor®), bifemelane (Alnert®, Celeport®); atypical antipsychotics such as amisulpride (Solian®), lurasidone (Latuda®), quetiapine (Seroquel®); and N-methyl D-aspartate (NMDA) antagonists such ketamine (Ketalar®).

Combinations may be administered either concomitantly, e.g., as an admixture, separately but simultaneously or concurrently; or sequentially. This includes presentations in which the combined agents are administered together as a therapeutic mixture, and also procedures in which the combined agents are administered separately but simultaneously, e.g., as through separate intravenous lines into the same individual. Administration “in combination” further includes the separate administration of one of the compounds or agents administered first, followed by the second. The regimen selected can be administered concurrently since activation of the aminosterol induced response does not require the systemic absorption of the aminosterol into the bloodstream and thus eliminates concern over the likelihood systemic of drug-drug interactions between the aminosterol and the administered drug.

### VI. Definitions

The following definitions are provided to facilitate understanding of certain terms used throughout this specification.

Technical and scientific terms used herein have the meanings commonly understood by one of ordinary skill in the art, unless otherwise defined. Any suitable materials and/or methodologies known to those of ordinary skill in the art can be utilized in carrying out the methods described herein.

As used in the description of the invention and the appended claims, the singular forms “a”, “an” and “the” are used interchangeably and intended to include the plural forms as well and fall within each meaning, unless the context clearly indicates otherwise. Also, as used herein, “and/or” refers to and encompasses any and all possible combinations of one or more of the listed items, as well as the lack of combinations when interpreted in the alternative (“or”).

As used herein the term “aminosterol” refers to an amino derivative of a sterol. Non-limiting examples of suitable aminosterols for use in the composition and methods disclosed herein are Aminosterol 1436, squalamine, aminosterols isolated from Squalus acanthias, and isomers, salts, and derivatives each thereof.

The term “administering” as used herein includes prescribing for administration as well as actually administering, and includes physically administering by the subject being treated or by another.

As used herein “subject,” “patient,” or “individual” refers to any subject, patient, or individual, and the terms are used interchangeably herein. In this regard, the terms “subject,” “patient,” and “individual” includes mammals, and, in particular humans. When used in conjunction with “in need thereof,” the term “subject,” “patient,” or “individual” intends any subject, patient, or individual having or at risk for a specified symptom or disorder.

As used herein, the phrase “therapeutically effective” or “effective” in context of a “dose” or “amount” means a dose or amount that provides the specific pharmacological effect for which the compound or compounds are being administered. It is emphasized that a therapeutically effective amount will not always be effective in achieving the intended effect in a given subject, even though such dose is deemed to be a therapeutically effective amount by those of skill in the art. For convenience only, exemplary dosages are provided herein. Those skilled in the art can adjust such amounts in accordance with the methods disclosed herein to treat a specific subject suffering from a specified symptom or disorder. The therapeutically effective amount may vary based on the route of administration and dosage form.

The terms “treatment,” “treating,” or any variation thereof includes reducing, ameliorating, or eliminating (i) one or more specified symptoms and/or (ii) one or more symptoms or effects of a specified disorder. The terms “prevention,” “preventing,” or any variation thereof includes reducing, ameliorating, or eliminating the risk of developing (i) one or more specified symptoms and/or (ii) one or more symptoms or effects of a specified disorder

### EXAMPLES

### Example 1

This example describes a method of treating and/or preventing symptoms of Parkinson's disease (PD) in a clinical trial setting. The methods used in Example 1 to determine the dose of aminosterol may be used to determine the aminosterol dose in subsequent examples relating to depression or symptoms of depression.

Overview:

The subjects of the trial all had PD and experienced constipation, which is a characteristic of PD. The primary objectives of the trial involving patients with PD and constipation were to evaluate the safety and pharmacokinetics of oral squalamine (ENT-01) and to identify the dose required to improve bowel function, which was used as a clinical endpoint.

Several non-constipation PD symptoms were also assessed as endpoints, including, for example, (1) sleep problems, including daytime sleepiness; (2) non-motor symptoms, such as (i) depression (including apathy, anxious mood, as well as depression), (ii) cognitive impairment (e.g., using trail making test and the UPDRS), (iii) hallucinations (e.g., using The University of Miami Parkinson's Disease Hallucinations Questionnaire (UM-PDHQ) and the UPDRS, (iv) dopamine dysregulation syndrome (UPDRS), (v) pain and other sensations, (vi) urinary problems, (vii) light headedness on standing, and (viii) fatigue (e.g., using Parkinson's Disease Fatigue Scale 9PFS-1t and the UPDRS); (3) motor aspects of experiences of daily living, such as (i) speech, (ii) saliva and drooling, (iii) chewing and swallowing, (iv) eating tasks, (v) dressing, (vi) hygiene, (vii) handwriting; (viii) doing hobbies and other activities, (ix) turning in bed, (x) tremor, (xi) getting out of bed, a car, or a deep chair, (xii) walking and balance, (xiii) freezing; (4) motor examination, such as (i) speech, (ii) facial expression, (iii) rigidity, (ix) finger tapping, (v) hand movements, (vi) pronation-supination movements of hands, (vii) toe tapping, (viii) leg agility, arising from chair, (ix) gait, (x) freezing of gait, (xi) postural stability, (xii) posture, (xiii) global spontaneity of movement (body bradykinesia), (xiv) postural tremor of the hands, (xv) kinetic tremor of the hands, (xvi) rest tremor amplitude, (xvii) constancy of rest tremor; (5) motor complications, such as (i) time spent with dyskinesias, (ii) functional impact of dyskinesias, (iii) time spent in the off state, (iv) functional impact of fluctuations, (v) complexity of motor fluctuations, and (vi) painful off-state dystonia.

Active Agent & Dosing:

Squalamine (ENT-01; Enterin, Inc.) was formulated for oral administration in the trial. The active ion of ENT-01, squalamine, an aminosterol originally isolated from the dogfish shark, has been shown to reverse gastrointestinal dysmotility in several mouse models of PD. In addition, ENT-01 has been shown to inhibit the formation of aggregates of αS both in vitro, and in a C. elegans model of PD in vivo (Perni et al. 2017). In the C. elegans model, squalamine produced a complete reversal of muscle paralysis.

ENT-01 is the phosphate salt of squalamine. For this study it has been formulated as a small 25 mg coated tablet. Dosing ranged from 25 mg to 250 mg, with dosages greater than 25 mg requiring multiple pills (e.g., 50 mg=two 25 mg pills). Dosing instructions=take 60 mins before breakfast with 8 oz. water. The dose was taken by each patient upon awakening on an empty stomach along with 8 oz. of water simultaneously to dopamine. The subject was not allowed to ingest any food for at least 60 minutes after study medication. The compound is highly charged and will adsorb to foodstuffs, so it was administered prior to feeding.

The phosphate salt of squalamine (ENT-01) is weakly soluble in water at neutral pH but readily dissolves at pH<3.5 (the pH of gastric fluid). Squalamine, as the highly water soluble dilactate salt has been extensively studied in over three Phase 1 and eight Phase 2 human clinical trials as an intravenous agent for the treatment of cancer and diabetic retinopathy. The compound is well tolerated in single and repeat intravenous administration, alone or in combination with other agents, to doses of at least 300 mg/m²).

In the current clinical trial, squalamine (ENT-01) was administered orally to subjects with PD who have long standing constipation. Although this trial was the first in man oral dosing study of ENT-01, humans have long been exposed to low doses of squalamine (milligram to microgram) in the various commercial dogfish shark liver extracts available as nutraceuticals (e.g., Squalamax). In addition, following systemic administration squalamine is cleared by the liver and excreted as the intact molecule (in mice) into the duodenum through the biliary tract. Drug related GI toxicology has not been reported in published clinical trials involving systemic administration of squalamine.

Squalamine (ENT-01) has limited bioavailability in rats and dogs. Based on measurement of portal blood concentrations following oral dosing of radioactive ENT-01 to rat's absorption of ENT-01 from the intestine is low. As a consequence, the principal focus of safety is on local effects on the gastrointestinal tract. However, squalamine (ENT-01) appears to be well tolerated in both rats and dogs.

The starting dose in the Stage 1 segment of the trial was 25 mg (0.33 mg/kg for a 75 kg subject). The maximum single dose in Stage 1 was 200 mg (2.7 mg/kg for a 75 kg subject). The maximum dose evaluated in Stage 2 of the trial was 250 mg/day (3.3 mg/kg/day for a 75 kg subject), and the total daily dosing exposure lasted no longer than 25 days.

The daily dosing range in the clinical trial was from 25 mg (14.7 mg/m²) to 250 mg (147 mg/m²). Oral dosing of squalamine (ENT-01), because of its low oral bioavailability, is not anticipated to reach significant plasma concentrations in human subjects. In preclinical studies, squalamine (ENT-01) exhibited an oral bioavailability of about 0.1% in both rats and dogs. In Stage 1 of this phase 2 study, oral dosing up to 200 mg (114 mg/m²) yielded an approximate oral bioavailability of about 0.1%, based on a comparison of a pharmacokinetic data of the oral dosing and the pharmacokinetic data measured during prior phase 1 studies of IV administration of squalamine.

Study Protocol:

The multicenter Phase 2 trial was conducted in two Stages: a dose-escalation toxicity study in Stage 1 and a dose range-seeking and proof of efficacy study in Stage 2.

PD symptoms were assessed using a number of different tools:

(1) Numeric Rating Scales for Pain and Swelling (scale of 0-10, with 0=no pain and 10=worst pain ever experienced);

(2) Rome-IV Criteria for Constipation (7 criteria, with constipation diagnosis requiring two or more of the following: (i) straining during at least 25% of defecations, (ii) lumpy or hard stools in at least 25% of defecations, (iii) sensation of incomplete evacuation for at least 25% of defecations, (iv) sensation of anorectal obstruction/blockage for at least 25% of defecations; (v) manual maneuvers to facilitate at least 25% of defecations; (vi) fewer than 3 defecations per week; and (vii) loose stools are rarely present without the use of laxatives;

(3) Constipation—Ease of Evacuation Scale (from 1-7, with 7=incontinent, 4=normal, and 1=manual disimpaction);

(4) Bristol Stool Chart, which is a patient-friendly means of categorizing stool characteristics (assessment of stool consistency is a validated surrogate of intestinal motility) and Stool Diary;

(5) Sleep Diary (participants completed a sleep diary on a daily basis throughout the study. The diaries included time into bed and estimated time to sleep as well as wake time and duration during the night.);

(6) I-Button Temperature Assessment. The I-Button is a small, rugged self-sufficient system that measures temperature and records the results in a protected memory section. The Thermochron I-Button DS1921H (Maxim Integrated, Dallas, Tex.) was used for skin temperature measurement. I-Buttons were programmed to sample every 10 mins., and attached to a double-sided cotton sport wrist band using Velcro, with the sensor face of the I-Button placed over the inside of the wrist, on the radial artery of the dominant hand. Subjects removed and replaced the data logger when necessary (i.e., to have a bath or shower). The value of skin temperature assessment in sleep research is that the endogenous skin warming resulting from increased skin blood flow is functionally linked to sleep propensity. From the collected data, the mesor, amplitude, acrophase (time of peak temperature), Rayleight test (an index of interdaily stability), mean waveforms are calculated.);

(7) Non-motor Symptoms Questionnaire (NMSQ);

(8) Beck Depression Inventory (BDI-II);

(9) Unified Parkinson's Disease Rating Scale (UPDRS), which consists of 42 items in four subscales (Part I=Non-Motor Aspects of Experiences of Daily Living (nM-EDL) (1.1 cognitive impairment, 1.2 hallucinations and psychosis, 1.3 depressed mood, Part II=Motor Aspects of Experiences of Daily Living (M-EDL), Part III=Motor Examination, and Part IV=Motor Complications;

(10) Mini Mental State Examination (MMSE);

(11) Trail Making Test (TMT) Parts A and B;

(12) The University of Miami Parkinson's Disease Hallucinations Questionnaire (UM-PDHQ);

(13) Parkinson's Disease Fatigue Scale (PFS-16);

(14) Patient Assessment of Constipation Symptoms (PAC-SYM);

(15) Patient Assessment of Constipation Quality of Life (PAC-QOL);

(16) REM Sleep Behavior Disorder Screening Questionnaire; and

(17) Parkinson's Disease Sleep Scale.

Exploratory end-points, in addition to constipation, included for example, (i) depression assessed using the Beck Depression Inventory (BDI-II) (Steer et al. 2000) and Unified Parkinson's Disease Rating Scale (UPDRS); (ii) cognition assessed using the Mini Mental State Examination (MMSE) (Palsteia et al. 2018), Unified Parkinson's Disease Rating Scale (UPDRS), and Trail Making Test (TMT); (iii) sleep and REM-behavior disorder (RBD) using a daily sleep diary, I-Button Temperature Assessment, a REM sleep behavior disorder (RBD) questionnaire (RBDQ) (Stiasny-Kolster et al. 2007), and the UPDRS; (iv) hallucinations assessed using the PD hallucinations questionnaire (PDHQ) (Papapetropoulos et al. 2008), the UPDRS, and direct questioning; (v) fatigue using the Parkinson's Disease Fatigue Scale (PFS-16) and the UPDRS; (vi) motor functions using the UPDRS; and (vii) non-motor functions using the UPDRS.

Assessments were made at baseline and at the end of the fixed dose and washout periods. Circadian system status was evaluated by continuously monitoring wrist skin temperature (Thermochron iButton DS1921H; Maxim, Dallas) following published procedures (Sarabia et al. 2008).

Based on these data, it is believed that administration of squalamine (ENT-01), a compound that can displace αS from membranes in vitro, reduces the formation of neurotoxic αS aggregates in vivo, and stimulates gastrointestinal motility in patients with PD and constipation. The observation that the dose required to achieve a prokinetic response increases with constipation severity supports the hypothesis that the greater the burden of αS impeding neuronal function, the higher the dose of squalamine (ENT-01) required to restore normal bowel function.

Study Design:

A multicenter Phase 2 trial was conducted in two Stages: a dose-escalation toxicity study in Stage 1 and a dose range-seeking and proof of efficacy study in Stage 2. The protocol was reviewed and approved by the institutional review board for each participating center and patients provided written informed consent.

Following successful screening, all subjects underwent a 14-day run-in period where the degree of constipation was assessed through a validated daily log (Zinsmeister et al. 2013) establishing baseline CSBMs/week. Subjects with an average of <3 CSBMs/week proceeded to dosing.

In Stage 1, ten (10) PD patients received a single escalating dose of squalamine (ENT-01) every 3-7 days beginning at 25 mg and continuing up to 200 mg or the limit of tolerability, followed by 2-weeks of wash-out. Duration of this part of the trial was 22-57 days. The 10 subjects in the sentinel group were assigned to Cohort 1 and participated in 8 single dosing periods. Tolerability limits included diarrhea or vomiting. A given dose was considered efficacious in stimulating bowel function (prokinetic) if the patient had a complete spontaneous bowel movement (CSBM) within 24 hours of dosing.

Each dose period was staggered, so that subjects 1-2 were administered a single dose of the drug at the lowest dose of 25 mg. Once 24 hours have elapsed, and provided there are no safety concerns, the patient was sent home and brought back on day 4-8 for the next dose. During the days the subjects are home, they completed the daily diaries and e-mailed them to the study coordinators. Subjects 3-10 were dosed after the first 2 subjects have been observed for 72 hours, i.e. on Day 4. Subjects 1-2 were also brought back on Day 4-8 and given a single dose of 50 mg. Once another 24 hours have elapsed and provided there are no safety concerns, the patients were all sent home and instructed to return on Day 7 for the next dosing level. This single dosing regimen was continued until each subject was administered a single dose of 200 mg or has reached a dose limiting toxicity (DLT). DLT was the dose which induces repeated vomiting, diarrhea, abdominal pain or symptomatic postural hypotension within 24 hours of dosing.

In Stage 2, 34 patients were evaluated. First, 15 new PD patients were administered squalamine (ENT-01) daily, beginning at 75 mg, escalating every 3 days by 25 mg to a dose that had a clear prokinetic effect (CSBM within 24 hours of dosing on at least 2 of 3 days at a given dose), or the maximum dose of 175 mg or the tolerability limit. This dose was then maintained (“fixed dose”) for an additional 3-5 days. After the “fixed dose”, these patients were randomly assigned to either continued treatment at that dose or to a matching placebo, for an additional 4-6 days prior to a 2-week wash-out.

A second cohort of 19 patients received squalamine (ENT-01) escalating from 100 mg/day to a maximum of 250 mg/day without subsequent randomization to squalamine (ENT-01) or placebo. Criteria for dose selection and efficacy were identical to those used in the previous cohort.

Patient Population:

Patients were between 18 and 86 years of age and diagnosed with PD by a clinician trained in movement disorders following the UK Parkinson's Disease Society Brain Bank criteria (Fahn et al. 1987). Patients were required to have a history of constipation as defined by <3 CSBMs/week and satisfy the Rome IV criteria for functional constipation (Mearin et al. 2016) at screening, which requires 2 or more of the following: Straining during at least 25% of defecations; lumpy or hard stools in at least 25% of defecations; sensation of incomplete evacuation in at least 25% of defecations; sensation of anorectal obstruction/blockage in at least 25% of defecations; and/or manual maneuvers to facilitate at least 25% of defecations.

Baseline characteristics of patients are shown in Table 2. Patients in Stage 2 had somewhat longer duration of Parkinson's disease and higher UPDRS scores than participants in Stage 1.

TABLE 2
Baseline Characteristics of Dosed Patients

| | | | |
|---|---|---|---|
| | Stage 1** | Stage 2*** | Total |
| Characteristic | (n = 10) | (n = 34) | (n = 44) |

Sex- no. (%)

| | | | |
|---|---|---|---|
| Male | 5 (50) | 25 (73.5) | 30 (68.1) |
| Female | 5 (50) | 9 (26.5) | 14 (31.8) |
| White race-no. (%) | 8 (80) | 34 (100) | 42 (95.54) |

Age-yr

| | | | |
|---|---|---|---|
| Mean | 65.0 | 74.5 | 72.5 |
| Range | 58-70.5 | 60.6-84.2 | 58-84.2 |

Age at PD diagnosis-yr

| | | | |
|---|---|---|---|
| Mean | 61.1 | 67.7 | 66.2 |
| Range | 54.2-69 | 50.6-82.5 | 50.6-82.5 |

Duration of PD-yr

| | | | |
|---|---|---|---|
| Mean | 4.2 | 6.8 | 6.2 |
| Range | 1-11 | 0.3-17.3 | 0.3-17.3 |

Duration of constipation-yr

| | | | |
|---|---|---|---|
| Mean | 25.8 | 16.8 | 18.9 |
| Range | 1-65 | 0.5-66.0 | 0.5-66.0 |

UPDRS score

| | | | |
|---|---|---|---|
| Mean | 53.4 | 63.2 | 61.3 |
| Range | 33-88 | 24-122 | 24.0-122.0 |

Hoehn and Yahr-Stage

| | | | |
|---|---|---|---|
| Mean | 2.0 | 2.4 | 2.3 |
| Range | 2.0 | 1.0-5.0 | 1.0-5.0 |

Constipation severity*-
CSBM/wk- no. (%)

| | | | |
|---|---|---|---|
| 0-1 | 8(80) | 14(41.2) | 22 (50) |
| 1.1-2 | 2 (20) | 17(50) | 19 (43.2) |
| 2.1-3 | 0 | 3 (8.8) | 3 (6.8) |

*At baseline. Baseline value is the average number of CSBMs per week calculated at the end of the 2-week run-in period.
**In Stage 1, 10 patients received single escalating doses every 3-7 days starting at 25 mg and escalating up to dose limiting toxicity (DLT) or 200 mg, whichever came first, followed by a 2-week wash-out period.
***In Stage 2, 15 patients received daily doses starting at 75 mg and escalating every 3 days up to prokinetic dose (dose producing CSBMs on at least 2 of 3 days) or 175 mg, whichever came first, followed by an additional 2-4 days at that dose (“fixed dose” period) and were then randomized to treatment at the “fixed-dose” or placebo for 4-6 days. Wash-out lasted 2 weeks. The remaining 19 patients were escalated from 100 mg to prokinetic dose or 250 mg, whichever came first, followed by an additional 2-4 days at that dose and then a 2-week wash-out period.

Safety and Adverse Event (AE) Profile:

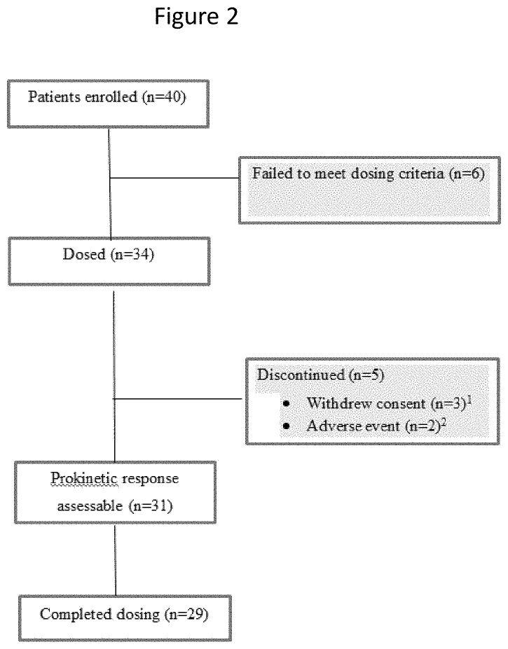

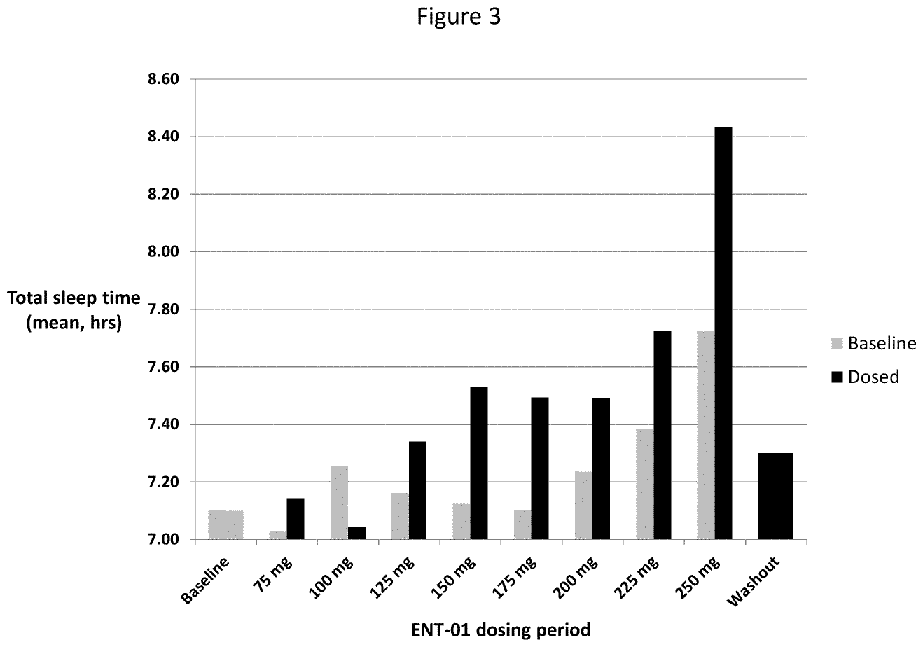

Fifty patients were enrolled and 44 were dosed. In Stage 1, 10 patients were dosed, 1 (10%) withdrew prior to completion and 9 (90%) completed dosing. In stage 2, 6 (15%) patients had ≥3 CSBM/week at the end of the run-in period and were excluded, 34 patients were dosed and bowel response was assessable in 31 (91%). Two patients (5.8%) were terminated prior to completion because of recurrent dizziness, and 3 others withdrew during dosing (8.8%): 2 because of diarrhea and 1 because of holiday. Fifteen patients were randomized. Study-drug assignments and patient disposition are shown in Table 3 and FIG. 2.

TABLE 3
Study drug assignments and adherence to treatment

| | Stage 1 | Stage 2 | |
|---|---|---|---|
| Enrolled | 10 | 40 | |
| Failed prior to dosing | 0 | 6 | |
| Dosed | 10 | 34 | |
| 25-200 mg | 10 | | |
| 75-175 mg | | 19 | |
| 100-250 mg | | 15 | |
| Terminated (%) | 0 (0) | 2* | (5.8) |
| Withdrew (%) | 1 (10) | 3 | (8.8) |
| Completed dosing (%) | 9 (90) | 31** | (91) |

| | |
|---|---|
| Randomized | 15 |
| Treatment | 6 |
| Placebo | 9 |

The 2 patients who were terminated **29 patients completed dosing but an additional 2 who withdrew had an assessable prokinetic end-point.

Most AEs were confined to the GI tract (88% in Stage 1 and 63% in Stage 2). The most common AE was nausea which occurred in 4/10 (40%) patients in Stage 1 and in 18/34 (52.9%) in Stage 2 (Table 2). Diarrhea occurred in 4/10 (40%) patients in Stage 1 and 15/34 (44%) in Stage 2. One patient withdrew because of recurrent diarrhea. Other GI related AEs included abdominal pain 11/44 (32%), flatulence 3/44 (6.8%), vomiting 3/44 (6.8%), worsening of acid reflux 2/44 (4.5%), and worsening of hemorrhoids 1/44 (2.2%). One patient had a lower GI bleed (Serious adverse event, SAE) during the withdrawal period. This patient was receiving aspirin, naproxen and clopidogrel at the time of the bleed, and colonoscopy revealed large areas of diverticulosis and polyps. This SAE was considered unrelated to study medication. The only other noteworthy AE was dizziness 8/44 (18%). Dizziness was graded as moderate in one patient who was receiving an alpha-adrenergic blocking agent (Terazosin). This patient was withdrawn from the study and recovered spontaneously. All other AEs resolved spontaneously without discontinuation of squalamine (ENT-01). The relationship between dose and AEs is shown in Table 4.

TABLE 4
All adverse events (n, %)

| | Stage 1 | Stage 2 |
|---|---|---|
| Enrolled | (n = 10) | (n = 40) |
| Dosed | 10 | 34 |

GI:
Nausea

| | | |
|---|---|---|
| Mild | 4(40) | 18(52) |
| Moderate | 0 | 1(2.9) |

Diarrhea

| | | |
|---|---|---|
| Mild | 1(10) | 12(35) |
| Moderate | 3(30) | 2(5.8) |
| Severe | 0 | 1(2.9) |

Vomiting

| | | |
|---|---|---|
| Mild | 1(10) | 2(5.8) |
| Moderate | 0 | 0 |

Abdominal pain

| | | |
|---|---|---|
| Mild | 2(20) | 4(11.7) |
| Moderate | 3(30) | 2(5.8) |

Flatulence

| | | |
|---|---|---|
| Mild | 2(20) | 1(3) |
| Moderate | 0 | 0 |

Loss of appetite*

| | | |
|---|---|---|
| Mild | 1(10) | 0 |
| Moderate | 0 | 0 |

Worsening acid reflux

| | | |
|---|---|---|
| Mild | 0 | 4(11.7) |
| Moderate | 0 | 0 |

Worsening hemorrhoid

| | | |
|---|---|---|
| Mild | 0 | 1(3) |
| Moderate | 0 | 0 |

Lower GI bleed**

| | | |
|---|---|---|
| Severe | 0 | 1(2.5) |

Non-GI:
Dizziness

| | | |
|---|---|---|
| Mild | 0 | 7(20.5) |
| Moderate | 0 | 1(2.9) |

Blood in urine*

| | | |
|---|---|---|
| Mild | 1(10) | 0 |
| Moderate | 0 | 0 |

Headache

| | | |
|---|---|---|
| Mild | 1(10) | 3(8.8) |
| Moderate | 0 | 0 |

Urinary retention

| | | |
|---|---|---|
| Mild | 0 | 1(3) |
| Moderate | 0 | 0 |

Urinary tract infection

| | | |
|---|---|---|
| Mild | 0 | 1(3) |
| Moderate | 0 | 2(5.8) |

Increased urinary frequency

| | | |
|---|---|---|
| Mild | 0 | 2(5.8) |
| Moderate | 0 | 0 |

Skin lesions-rash

| | | |
|---|---|---|
| Mild | 0 | 3(8.8) |
| Moderate | 0 | 0 |

Eye infection

| | | |
|---|---|---|
| Mild | 0 | 1(3) |
| Moderate | 0 | 0 |

Difficulty falling asleep

| | | |
|---|---|---|
| Mild | 0 | 1(3) |
| Moderate | 0 | 0 |

*Unrelated to ENT-01
**colonic diverticulosis, polyp, patient on aspirin, Plavix and naproxen. Unrelated to ENT-01

TABLE 5
Common adverse events by dose

| Dose | Stage 1 | | | Stage 2 | | |
|---|---|---|---|---|---|---|
| (mg) | Diarrhea | Nausea | Vomiting | Diarrhea | Nausea | Dizziness* |
| 0 | 0 | 0 | 0 | 1 | 0 | 2 |
| 25 | 1 | 0 | 0 | — | — | — |
| 50 | 1 | 0 | 0 | — | — | — |
| 75 | 1 | 0 | 0 | 7 | 3 | 8 |
| 100 | 0 | 1 | 1 | 10 | 12 | 7 |
| 125 | 1 | 2 | 1 | 3 | 4 | 8 |
| 150 | 1 | 0 | 0 | 2 | 11 | 2 |
| 175 | 1 | 1 | 0 | 1 | 12 | 0 |
| 200 | 0 | 2 | 0 | 3 | 6 | — |
| 225 | — | — | — | 3 | 1 | |
| 250 | — | — | — | 2 | — | |

*lightheadedness included

TABLE 6
Dose limiting toxicity criteria

| | |
|---|---|
| Diarrhea | Increase 4-6 stools/day over baseline |
| Vomiting | 3-5 episodes in 24 hours |
| Abdominal pain | Moderate pain limiting daily activities |
| Postural hypotension | Moderately symptomatic and limiting daily |
| | activities or BP < 80/40 |

No formal sample size calculation was performed for Stage 1. The number of subjects (n=10) was based on feasibility and was considered sufficient to meet the objectives of the study; which was to determine the tolerability of the treatment across the range of tested doses. For Stage 2, assuming the highest proportion of spontaneous resolution of constipation with no treatment to be 0.10, 34 evaluable subjects who have measurements at both baseline and at the end of the fixed dose period provided 80% power to detect the difference between 0.10 (proportion expected if patients are not treated) and a squalamine (ENT-01) treated proportion of 0.29.

No randomization was performed for Stage 1. During the randomization period of Stage 2, subjects were randomly allocated in equal proportion (1:1) to 1 of 2 double-blind treatment groups in a block size of 4: (1) squalamine (ENT-01) at the identified fixed dose level, or (2) placebo at the identified fixed dose level.

Adverse events were coded using the current version of MedDRA. Severity of AEs were assessed by investigators according to CTCAE (v4.03): Grade 1 is labeled as Mild, Grade 2 as Moderate, and Grade 3 and above as Severe. AEs that have a possible, probable or definite relationship to study drug were defined to be related to the study drug while others were defined as “not related”. The number (percentage) of subjects who experienced an AE during escalation and fixed dosing periods were summarized by dose level and overall for each stage. The denominator for calculating the percentages were based on the number of subjects ever exposed to each dose and overall.

Effect on Bowel Function:

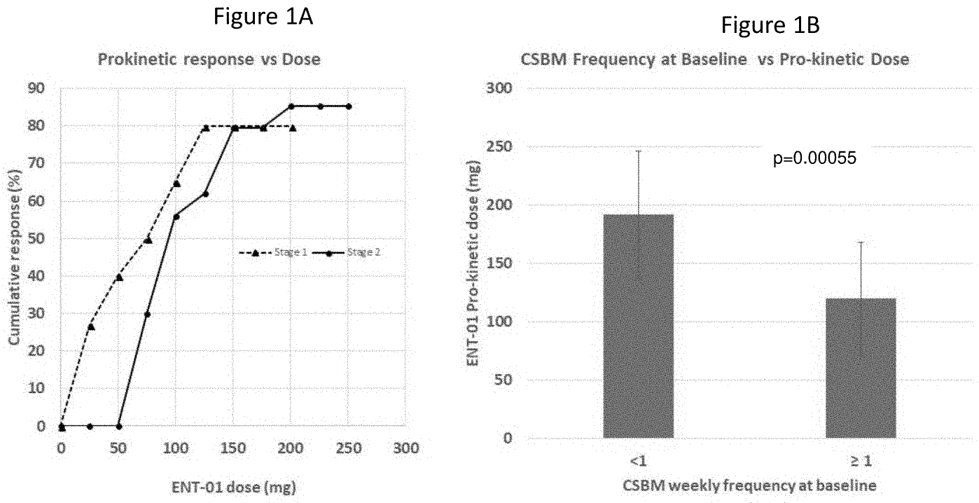

Cumulative responder rates of bowel function are shown in FIG. 1A. In Stage 1 (single dose), cumulative response rate increased in a dose-dependent fashion from 25% at 25 mg to a maximum of 80% at 200 mg.

In Stage 2 (daily dosing), the response rate increased in a dose-dependent fashion from 26% at 75 mg to 85.3% at 250 mg. The dose required for a bowel response was patient-specific and varied from 75 mg to 250 mg. Median efficacious dose was 100 mg. Average CSBM/week increased from 1.2 at baseline to 3.8 at fixed dose (p=2.3×10⁻⁸) and SBM increased from 2.6 at baseline to 4.5 at fixed dose (p=6.4×10⁻⁶) (Table 7). Use of rescue medication decreased from 1.8/week at baseline to 0.3 at fixed dose (p=1.33×10⁻⁵). Consistency based on the Bristol stool scale also improved, increasing from mean 2.7 to 4.1 (p=0.0001) and ease of passage increased from 3.2 to 3.7 (p=0.03). Subjective indices of wellbeing (PAC-QOL) and constipation symptoms (PAC-SYM) also improved during treatment (p=0.009 and p=0.03 respectively).

TABLE 7
Stool related indices Stage 2 (Dosed patients, n = 34)

| | Baseline | Fixed dose | |
|---|---|---|---|
| | (mean, SD) | (mean, SD) | P-value |
| CSBM* | 1.2 (0.90) | 3.8 (2.40) | 2.3 × 10⁻⁸ |
| SBM* | 2.6 (1.45) | 4.5 (2.21) | 6.4 × 10⁻⁶ |
| Suppository use* | 1.8 (1.92) | 0.3 (0.67) | 1.33 × 10⁻⁵ |
| Consistency*** | 2.7 (1.20) | 4.1 (2.13) | 0.0001 |
| Ease of passage** | 3.2 (0.73) | 3.7 (1.19) | 0.03 |
| PAC-QOL total | 1.4 (0.49) | 1.2 (0.59) | 0.009 |
| PAC-SYM | 1.3 (0.45) | 1.1 (0.49) | 0.03 |

*weekly average;
**Ease of evacuation scale, where 1-manual disimpaction and 7 = incontinent;
***Bristol stool scale 1-7, where 1 = separate hard lumps and 7 = liquid consistency

The dose that proved efficacious in inducing a bowel response was strongly related to constipation severity at baseline (p=0.00055) (FIG. 1B); patients with baseline constipation of <1 CSBM/week required higher doses for a response (mean 192 mg) than patients with ≥1 CSBM/week (mean 120 mg).

While the improvement in most stool-related indices did not persist beyond the treatment period, CSBM frequency remained significantly above baseline value (Table 8).

TABLE 8
Reversal of stool indices to baseline
during the wash-out period (Stage 2)

| | | | | P-value |
|---|---|---|---|---|
| | Baseline | Fixed dose | Wash-out | (wash-out vs. |
| | (Mean, SD) | (Mean, SD) | (Mean, SD) | baseline) |
| CSBM | 1.2 (0.90) | 3.8 (2.4) | 1.8 (1.19) | 0.01 |
| SBM | 2.6 (1.45) | 4.5 (2.21) | 3.2 (1.80) | 0.16 |
| Ease | 3.2 (0.73) | 3.7 (1.19) | 3.3 (0.81) | 0.78 |
| Consistency | 2.7 (1.20) | 4.1 (2.13) | 2.8 (1.39) | 0.85 |
| Rescue meds | 1.8 (1.92) | 0.3 (0.67) | 1.0 (1.40) | 0.13 |
| PAQ-QOL | 1.4 (0.49) | 1.2 (0.59 | 1.2 (0.63) | 0.04 |
| PAQ-SYM | 1.3 (0.45) | 1.1 (0.49) | 1.1 (0.60) | 0.11 |

The primary efficacy outcome variable was whether or not a subject was a “success” or “failure”. This is an endpoint based on subject diary entries for the “fixed dose” period prior to the endpoint assessment defined as average complete stool frequency increase by 1 or more over baseline, or 3 or more complete spontaneous stools/week. The subject was deemed a “success” if s/he met one or more of the criteria listed above, otherwise the subject was deemed a “failure”. The primary analysis was based on all subjects with a baseline assessment and an assessment at the end of the “fixed-dose” period and was a comparison of the proportion of successes with 0.10 (the null hypothesis corresponding to no treatment effect).

The proportion of subjects for whom the drug was a success was estimated with a binomial point estimate and corresponding 95% confidence interval. A secondary analysis compared the proportions of subjects who are deemed a success at the end of the randomized fixed-dose period between those randomized to the squalamine (ENT-01) arm and those randomized to the placebo arm. A Fisher's exact test was used to compare the proportions of subjects who were deemed a success at the end of randomization period between the two randomized arms

Subgroup Analysis:

Fifteen patients were randomized to treatment (n=6) or placebo (n=9) after the fixed dose period. During the 4-6 days of randomized treatment, the mean CSBM frequency in the treatment group remained higher than baseline as compared to those receiving placebo who returned to their baseline values (Table 9).

TABLE 9
CSBM frequency in the randomized cohort

| CSBM/week | Baseline | Fixed dose | Randomized | Washout |
|---|---|---|---|---|
| Treatment (n = 6) | 0.8 | 3.2 | 2.4 | 0.9 |
| Placebo (n = 9) | 1.6 | 3.3 | 1.4 | 1.6 |

CSBM increased in both groups during the treatment period and remained high in the treatment group during the randomized period but fell to baseline values in the placebo group.

Pharmacokinetics:

PK data were collected on the 10 patients enrolled in Stage 1 and 10 patients enrolled in Stage 2 to determine the extent of systemic absorption. In Stage 1, PK data were obtained at each visit, pre-medication, at 1, 2, 4, 8 and 24 hours (Table 10). In Stage 2, PK was measured on days 1 and 6 of the randomization period pre-medication, at 1, 2, 4 and 8 hours (Table 11). Based on the pharmacokinetic behavior of intravenously administered squalamine determined in prior clinical studies it is estimated that squalamine (ENT-01) exhibited oral bioavailability of less than 0.3% (Bhargava et al. 2001; Hao et al. 2003).

TABLE 10
Pharmacokinetics of orally administered squalamine (ENT-01) in Stage 1.
Stage 1

| | | | Tₘₐₓ | | | |
|---|---|---|---|---|---|---|
| | | Cₘₐₓ | (hour) | T_{1/2} | | AUC_{0-16 hr} |
| Dose | # of | (ng/ | (Median | (hours) | AUC_{0-8 hr} | (ng * hour/ |
| (mg) | patients | ml) | Value) | (n) | (ng * hour/ml | ml |
| 25 | 9 | 2.84 | 1.0 | 2.6 (3) | 10.8 | 19.6 |
| 50 | 10 | 3.73 | 2.0 | 3.4 (3) | 18.5 | 33.1 |
| 75 | 9 | 4.33 | 2.0 | 2.8 (2) | 18.4 | 29.8 |
| 100 | 9 | 6.18 | 2.0 | 3.9 (5) | 29.6 | 51.5 |
| 125 | 9 | 9.63 | 2.0 | 3.9 (4) | 43.1 | 77.7 |
| 150 | 7 | 6.27 | 2.0 | 5.6 (4) | 31.5 | 64.0 |
| 175 | 7 | 10.3 | 2.0 | 9.1 (6) | 49.7 | 91.2 |
| 200 | 6 | 15.1 | 2.0 | 9.0 (5) | 78.3 | 157 |

TABLE 11
Pharmacokinetics of orally administered squalamine (ENT-01) in Stage 2.
Stage 2

| | # of | | | | |
|---|---|---|---|---|---|
| | patients | | Tₘₐₓ (hour) | T_{1/2} | |
| Dose | (2 visits | Cₘₐₓ | (Median | (hours) | AUC_{0-8 hr} |
| (mg) | each) | (ng/ml) | Value) | (n) | (ng*hour/ml |
| 75 | 1 | 10.0 | 3.0 | 5.5 (1) | 59.0 |
| 100 | 4 | 17.7 | 1.0 | 4.8 (5) | 70.3 |
| 125 | | | | | |
| 150 | | | | | |
| 175 | 5 | 11.8 | 2.0 | 10 (6) | 66.8 |

The mean Cₘₐₓ, Tₘₐₓ and T_{1/2} and AUC of the squalamine ion following squalamine (ENT-01) oral dosing for Stage 1 patients. The PK analyses are only approximate, as the lower limit of the validated concentration range was 10 ng/ml; most of the measured concentrations fell below that value. The mean Cₘₐₓ, Tₘₐₓ and T_{1/2} and AUC of the squalamine ion following squalamine (ENT-01) oral dosing for Stage 2 patients. The PK analyses are only approximate, as the lower limit of the validated concentration range was 0.5 ng/ml.

CNS Symptoms in Stage 2:

An exploratory analysis was done with respect to the sleep data, the body temperature data, mood, fatigue, hallucinations, cognition and other motor and non-motor symptoms of PD. Continuous measurements within a subject were compared with a paired t-test and continuous measurements between subject groups were compared with a two-group t-test. Categorical data were compared with a chi-squared test or a Fisher's exact test if the expected cell counts are too small for a chi-squared test.

CNS Symptoms:

CNS symptoms were evaluated at baseline and at the end of the fixed dose period and the wash-out period (Table 12). Total UPDRS score was 64.4 at baseline, 60.6 at the end of the fixed dose period and 55.7 at the end of the wash-out period (p=0.002); similarly, the motor component of the UPDRS improved from 35.3 at baseline to 33.3 at the end of fixed dose to 30.2 at the end of wash-out (p=0.006). MMSE improved from 28.4 at baseline to 28.7 during treatment and to 29.3 during wash-out (p=0.0006). BDI-II decreased from 10.9 at baseline to 9.9 during treatment and 8.7 at wash-out (p=0.10). PDHQ improved from 1.3 at baseline to 1.8 during treatment and 0.9 during wash-out (p=0.03). Hallucinations were reported by 5 patients at baseline and delusions in 1 patient. Both hallucinations and delusions improved or disappeared in 5 of 6 patients during treatment and did not return for 4 weeks following discontinuation of squalamine (ENT-01) in 1 patient and 2 weeks in another. The frequency of arm or leg thrashing reported in the sleep diary diminished progressively from 2.2 episodes/week at baseline to 0 at maximal dose. Total sleep time increased progressively from 7.1 hours at baseline to 8.4 hours at 250 mg and was consistently higher than baseline beyond 125 mg (FIG. 4). Unlike stool-related indices, the improvement in many CNS symptoms persisted during wash-out.

TABLE 12
Effect of Squalamine (ENT-01) on neurological symptoms (n = 34)

| | Baseline | | Fixed dose | | | Wash-out | | |
|---|---|---|---|---|---|---|---|---|
| UPDRS | (Mean, SD) | | (Mean, SD) | | P-value | (Mean, SD) | | P-value |
| Part 1 (NMS) | 11.6 | (6.51) | 10.6 | (6.18)) | 0.28 | 9.5 | (5.27) | 0.06 |
| Part 2 (Daily | 14.9 | (8.11) | 14.7 | (9.02) | 0.77 | 14.1 | (8.21) | 0.40 |
| living) | | | | | | | | |
| Part 3 (Motor) | 35.3 | (14.35) | 33.3 | (15.20) | 0.13 | 30.2 | (13.23) | 0.005 |
| Total | 64.4 | (23.72) | 60.6 | (25.60) | 0.09 | 55.7 | (23.69) | 0.002 |
| MMSE | 28.4 | (1.75) | 28.7 | (1.9) | 0.21 | 29.3 | (1.06) | 0.0006 |
| PDHQ | 1.3 | (2.99) | 1.8 | (3.34) | 0.45 | 0.9 | (2.33) | 0.03 |
| BDI-II | 10.9 | (7.12) | 9.9 | (6.45) | 0.14 | 8.7 | (5.19) | 0.10 |

UPDRS: Unified Parkinson's Disease Severity Score;
NMS: Non-motor symptoms;
BDI: Beck Depression Index-II;
MMSE: Mini-mental State exam.
PDHQ: Parkinson's Disease Hallucination Questionnaire

Circadian rhythm of skin temperature was evaluable in 12 patients (i.e., those who had recordings that extended from baseline through washout). Circadian system functionality was evaluated by continuously monitoring wrist skin temperature using a temperature sensor (Thermochron iButton DS1921H; Maxim, Dallas, Tex.) (Sarabia et al. 2008). A nonparametric analysis was performed for each participant to characterize DST as previously described (Sarabia et al. 2008; Ortiz-Tudela et al. 2010).

Briefly, this analysis includes the following parameters: (i) the inter-daily stability (the constancy of 24-hour rhythmic pattern over days, IS); (ii) intra-daily variability (rhythm fragmentation, IV); (iii) average of 10-minute intervals for the 10 hours with the minimum temperature (L10); (iv) average of 10-minute intervals for the 5 hours with the maximum temperature (M5) and the relative amplitude (RA), which was determined by the difference between M5 and L10, divided by the sum of both. Finally, the Circadian Function Index (CFI) was calculated by integrating IS, IV, and RA. Consequently, CFI is a global measure that oscillates between 0 for the absence of circadian rhythmicity and 1 for a robust circadian rhythm (Ortiz-Tudela et al. 2010).

A comparison was performed of circadian rhythm parameters during the baseline, fixed dose and washout periods. ENT-01 administration improved all markers of healthy circadian function, increasing rhythm stability (IS, p=0.026), relative amplitude (RA, p=0.001) and circadian function index (CFI, p=0.016), while reducing rhythm fragmentation (IV, p=0.031). The improvement persisted for several of these circadian parameters during wash-out period (IS, p=0.008 and CFI, p=0.004). (FIG. 5).

### CONCLUSIONS

This Phase 2 trial involving 50 patients with PD assessed the safety of orally administered ENT-01, and the effect on bowel function and neurologic symptoms of PD. In addition, the study aimed to identify a dose of ENT-01 that normalizes bowel function in each patient. The study achieved the objectives of identifying safety and pharmacodynamics responses of ENT-01 in PD. In addition, the study is the first proof of concept demonstration that directly targeting αS pharmacologically can achieve beneficial GI, autonomic and CNS responses.

The effective dose ranged between 75 mg and 250 mg, with 85% of patients responding within this range. This dose correlated positively with constipation severity at baseline consistent with the hypothesis that gastrointestinal dysmotility in PD results from the progressive accumulation of αS in the ENS, and that squalamine (ENT-01) can restore neuronal function by displacing αS and stimulating enteric neurons. These results demonstrate that the ENS in PD is not irreversibly damaged and can be restored to normal function.

Several exploratory endpoints were incorporated into the trial to evaluate the impact of ENT-01 on neurologic symptoms associated with PD. The UPDRS score, a global assessment of motor and non-motor symptoms, showed significant improvement. Improvement was also seen in the motor component. The improvement in the motor component is unlikely to be due to improved gastric motility and increased absorption of dopaminergic medications, since improvement persisted during the 2-week wash-out period, i.e., in the absence of study drug (Table 12).

Improvements were also seen in cognitive function (MMSE scores), hallucinations, REM-behavior disorder (RBD) and sleep. Six of the patients enrolled had daily hallucinations or delusions and these improved or disappeared during treatment in five. In one patient the hallucinations disappeared at 100 mg, despite not having reached the colonic prokinetic dose at 175 mg. The patient remained free of hallucinations for 1 month following cessation of dosing. RBD and total sleep time also improved progressively in a dose-dependent manner.

The prokinetic effect of the aminosterol squalamine appears to occur through local action of the compound on the ENS, since squalamine, the active zwitterion, is not significantly absorbed into the systemic circulation.

### Example 2—Constipation

This prophetic example describes an exemplary method of (i) treating constipation associated with depression and/or (ii) treating and/or preventing depression in which constipation is a known symptom in a subject.

Depression patients are selected based on the constipation criteria described in Example 1. The patients are then subdivided into a control subgroup and a treatment subgroup. A “fixed dose” of an aminosterol or a salt or derivative thereof for each of the patients in the treatment subgroup is determined using the method described in Example 1 and in the application supra. Treatment and wash-out periods mirror Example 1. Depression patients are monitored for changes in the severity or occurrence of the symptoms. Depression patients are also monitored for changes in other symptoms associated with depression.

Depression patients having more severe constipation, e.g., less than 1 spontaneous bowel movement per week, are started at a dose of 75 mg or more. Depression patients having less severe constipation, e.g., 1 or more SBM/week, are started at a lower dose of aminosterol, e.g., a starting dose of less than 75 mg, for example a dose of 25 mg/day. Thus, the starting aminosterol dose is dependent upon constipation severity. The full aminosterol dosing range is from about 1 to about 500 mg. Once a fixed aminosterol dose has been identified for a depression patient, the depression subject is started at that same dose following drug cessation and reintroduction of drug dosing; e.g., there is no need to ramp up dosing once a fixed aminosterol dose for a patient has been identified.

### Example 3—Suicidal Thoughts

This prophetic example describes an exemplary method of (i) treating suicidal thoughts associated with depression and/or (ii) treating and/or preventing depression in which suicidal thoughts are a known symptom in a subject.

Depression patients are selected based on having suicidal thoughts. The patients are then subdivided into a control subgroup and a treatment subgroup. A “fixed dose” of an aminosterol or a salt or derivative thereof for each of the patients in the treatment subgroup is determined using the method described in Example 1, using the improvement of suicidal thoughts as an endpoint. Treatment and wash-out periods mirror Example 1. Depression patients are monitored for changes in the severity or occurrence of the symptoms.

### Example 4—Sleep Problem, Sleep Disorder, or Sleep Disturbance

This prophetic example describes an exemplary method of (i) treating sleep problem, sleep disorder, or sleep disturbance in depression patients and/or (ii) treating and/or preventing depression in which REM disturbed sleep is a known symptom in a depression subject having REM disturbed sleep.

Depression patients are selected based on having a sleep problem, sleep disorder, or sleep disturbance. The patients are then subdivided into a control subgroup and a treatment subgroup. A “fixed dose” of an aminosterol or a salt or derivative thereof for each of the depression patients in the treatment subgroup is determined using the method described in Example 1, using the improvement of sleep problem, sleep disorder, or sleep disturbance symptoms as an endpoint. Treatment and wash-out periods mirror Example 1. Depression patients are monitored for changes in the severity or occurrence of the symptoms.

### Example 5—Weight Loss

This prophetic example describes an exemplary method of (i) treating weight loss in a depression subject and/or (ii) treating and/or preventing depression in which weight loss is a known symptom (a weight loss associated disorder, for example depression) in a depression subject having displaying weight loss.

Depression patients are selected based on having circadian rhythm dysfunction. The patients are then subdivided into a control subgroup and a treatment subgroup. A “fixed dose” of an aminosterol or a salt or derivative thereof for each of the depression patients in the treatment subgroup is determined using the method described in Example 1, using either the improvement of weight loss symptoms as an endpoint. Treatment and wash-out periods mirror Example 1. Patients are monitored for changes in the severity or occurrence of the symptoms.

### Example 6—Depression

This prophetic example describes an exemplary method of treating and/or preventing depression in a subject in need thereof.

Depression patients are selected based on being diagnosed with depression, i.e., having depression, or exhibiting known risk factors of depression, i.e., at risk for developing depression. Patients are grouped based on having depression or at risk for developing depression. The groups are then subdivided into a control subgroup and a treatment subgroup. A “fixed dose” of an aminosterol or a salt or derivative thereof for each of the patients in the treatment subgroup is determined using the method described in Example 1, using either the improvement of constipation or another symptom of depression as an endpoint. Treatment and wash-out periods mirror Example 1. Patients are monitored for changes in the severity or occurrence of the symptoms. Patients having depression are monitored for changes in other symptoms associated with the disorder. Patients at risk for developing depression are monitored for the development of depression.

### Example 7—Cognitive Impairment

This prophetic example describes an exemplary method of (i) treating cognitive impairment in depression subjects/or (ii) treating and/or preventing depression in which cognitive impairment is a known symptom.

Depression patients are selected based on having cognitive impairment. The patients are then subdivided into a control subgroup and a treatment subgroup. A “fixed dose” of an aminosterol or a salt or derivative thereof for each of the patients in the depression treatment subgroup is determined using the method described in Example 1, using the improvement of cognitive impairment symptoms as an endpoint. Treatment and wash-out periods mirror Example 1. Patients are monitored for changes in the severity or occurrence of the symptoms. Depression patients are also monitored for changes in other symptoms associated with depression.

While certain embodiments have been illustrated and described, it should be understood that changes and modifications can be made therein in accordance with ordinary skill in the art without departing from the technology in its broader aspects as defined in the following claims.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, or compositions, which can of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof, inclusive of the endpoints. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims

### REFERENCES

Aarsland et al., “Neuropsychiatric symptoms in patients with Parkinson's disease and dementia: frequency, profile and associated care giver stress,” J. Neurol. Neurosurg. Psychiatry, 78:36-42 (2007).
Acsadi et al., “Alpha-synuclein loss in spinal muscular atrophy,” J. Mol. Neurosci., 43(3):275-83 (2011).
Albert et al., “The Diagnosis of Mild Cognitive Impairment Due to Alzheimer's Disease: Recommendations from the National Institute on Aging-Alzheimer's Association Workgroups on Diagnostic Guidelines for Alzheimer's Disease,” Alzheimer's & Dementia, 7(3):270-279 (2011).
Andresen et al., “Effect of 5 days linaclotide on transit and bowel function in females with constipation-predominant irritable bowel syndrome,” Gastroenterology, 133:761-8 (2007).
Antonelou et al., “Decreased levels of alpha-synuclein in cerebrospinal fluid of patients with clinically isolated syndrome and multiple sclerosis,” J. Neurochem., 134(4):748-55 (2015).
Antonio-Rubio et al., “Abnormal thermography in Parkinson's disease,” Parkinsonism Relat. Disord., 21:852-7 (2015).
Auyeung et al., “Ten year survival and outcomes in a prospective cohort of new onset Chinese Parkinson's disease patients,” J. Neurol. Neurosurg. Psychiatry, 83:607-11 (2012).
Aziz et al. “Sleep and circadian rhythm alterations correlate with depression and cognitive impairment in Huntington's disease” Parkinsonism Relat Disord. 16(5):345-50 (2010).
Beghi et al., “The epidemiology of ALS and the role of population-based registries,” Biochim. Biophys. Acta, 1762: 1150-1157 (2006).
Berg et al., “MDS Research Criteria for Prodromal Parkinson's Disease,” Mov. Disord., 30:1600-1611 (2015).
Bhargava et al., “A phase I and pharmacokinetic study of squalamine, a novel antiangiogenic agent, in patients with advanced cancers,” Clin. Cancer Res., 7:3912-9 (2001).
Braak et al., “Idiopathic Parkinson's disease: possible routes by which vulnerable neuronal types may be subject to neuroinvasion by an unknown pathogen,” J. Neural. Transm. (Vienna), 110:517-36 (2003).
Braak et al., “Staging of brain pathology related to sporadic Parkinson's disease,” Neurobiol. Aging, 24:197-211 (2003).
Braak et al., “Gastric alpha-synuclein immunoreactive inclusions in Meissner's and Auerbach's plexuses in cases staged for Parkinson's disease-related brain pathology,” Neuroscience Letters, 396:67-72 (2006).
Breen et al., “Sleep and circadian rhythm regulation in early Parkinson disease,” JAMA Neurol., 71:589-95 (2014).
Breen, D. P. & Lang, A. E., “Tracking the Course of Prodromal Parkinson's Disease,” Brain, 140:259-262 (2017).
Brundin et al., “Therapeutic approaches to target alpha-synuclein pathology,” Exp. Neurology, 298(B):225-235 (2017).
Caudal et al. Experimental Neurology 273:243-252 (2015).
Chang et al., “A Meta-Analysis of Genome-Wide Association Studies Identifies 17 New Parkinson's Disease Risk Loci,” Nat. Genet., 49:1511-1516 (2017).
Cordell et al., “Alzheimer's Association Recommendations for Operationalizing the Detection of Cognitive Impairment During the Medicare Annual Wellness Visit in a Primary Care Setting,” Alzheimer's & Dementia, 9(2):141-150 (2013).
Corrochano et al., “α-synuclein levels affect autophagosome numbers in vivo and modulate Huntington disease pathology,” Autophagy, 8(3):431-432 (March 2012).
Cummings et al. “Depression in vascular dementia” Hillside J Clin Psychiatry 10(2):209-31(1988).
Darweesh et al., “Trajectories of Prediagnostic Functioning in Parkinson's Disease,” Brain, 140:429-441 (2017).
Demirel et al., “Decreased Expression of α-Synuclein, Nogo-A and UCH-L1 in Patients with Schizophrenia: A Preliminary Serum Study,” Psychiatry Invest., 14(3) (2017).
Diederich et al., “Hallucinations in Parkinson disease,” Nat. Rev. Neurol., 5:331-42 (2006).
Epping et al. “Depression in the early stages of Huntington's disease” Neurodegener Dis Manag. 2011 Oct. 1; 1(5): 407-414.
Esmonde et al. “Neuropsychological performance, disease severity, and depression in progressive supranuclear palsy” J Neurol 243(9):638-43 (1996).
Fahn S E R, Members of the UPDRS Development Committee. UNIFIED PARKINSON'S DISEASE RATING SCALE. Florham Park, N.J.: Macmillan Health Care Information (1987).
Frank et al., “Psychometric validation of a constipation symptom assessment questionnaire,” Scand. J. Gastroenterol., 34:870-7 (1999).
Friedman J H, Akbar U., “Psychosis in Parkinson's disease: unexplained observations in a seemingly simple model,” Expert Rev. of Neurotherapeutics, 16:595-6 (2016).
Frieling et al., “Alpha-synuclein mRNA levels correspond to beck depression inventory scores in females with eating disorders,” Neuropsychobiology, 58(1):48-52 (2008).
Frisina et al., “The neuropathological basis for depression in Parkinson's disease” Parkinsonism Relat Disord. 2009 February; 15(2): 144-148.
Gatchel et al. “Depressive symptoms and tau accumuliation in the inferior temporal lobe and entorhinal cortex in cognitively normal older adults: a pilor study” J Alzheimers Dis. 59(3):975-985 (2017).
Germain et al., “Circadian rhythm disturbances in depression” Hum. Psychopharmacol. 2008 October; 23(7): 571-585.
Gerstenecker et al. “Behavorial abnormalities in progressive supranuclear palsy” Psychiatry Res. 210(3) (2013).
Ghaziuddin et al. “Depression in Persons with Autism: Implications for Research and Clinical Care” Journal of Autism and Developmental Disorders 32(4):299-306 (2002).
Gjerstad et al., “Excessive daytime sleepiness in Parkinson disease: is it the drugs or the disease?” Neurology, 67:853-8 (2006).
Gozdzik-Zelazny et al., “Depressive symptoms in schizophrenic patients” Eur J Med Res. 16(12):549-552 (2011).
Goetz C G, Stebbins G T., “Risk factors for nursing home placement in advanced Parkinson's disease,” Neurology, 43:2227-9 (1993).
Hao et al., “A Phase I and pharmacokinetic study of squalamine, an aminosterol angiogenesis inhibitor,” Clin. Cancer Res., 9:2465-71 (2003).
Happe et al. “Sleep disorders and depression in paitents with Parkinson's disease” Act a Neurologica Scandinavica 104(5):275-280 (2008).
Heaton et al., “Defecation frequency and timing, and stool form in the general population: a prospective study,” Gut, 33:818-24 (1992).
Holmqvist et al., “Direct evidence of Parkinson pathology spread from the gastrointestinal tract to the brain in rats,” Acta Neuropathol., 128:805-20 (2014).
Hosokawa et al., “Accumulation of multiple neurodegenerative disease-related proteins in familial frontotemporal lobar degeneration associated with granulin mutation,” Scientific Reports, 7:1513 (2017).
Hughes et al., “Accuracy of clinical diagnosis of idiopathic Parkinson's disease: a clinico-pathological study of 100 cases,” J. Neurol. Neurosurg. Psychiatry, 55:181-4 (1992).
Hughes et al., “Associations of Probable REM Sleep Behavior Disorder, Constipation, and Hyposmia with PD” (2017), in Movement Disorder Society: Proceedings of the International Congress of Parkinson's Disease and Movement Disorders; Marsili et al., 2018. Diagnostic Criteria for Parkinson's Disease: From James Parkinson to the Concept of Prodromal Disease. Front. Neurol., Online 23 Mar. 2018.
Jack et al., “Introduction to the Recommendations from the National Institute on Aging—Alzheimer's Association Workgroups on Diagnostic Guidelines for Alzheimer's Disease,” Alzheimer's & Dementia, 7(3):257-262 (2011).
Jennings et al., “Hyposmic and Dopamine Transporter-Deficit Prodromal Cohort,” JAMA Neurol., 74:933-940 (2017).
Jorm, A. F., “The Informant Questionnaire on Cognitive Decline in the Elderly (IQCODE): A Review,” International Psychogeriatrics, 16:1-19 (2004).
Kim et al. “Major Depressive Disorder Preceding the Onset of Progressive Supranuclear Palsy” Psychiatry Investig. 6(2):112-114 (2009).
Kim et al. “Poststroke Induction of α-Synuclein Mediates Ischemic Brain Damage” J Neurosci, 2016 Jun. 29; 36(26):7055-65
Kirkevold, O. & Selbaek, G., “The Agreement Between the MMSE and IQCODE Tests in a Community-Based Sample of Subjects Aged 70 Years or Older Receiving In-Home Nursing: An Explorative Study,” Dement Geriatr. Cogn. Dis. Extra, 5(1):32-41 (2015).
Kho P O. “Cerebral ischemic injury decreases α-synuclein expression in brain tissue and glutamate-exposed HT22 cells,” Lab Anim Res. 33(3):244-250 (2017).
Kokubo et al., “α-Synuclein Pathology in the Amyotrophic Lateral Sclerosis/Parkinsonism Dementia Complex in the Kii Peninsula, Japan,” J. of Neuropathology & Exp. Neurology, 71(7):625-630 (2012).
Levy et al., “Alzheimer disease and frontotemporal dementias. Behavioral distinctions.” Arch Neurol. 1996; 53:687-690.
Lewis S J, Heaton K W., “Stool form scale as a useful guide to intestinal transit time,” Scand. J. Gastroenterol., 32:920-4 (1997).
Lin et al., “Risk of Parkinson's disease following severe constipation: a nationwide population-based cohort study,” Parkinsonism Relat. Disord., 20:1371-5 (2014).
Lu et al. “Association of alpha-synuclein immunoreactivity with inflammatory activity in multiple sclerosis lesions” J Neuropathol Exp Neurol. 68(2):179-89 (2009).
Madrid-Navarro et al., “Multidimensional Circadian Monitoring by Wearable Biosensors in Parkinson's Disease,” Front. Neurol., 9:157 (2018).
I. Magen and M. Chesselet, “Mouse Models of Cognitive Deficits Due to Alpha-Synuclein Pathology,” J. of Parkinson's Dis., 1:217-227 (2011).
Mahlknecht et al., “Prodromal Parkinson's Disease as Defined per MDS Research Criteria in the General Elderly Community,” Mov. Disord., 31:1405-1408 (2016).
Marquis et al., “Development and validation of the Patient Assessment of Constipation Quality of Life questionnaire,” Scand. J. Gastroenterol., 40:540-51 (2005).
Marsili et al., “Diagnostic Criteria for Parkinson's Disease: From James Parkinson to the Concept of Prodromal Disease,” Front. Neurol., Online 23 Mar. 2018.
McDowell et al., “Sleep dysfunction and EEG alterations in mice overexpressing alpha-synuclein,” J. Parkinsons Dis., 4(3):531-539 (2014).
McKhann et al., “The Diagnosis of Dementia Due to Alzheimer's Disease: Recommendations from the National Institute on Aging-Alzheimer's Association Workgroups on Diagnostic Guidelines for Alzheimer's Disease,” Alzheimer's & Dementia, 7(3):263-269 (2011).
Mearin et al., “Bowel Disorders,” Gastroenterology, 150(6):1393-1407 (2016).
Mejia et al., “Low Levels of Alpha-Synuclein in Peripheral Tissues Are Related to Clinical Relapse in Relapsing-Remitting Multiple Sclerosis (P1.392),” Neurology, 90(15 Supplement) (Apr. 10, 2018).
Menza et al. “Psychiatric Symptoms in Progressive Supranuclear Palsy” Psychosomatics 36(6):550-554 (1995).
Moussaud et al., “Alpha-synuclein and tau: teammates in neurodegeneration?” Mol. Neurodeg., 9:43 (2014).
S. Morairty, “Detecting Neurodegenerative Diseases Before Damage Is Done,” SRI International (Jul. 26, 2013) (https://www.sri.com/blog/detecting-neurodegenerative-diseases).
Morimoto et al. “Diagnosis and Treatment of Depression and Cognitive Impairment in Late-Life” Ann N Y Acad Sci. 1345(1):26-46 (2016).
Nemeroff et al. “Heatache and heartbreak—the link between depression and cardiovascular disease” Nature Reviews Cardiology 9:526-539 (2012).
Nutt et al. “Sleep disorders as core symptoms of depression” Dialogues Clin Neurosci. 10(3):329-336 (2008).
O'connor et al. “Depression and ischemic heart disease” Ann Heart J. 140(4) 63-9 (2000).
Ondo et al., “Daytime sleepiness and other sleep disorders in Parkinson's disease,” Neurology, 57:1392-6 (2001).
Ondo et al., “Placebo-controlled trial of lubiprostone for constipation associated with Parkinson disease,” Neurology, 78:1650-4 (2012).
Ortiz-Tudela et al., “Ambulatory circadian monitoring (ACM) based on thermometry, motor activity and body position (TAP): a comparison with polysomnography,” Physiol. Behav., 126:30-8 (2014).
Pagano, G., “Imaging in Parkinson's Disease,” Clin. Med., 16:371-375 (2016).
Palsetia et al., “The Clock Drawing Test versus Mini-mental Status Examination as a Screening Tool for Dementia: A Clinical Comparison,” Indian J. Psychol. Med., 40:1-10 (2018).
Papapetropoulos et al., “A questionnaire-based (UM-PDHQ) study of hallucinations in Parkinson's disease,” BMC Neurol., 8:21 (2008).
Perni et al., “A natural product inhibits the initiation of alpha-synuclein aggregation and suppresses its toxicity,” PNAS, USA, 114:E1009-E17 (2017).
Phillips et al., “Alpha-synuclein-immunopositive myenteric neurons and vagal preganglionic terminals: autonomic pathway implicated in Parkinson's disease?” Neuroscience, 153:733-50 (2008).
Phukan et al., “Cognitive impairment in amyotrophic lateral sclerosis,” Lancet Neurol., 6(11):994-1003 (2007).
Plassman et al., “Prevalance of Cognitive Impairment Without Dementia in the United States,” Ann. Intern. Med., 148(6):427-434 (2009).
Postuma et al., “The New Definition and Diagnostic Criteria of Parkinson's Disease,” Lancet Neurol., 15:546-548 (2016).
Postuma et al., “The New Definition and Diagnostic Criteria of Parkinson's Disease,” Lancet Neurol. 15:546-548 (2016).
Rahn et al., “Cognitive Impairment in Multiple Sclerosis: A Forgotten Disability Remembered,” Cerebrum., 201214 (November-December 2012).
Reeve et al., “Ageing and Parkinson's disease: why is advancing age the biggest risk factor?” Ageing Res. Rev., 14: 19-30 (2014).
Reitz et al., “Epidemiology of Alzheimer disease,” Nat. Rev. Neurol., 7: 137-152 (2011).
Rocca et al., “The Role of T1-Weighted Derived Measures of Neurodegeneration for Assessing Disability Progression in Multiple Sclerosis,” Front Neurol., 8:433 (Sep. 4, 2017).
Roos et al., “Depression in amyotrophic lateral sclerosis” Neurology. 2016 Jun. 14; 86(24):2271-7.
E. Rubin, “Cognitive Impairment in Depression,” Psychology Today (Jun. 8, 2016)
Sarabia et al., “Circadian rhythm of wrist temperature in normal-living subjects A candidate of new index of the circadian system,” Physiol. Behav., 95:570-80 (2008).
Shehata et al., “Neuronal stimulation induces autophagy in hippocampal neurons that is involved in AMPA receptor degradation after chemical long-term depression,” J. Neurosci., 32:10413-22 (2012).
Siegert et al. Siegert R J, Abernethy D A Depression in multiple sclerosis: a review Journal of Neurology, Neurosurgery & Psychiatry 76:469-475 (2005).
Silva et al. “Neuroanatomical correlates of depression in Friedreich's ataxia: a voxel-based morphometry study” Cerebellum 12(3):429-36 (2013).
Siris et al., “Depression in schizophrenia: recognition and management in the USA” Schizophrenia Research Volume 47, Issues 2-3, 1 Mar. 2001, Pages 185-197
Jon Stoessl, “Neuroimaging in the early diagnosis of neurodegenerative disease,” Transl. Neurodegener., 1: 5 (2012).
Steer et al., “Use of the Beck Depression Inventory-II with depressed geriatric inpatients,” Behav. Res. Ther., 38:311-8 (2000).
Stiasny-Kolster et al., “The REM sleep behavior disorder screening questionnaire—a new diagnostic instrument,” Movement disorders: Official J. of the Movement Dis. Soc., 22:2386-93 (2007).
Stolzenberg et al., “A Role for Neuronal Alpha-Synuclein in Gastrointestinal Immunity,” J. Innate Immun., 9:456-63 (2017).
Svensson et al., “Does vagotomy reduce the risk of Parkinson's disease: The authors reply” Ann. Neurol., 78:1012-3 (2015).
Sriwimol et al. “Significant Changes in Plasma Alpha-Synuclein and Beta-Synuclein Levels in Male Children with Autism Spectrum Disorder,” BioMed Research International, vol. 2018, Article ID 4503871, 7 pages, 2018
Tang et al., “Loss of mTOR-Dependent Macroautophagy Causes Autistic-like Synaptic Pruning Deficits,” Neuron, 83(5):1131-1143 (2014).
Tamo et al. “Expression of α-synuclein, the precursor of non-amyloid β component of Alzheimer's disease amyloid, in human cerebral blood vessels” Neuroscience Letters Volume 326, Issue 1, 21 Jun. 2002, Pages 5-8
Thomas, A J et al. “Depression and vascular disease: what is the relationship?” J Affect Disord. 2004 April; 79(1-3):81-95.
Vatagia et al. “Magnetic Resonance Imaging Correlates of Depression After Ischemic Stroke” Arch Gen Psychiatry 25(10):925-931 (2001).
Videnovic A, Golombek D., “Circadian Dysregulation in Parkinson's Disease,” Neurobiol. Sleep Circadian Rhythms, 2:53-8 (2017).
Wan et al., “α-Synuclein induced toxicity in brain stem serotonin neurons mediated by an AAV vector driven by the tryptophan hydroxylase promoter” Scientific Reports 6 26285 (2016).
West et al., “Squalamine increases vagal afferent firing frequency in aging mice,” J. of the Canadian Association of Gatroenterology, 1 (2018).
Wimo et al., “The worldwide economic impact of dementia 2010,” Alzheimer's Dement., 9: 1-11 (2013).
Yancopoulou et al., “Tau and alpha-synuclein inclusions in a case of familial frontotemporal dementia and progressive aphasia,” J. Neuropathol. Exp. Neurol., 64(3):245-53 (2005).
Zahodne et al., “Mood and motor trajectories in Parkinson's disease: multivariate latent growth curve modeling,” Neuropsychology, 26:71-80 (2012).
Zhang et al. “Depression and anxiety in multiple system atrophy.” Acta Neurol Scand. 2018 January; 137(1):33-37.
Zhao H Q, Li F F, Wang Z, Wang X M, Feng T. “A comparative study of the amount of α-synuclein in ischemic stroke and Parkinson's disease,” Neurol Sci. 37(5):749-54 (2016).
Zinsmeister et al., “Pharmacodynamic and clinical endpoints for functional colonic disorders: statistical considerations,” Dig. Dis. Sci., 58:509-18 (2013).
Learning About Parkinson's Disease, NIH Nat'l Human Genome Research Inst., https://www.genome.gov/10001217/learning-about-parkinsons-disease/(last updated Mar. 14, 2014); Parkinson Disease, NIH U.S. Nat'l Library of Med.: Genetics Home Reference, https://ghr.nlm.nih.gov/condition/parkinson-disease#genes (last updated Jul. 3, 2018).

What is claimed is:

1. A method of treating, preventing, and/or slowing the onset or progression of depression and/or a related symptom in a subject in need comprising administering to the subject a therapeutically effective amount of at least one aminosterol, or a salt or derivative thereof, provided that the administering does not comprise oral administration.

2. The method of claim 1, wherein administering comprises administration selected from nasal, sublingual, buccal, rectal, vaginal, intravenous, intra-arterial, intradermal, intraperitoneal, intrathecal, intramuscular, epidural, intracerebral, intracerebroventricular, transdermal, or any combination thereof.

3. The method of claim 1, wherein administering comprises nasal administration.

4. The method of claim 1, wherein the therapeutically effective amount of at least one aminosterol, or a salt or derivative thereof:
(a) comprises about 0.1 to about 20 mg/kg body weight of the subject; and/or
(b) comprises about 0.1 to about 15 mg/kg body weight of the subject; and/or
(c) comprises about 0.1 to about 10 mg/kg body weight of the subject; and/or
(d) comprises about 0.1 to about 5 mg/kg body weight of the subject; and/or
(e) comprises about 0.1 to about 2.5 mg/kg body weight of the subject; and/or
(f) comprises about 0.001 to about 500 mg/day; and/or
(g) comprises about 0.001 to about 250 mg/day; and/or
(h) comprises about 0.001 to about 125 mg/day; and/or
(i) comprises about 0.001 to about 50 mg/day; and/or
(j) comprises about 0.001 to about 25 mg/day; and/or
(k) comprises about 0.001 to about 10 mg/day; and/or
(l) comprises about 0.001 to about 6 mg/day administered intranasal; and/or
(m) comprises about 0.001 to about 4 mg/day administered intranasal; and/or
(n) comprises about 0.001 to about 2 mg/day administered intranasal; and/or
(o) comprises about 0.001 to about 1 mg/day administered intranasal; and/or
(p) comprises about 1 to about 300 mg/day administered orally; and/or
(q) comprises about 25 to about 300 mg/day administered orally.

5. The method of claim 1, wherein:
(a) the aminosterol or a salt or derivative thereof is taken on an empty stomach, optionally within two hours of the subject waking; and/or
(b) no food is taken or consumed after about 60 to about 90 minutes of taking the aminosterol or a salt or derivative thereof; and/or
(c) the aminosterol or a salt or derivative thereof is a pharmaceutically acceptable grade of at least one aminosterol or a pharmaceutically acceptable salt or derivative thereof; and/or
(d) the aminosterol is comprised in a composition further comprising one or more of the following: an aqueous carrier; a buffer; a sugar; and/or a polyol compound; and/or
(e) the subject is human; and/or
(f) the subject is a member of a patient population or an individual at risk for developing depression.

6. The method of claim 1, wherein the aminosterol or the salt or derivative thereof is:
(a) isolated from the liver of Squalus acanthias; and/or
(b) squalamine or a pharmaceutically acceptable salt thereof; and/or
(c) a squalamine isomer; and/or
(d) the phosphate salt of squalamine; and/or
(e) aminosterol 1436 or a pharmaceutically acceptable salt thereof; and/or
(f) an isomer of aminosterol 1436; and/or
(g) the phosphate salt of aminosterol 1436; and/or
(h) comprises a sterol nucleus and a polyamine attached at any position on the sterol, such that the molecule exhibits a net charge of at least +1; and/or
(i) comprises a bile acid nucleus and a polyamine, attached at any position on the bile acid, such that the molecule exhibits a net charge of at least +1; and/or
(j) a derivative modified to include one or more of the following:
(i) substitutions of the sulfate by a sulfonate, phosphate, carboxylate, or other anionic moiety chosen to circumvent metabolic removal of the sulfate moiety and oxidation of the cholesterol side chain;
(ii) replacement of a hydroxyl group by a non-metabolizable polar substituent, such as a fluorine atom, to prevent its metabolic oxidation or conjugation; and
(iii) substitution of one or more ring hydrogen atoms to prevent oxidative or reductive metabolism of the steroid ring system; and/or
(k) a derivative of squalamine modified through medicinal chemistry to improve bio-distribution, ease of administration, metabolic stability, or any combination thereof; and/or
(l) a synthetic aminosterol; and/or
(m) is selected from the group consisting of:

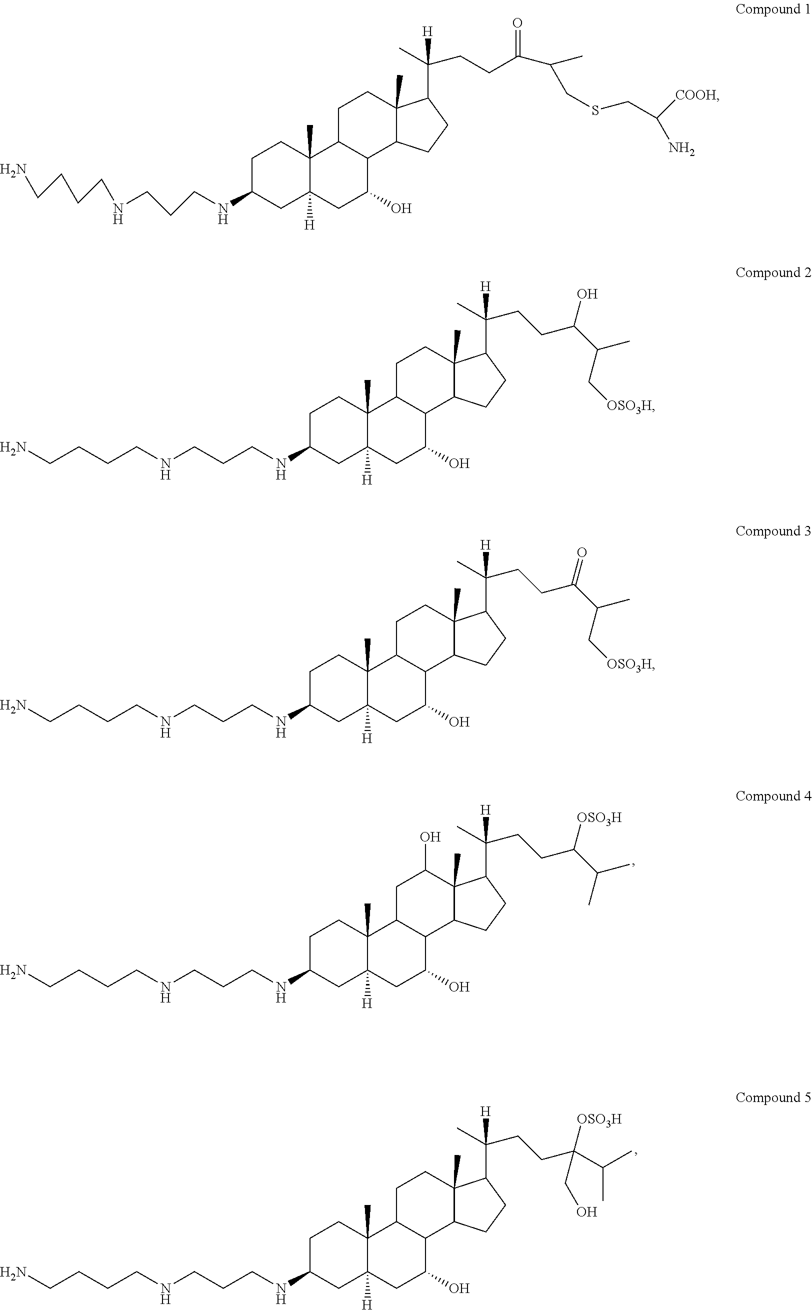

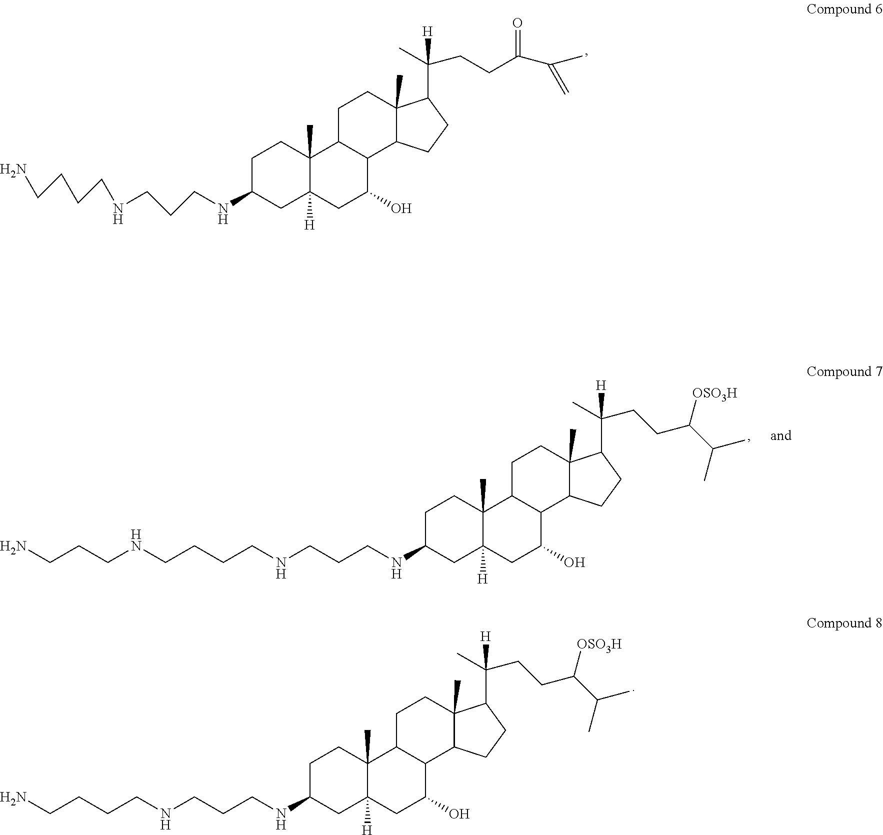

7. A method of treating, preventing, and/or slowing the onset or progression of depression and/or a related symptom in a subject in need comprising:
(a) determining a dose of an aminosterol or a salt or derivative thereof for the subject, wherein the aminosterol dose is determined based on the effectiveness of the aminosterol dose in improving or resolving a depression symptom being evaluated,
(b) followed by administering the dose of the aminosterol or a salt or derivative thereof to the subject for a defined period of time, wherein the method comprises:
(i) identifying a depression symptom to be evaluated;
(ii) identifying a starting dose of an aminosterol or a salt or derivative thereof for the subject; and
(iii) administering an escalating dose of the aminosterol or a salt or derivative thereof to the subject over a defined period of time until an effective dose for the depression symptom being evaluated is identified, wherein the effective dose is the aminosterol dose where improvement or resolution of the depression symptom is observed, and fixing the aminosterol dose at that level for that particular depression symptom in that particular subject; and
(c) optionally wherein each defined period of time is independently selected from the group consisting of about 1 day to about 10 days, about 10 days to about 30 days, about 30 days to about 3 months, about 3 months to about 6 months, about 6 months to about 12 months, and about greater than 12 months.

8. The method of claim 7, wherein the amino sterol or a salt or derivative thereof is administered orally, intranasally, or a combination thereof.

9. The method of claim 8, wherein the amino sterol or a salt or derivative thereof is administered orally and:
(a) the starting dose of the aminosterol or a salt or derivative thereof ranges from about 1 mg up to about 175 mg/day; and/or
(b) the starting oral aminosterol dose is about 25 mg/day; and/or
(c) the dose of the aminosterol or a salt or derivative thereof for the subject following escalation is fixed at a range of from about 1 mg up to about 500 mg/day; and/or
(d) the dose of the aminosterol or a salt or derivative thereof for the subject following escalation is fixed at a dose of about 1, about 5, about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, about 205, about 210, about 215, about 220, about 225, about 230, about 235, about 240, about 245, about 250, about 255, about 260, about 265, about 270, about 275, about 280, about 285, about 290, about 295, about 300, about 305, about 310, about 315, about 320, about 325, about 330, about 335, about 340, about 345, about 350, about 355, about 360, about 365, about 370, about 375, about 380, about 385, about 390, about 395, about 400, about 405, about 410, about 415, about 420, about 425, about 430, about 435, about 440, about 445, about 450, about 455, about 460, about 465, about 470, about 475, about 480, about 485, about 490, about 495, or about 500 mg/day; and/or
(e) the starting oral aminosterol dose is about 10, about 15, about 20, about 25, about 30, about 35, about 40, about 45, about 60, about 65, about 70, or about 75 mg/day; and/or
(f) the dose of the aminosterol or a salt or derivative thereof is escalated in about 25 mg increments; and/or
(g) the aminosterol or a salt or derivative thereof is formulated for oral administration in a composition which is a liquid, capsule, or tablet designed to disintegrate in either the stomach, upper small intestine, or more distal portions of the intestine.

10. The method of claim 8, wherein the aminosterol or a salt or derivative thereof is administered intranasally and:
(a) the starting dose of the aminosterol or a salt or derivative thereof ranges from about 0.001 mg to about 3 mg/day; and/or
(b) the dose of the aminosterol or a salt or derivative thereof for the subject following escalation is fixed at a range of from about 0.001 mg up to about 6 mg/day; and/or
(c) the dose of the aminosterol or a salt or derivative thereof for the subject following escalation is a dose which is subtherapeutic when administered orally or by injection; and/or
(d) the dose of the aminosterol or a salt or derivative thereof is escalated in increments of about 0.1, about 0.2, about 0.25, about 0.3, about 0.35, about 0.4, about 0.45, about 0.5, about 0.55, about 0.6, about 0.65, about 0.7, about 0.75, about 0.8, about 0.85, about 0.9, about 0.95, about 1, about 1.1, about 1.2, about 1.3, about 1.4, about 1.5, about 1.6, about 1.7, about 1.8, about 1.9, or about 2 mg; and/or
(e) the aminosterol or a salt or derivative thereof is formulated for intranasal administration in a composition which is a dry powder nasal spray or liquid nasal spray.

11. The method of claim 7, wherein:
(a) the dose of the aminosterol or a salt or derivative thereof is escalated every about 3 to about 5 days; and/or
(b) the dose of the aminosterol or a salt or derivative thereof is escalated every about 1 to about 14 days; and/or
(c) the dose of the aminosterol or a salt or derivative thereof is escalated every about 1, about 2, about 3, about 4, about 5, about 6, about 7, about 8, about 9, about 10, about 11, about 12, about 13, or about 14 days; and/or
(d) the dose of the aminosterol or a salt or derivative thereof is escalated about 1×/week, about 2×/week, about every other week, or about 1×/month; and/or
(e) the fixed dose of the aminosterol or a pharmaceutically acceptable salt or derivative thereof is administered once per day, every other day, once per week, twice per week, three times per week, four times per week, five times per week, six times per week, every other week, or every few days; and/or
(f) the fixed dose of the aminosterol or a pharmaceutically acceptable salt or derivative thereof is administered for a first defined period of time of administration, followed by a cessation of administration for a second defined period of time, followed by resuming administration upon recurrence of depression or a symptom of depression; and/or
(g) the fixed aminosterol dose is incrementally reduced after the fixed dose of aminosterol or a pharmaceutically acceptable salt or derivative thereof has been administered to the subject for a period of time; and/or
(h) the fixed aminosterol dose is varied plus or minus a defined amount to enable a modest reduction or increase in the fixed dose; and/or
(i) the fixed aminosterol dose is varied plus or minus a defined amount to enable a modest reduction or increase in the fixed dose, and the fixed aminosterol dose is increased or decreased by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20%; and/or
(j) the starting dose of the aminosterol or a pharmaceutically acceptable salt or derivative thereof is higher if the symptom being evaluated is severe; and/or
(k) each defined period of time is independently selected from the group consisting of about 1 day to about 10 days, about 10 days to about 30 days, about 30 days to about 3 months, about 3 months to about 6 months, about 6 months to about 12 months, and about greater than 12 months.

12. The method of claim 7, wherein:
(a) severity of the depression is reduced over a defined period of time, wherein the reduction is measured from one or more medically-recognized techniques selected from the group consisting of the Patient Health Questionnaire-9 (PHQ-9); the Beck Depression Inventory (BDI); Zung Self-Rating Depression Scale; Center for Epidemiologic Studies-Depression Scale (CES-D); and the Hamilton Rating Scale for Depression (HRSD); and/or
(b) progression or onset of depression is slowed, halted, or reversed over a defined period of time following administration of the fixed escalated dose of the aminosterol or a salt or derivative thereof, as measured by a medically-recognized technique; and/or
(c) the depression is positively impacted by the fixed escalated dose of the aminosterol or a salt or derivative thereof, as measured by a medically-recognized technique; and/or
(d) the positive impact on and/or progression of depression is measured quantitatively or qualitatively by one or more techniques selected from the group consisting of electroencephalogram (EEG), neuroimaging, functional MRI, structural MRI, diffusion tensor imaging (DTI), [18F]fluorodeoxyglucose (FDG) PET, agents that label amyloid, [18F]F-dopa PET, radiotracer imaging, volumetric analysis of regional tissue loss, specific imaging markers of abnormal protein deposition, multimodal imaging, and biomarker analysis; and/or
(e) the progression or onset of depression is slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique; and/or
(f) the fixed escalated aminosterol dose reverses dysfunction caused by the depression and treats, prevents, improves, and/or resolves the symptom being evaluated; and/or
(g) the improvement or resolution of the depression symptom is measured using a clinically recognized scale or tool; and/or
(h) the improvement in the depression symptom is at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, as measured using a clinically recognized scale; and/or
(i) the defined period of time wherein the severity of the depression is reduced is about 1 day to about 10 days, about 10 days to about 30 days, about 30 days to about 3 months, about 3 months to about 6 months, about 6 months to about 12 months, or about greater than 12 months.

13. The method of claim 7, wherein the depression symptom to be evaluated comprises a symptom selected from the group consisting of:
(a) a symptom from the Hamilton Depression Rating Scale (HAM-D) selected from the group consisting of depressed mood, feelings of guilt, suicide, initial insomnia, middle of night insomnia, delayed insomnia, work and interests, retardation, agitation, psychic anxiety, somatic anxiety, gastrointestinal symptoms, general somatic symptoms, genital symptoms, hypochondriasis, weight loss, insight, diurnal variation, depersonalization and derealization, paranoid symptoms, and obsessional symptoms;
(b) a symptom from the Montgomery-Asberg Depression Scale (MADRS) selected from the group consisting of apparent sadness, reported sadness, inner tension, reduced sleep, concentration difficulties, lassitude, inability to feel, pessimistic thoughts, and suicidal thoughts;
(c) a symptom from Beck's Depression Inventory (BDI) selected from the group consisting of sadness, outlook on the future, feelings of failure, satisfaction, guilt, feelings of being punished, disappointment with self, self-blame, suicidal ideation, crying frequency, prevalence of irritation, interest in others, ease of decision-making, self-image, ability to work, ease of sleep, tiredness, appetite, weight loss, preoccupation with health, and lack of libido;
(d) apathy;
(e) hopelessness;
(f) loss of interest in hobbies;
(g) sleep problem, sleep disorder, or sleep disturbance;
(h) excessive hunger;
(i) lack of appetite;
(j) restlessness;
(k) social isolation;
(l) cognitive impairment;
(m) weight loss;
(n) weight gain; and
(o) constipation.

14. The method of claim 13, wherein the depression symptom to be evaluated comprises a sleep problem, sleep disorder, sleep disturbance, circadian rhythm dysfunction, REM disturbed sleep, or REM behavior disorder, and wherein:
(a) the sleep problem, sleep disorder, or sleep disturbance comprises a delay in sleep onset, sleep fragmentation, REM-behavior disorder, sleep-disordered breathing including snoring and apnea, day-time sleepiness, micro-sleep episodes, narcolepsy, early awakening, insomnia, hallucinations, or any combination thereof; and/or
(b) the REM-behavior disorder comprises vivid dreams, nightmares, and acting out the dreams by speaking or screaming, or fidgeting or thrashing of arms or legs during sleep; and/or
(c) the method results in a positive change in the sleeping pattern of the subject over a defined period of time; and/or
(d) the method results in a positive change in the sleeping pattern of the subject over a defined period of time, wherein the positive change is defined as:
(i) an increase in the total amount of sleep obtained of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or
(ii) a percent decrease in the number of awakenings during the night selected from the group consisting of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%; and/or
(c) as a result of the method the subject obtains the total number of hours of sleep recommended by a medical authority for the age group of the subject; and/or
(d) each defined period of time is independently selected from the group consisting of about 1 day to about 10 days, about 10 days to about 30 days, about 30 days to about 3 months, about 3 months to about 6 months, about 6 months to about 12 months, and about greater than 12 months.

15. The method of claim 13, wherein the depression symptom to be evaluated comprises suicidal thoughts and wherein:
(a) the method results in a decreased number or severity of suicidal thoughts of the subject; and/or
(b) the method results in a decreased number or severity of suicidal thoughts of the subject over a defined period of time and the decrease in number or severity in suicidal thoughts is defined as a reduction in occurrences or severity of suicidal thoughts selected from the group consisting of by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or
(c) the method results in the subject being free of suicidal thoughts; and/or
(d) each defined period of time is independently selected from the group consisting of about 1 day to about 10 days, about 10 days to about 30 days, about 30 days to about 3 months, about 3 months to about 6 months, about 6 months to about 12 months, and about greater than 12 months.

16. The method of claim 13, wherein the depression symptom to be evaluated is sadness and wherein:
(a) the method results in improvement in the subject's sadness, as measured by one or more clinically-recognized depression rating scale; and/or
(b) the method results in improvement in the subject's sadness over a defined period of time, as measured by one or more clinically-recognized depression rating scale, and the improvement a subject experiences following treatment is about 5, about 10, about 15, about 20, about 25, about 30, about 35, about 40, about 45, about 50, about 55, about 60, about 65, about 70, about 75, about 80, about 85, about 90, about 95 or about 100%; and/or
(c) each defined period of time is independently selected from the group consisting of about 1 day to about 10 days, about 10 days to about 30 days, about 30 days to about 3 months, about 3 months to about 6 months, about 6 months to about 12 months, and about greater than 12 months.

17. The method of claim 13, wherein the depression symptom to be evaluated comprises cognitive impairment, and wherein:
(a) progression or onset of the cognitive impairment is slowed, halted, or reversed over a defined period of time following administration of the fixed escalated dose of the aminosterol or a salt or derivative thereof, as measured by a medically-recognized technique; and/or
(b) the cognitive impairment is positively impacted by the fixed escalated dose of the aminosterol or a salt or derivative thereof, as measured by a medically-recognized technique; and/or
(c) the cognitive impairment is positively impacted by the fixed escalated dose of the aminosterol or a salt or derivative thereof, as measured by a medically-recognized technique and the positive impact on and/or progression of cognitive impairment is measured quantitatively or qualitatively by one or more techniques selected from the group consisting of ADASCog, Mini-Mental State Exam (MMSE), Mini-cog test, Woodcock-Johnson Tests of Cognitive Abilities, Leiter International Performance Scale, Miller Analogies Test, Raven's Progressive Matrices, Wonderlic Personnel Test, IQ tests, and a computerized tested selected from Cantab Mobile, Cognigram, Cognivue, Cognision, or Automated Neuropsychological Assessment Metrics Cognitive Performance Test (CPT); and/or
(d) the progression or onset of cognitive impairment is slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique; and/or
(e) each defined period of time is independently selected from the group consisting of about 1 day to about 10 days, about 10 days to about 30 days, about 30 days to about 3 months, about 3 months to about 6 months, about 6 months to about 12 months, and about greater than 12 months.

18. The method of claim 13, wherein the depression symptom to be evaluated comprises constipation, and wherein:
(a) the fixed escalated aminosterol dose causes the subject to have a bowel movement; and/or
(b) the method results in an increase in the frequency of bowel movement in the subject; and/or
(c) the method results in an increase in the frequency of bowel movement in the subject over a defined period of time and the increase in the frequency of bowel movement is defined as:
(i) an increase in the number of bowel movements per week of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or
(ii) a percent decrease in the amount of time between each successive bowel movement selected from the group consisting of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%; and/or
(d) as a result of the method the subject has the frequency of bowel movement recommended by a medical authority for the age group of the subject; and/or
(e) the starting aminosterol dose is determined by the severity of the constipation, wherein:
(i) if the average complete spontaneous bowel movement (CSBM) or spontaneous bowel movement (SBM) is one or less per week, then the starting aminosterol dose is at least about 150 mg; and
(ii) if the average CSBM or SBM is greater than one per week, then the starting aminosterol dose is about 75 mg or less; and/or
(f) each defined period of time is independently selected from the group consisting of about 1 day to about 10 days, about 10 days to about 30 days, about 30 days to about 3 months, about 3 months to about 6 months, about 6 months to about 12 months, and about greater than 12 months.

19. The method of claim 13, wherein the depression symptom to be evaluated comprises lack of libido, and wherein:
(a) the method results in treating, preventing, and/or delaying the progression and/or onset of lack of libido in the subject; and/or
(b) progression or onset of the lack of libido is slowed, halted, or reversed over a defined period of time following administration of the fixed escalated dose of the aminosterol or a salt or derivative thereof, as measured by a medically-recognized technique; and/or
(c) the lack of libido is positively impacted by the fixed escalated dose of the aminosterol or a salt or derivative thereof, as measured by a medically-recognized technique; and/or
(d) the progression of (b) and/or the positive impact of (c) is measured quantitatively or qualitatively by one or more techniques selected from the group consisting of the Sexual Desire Inventory-2 (SDI-2), Brief Index for SF Form Women, Brief Sexual Function Questionnaire for Men, Deragotis Sexual Function Inventory (DSFI), Derogatis interview for Sexual Function, Female Sexual Arousability Index, Florida Sexual History Questionnaire (FSHQ), General Information Form (GIF), Golombok Rust Inventory of Sexual Satisfaction (GRISS), Hanson Assessment of Sexual Health, Heterosexual Behavior Assessment Females, Heterosexual Behavior Assessment Males, Heterosexual Zuckerman, Homosexual Zuckerman, Hypogonadism and Sexual Function, Index of Sexual Satisfaction (ISS), International Index of Erectile Function, Jewish General Hospital Sexual Self-Monitoring Form, Leiden Impotence Questionnaire, McCoy Female Sexuality Questionnaire, Multiaxial Problem-oriented Diagnostic System of SF, Potency and Prostatectomy, Radical Prostatectomy Questionnaire, Sabbastberg Sexual Rating Scale (revised), Scalability of Sexual Experience, Segraves Sexual Symptomatology Interview, Sexual Activity of Men presenting Prostatism and Prostatectomy, Sexual Adjustment Questionnaire (SAQ), Sexual Dysfunction (Silence Hurts), Sexual Dysfunction in HIV+Men (assoc w/neuropathy/CD4 count), Sexual Dysfunction in HIV+Men, Sexual Dysfunction in Schizophrenic Patients, Sexual Function Scale, Sexual Interaction Inventory (SII), Sexual Interaction System Scale, Sexual Interest and Satisfaction Scale, Sexual Interest Questionnaire (SIQ), Sexual Inventory (SI), Sexual Orientation Method and Anxiety (SOMA), Sexual Self-Efficacy Scale for Erectile Disorder (SSES-E), Sexual Symptom Distress Scale, Sexuality Experience Scale, The Clark Sexual History Questionnaire, Urge-incontinence Impact Questionnaire, Vaginal Changes and Sexuality in Women with Cervical CA, and Watts Sexual Function Questionnaire; and/or
(e) the progression or onset of (b) is slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique; and/or
(f) each defined period of time is independently selected from the group consisting of about 1 day to about 10 days, about 10 days to about 30 days, about 30 days to about 3 months, about 3 months to about 6 months, about 6 months to about 12 months, and about greater than 12 months.

20. The method of claim 7, wherein:
(a) the aminosterol or a salt or derivative thereof is administered in combination with at least one additional active agent to achieve either an additive or synergistic effect; and/or
(b) the additional active agent is administered via a method selected from the group consisting of concomitantly; as an admixture; separately and simultaneously or concurrently; and separately and sequentially; and/or
(c) the additional active agent is a different aminosterol from that administered in the method of claim 7;
(d) the method of claim 7 comprises a first aminosterol which is aminosterol 1436 or a salt or derivative thereof administered intranasally and a second aminosterol which is squalamine or a salt or derivative thereof administered orally; and/or
(e) the additional active agent is an active agent used to treat depression or a symptom thereof; and/or
(f) the additional active agent is an active agent used to treat depression or a symptom thereof and wherein the active agent is selected from the group consisting of selective serotonin reuptake inhibitors (SSRIs) such as citalopram (Celexa®, Cipramil®), escitalopram (Lexapro®, Cipralex®), paroxetine (Paxil®, Seroxat®), fluoxetine (Prozac®), fluvoxamine (Luvox®, Faverin®), sertraline (Zoloft®, Lustral®), indalpine (Upstene®), zimelidine (Normud®, Zelmid®); serotonin-norepinephrine reuptake inhibitors (SNRIs) such as desvenlafaxine (Pristiq®), duloxetine (Cymbalta®), levomilnacipran (Fetzima®), milnacipran (Ixel®, Savella®), venlafaxine (Effexor®); serotonin modulators and stimulators (SMSs) such as vilazodone (Viibryd®), vortioxetine (Trintellix®); serotonin antagonists and reuptake inhibitors such as nefazodone (Dutonin®, Nefadar®, Serzone®), trazodone (Desyrel®), etoperidone; norepinephrine reuptake inhibitors (NRIs) such as reboxetine (Edronax®), teniloxazine (Lucelan®, Metatone®), viloxazine (Vivalan®), atomoxetine (Strattera®); norepinephrine-dopamine reuptake inhibitors such as bupropion (Wellbutrin®), amineptine (Survector®, Maneon®), nomifensine (Merital®, Alival®), methylphenidate (Ritalin®, Concerta®), lisdexamfetamine (Vyvanse®); tricyclic antidepressants such asamitriptyline (Elavil®, Endep®), amitriptylinoxide (Amioxid®, Ambivalon®, Equilibrin®), clomipramine (Anafranil®), desipramine (Norpramin®, Pertofrane®), dibenzepin (Noveril®, Victoril®), dimetacrine (Istonil®), dosulepin (Prothiaden®), doxepin (Adapin®, Sinequan®), imipramine (Tofranil®), lofepramine (Lomont®, Gamanil®), melitracen (Dixeran®, Melixeran®, Trausabun®), nitroxazepine (Sintamil®), nortriptyline (Pamelor®, Aventyl®), noxiptiline (Agedal®, Elronon®, Nogedal®), opipramol (Insidon®), pipofezine (Azafen®/Azaphen®), protriptyline (Vivactil®), trimipramine (Surmontil®), butriptyline (Evadyne®), demexiptiline (Deparon®, Tinoran®), fluacizine (Phtorazisin®), imipraminoxide (Imiprex®, Elepsin®), iprindole (Prondol®, Galatur®, Tertran®), metapramine (Timaxel®), propizepine (Depressin®, Vagran®), quinupramine (Kinupril®, Kevopril®), tiazesim (Altinil®), tofenacin (Elamol®, Tofacine®), amineptine (Survector®, Maneon®), tianeptine (Stablon®, Coaxil®); tetracyclic antidepressants such as amoxapine (Asendin®), maprotiline (Ludiomil®), mianserin (Bolvidon®, Norval®, Tolvon®), mirtazapine (Remeron®), setiptiline (Tecipul®), mianserin, mirtazapine, setiptiline; monoamine oxidase inhibitors (MAOIs) such as isocarboxazid (Marplan®), phenelzine (Nardil®), tranylcypromine (Parnate®), benmoxin (Neuralex®), iproclozide (Sursum®), iproniazid (Marsilid®), mebanazine (Actomol®), nialamide (Niamid®), octamoxin (Ximaol®), pheniprazine (Catron®), phenoxypropazine (Drazine®), pivhydrazine (Tersavid®), safrazine (Safra®), selegiline (Eldepryl®, Zelapar®, Emsam®), caroxazone (Surodil®, Timostenil®), metralindole (Inkazan®), moclobemide (Aurorix®, Manerix®), pirlindole (Pirazidol®), toloxatone (Humoryl®), eprobemide (Befol®), minaprine (Brantur®, Cantor®), bifemelane (Alnert®, Celeport®); atypical antipsychotics such as amisulpride (Solian®), lurasidone (Latuda®), quetiapine (Seroquel®); or N-methyl D-aspartate (NMDA) antagonists such ketamine (Ketalar®); and/or
(f) the aminosterol or a salt or derivative thereof is taken on an empty stomach, optionally within two hours of the subject waking; and/or
(g) no food is taken after about 60 to about 90 minutes of taking the aminosterol or a salt or derivative thereof; and/or
(h) the aminosterol or a salt or derivative thereof is a pharmaceutically acceptable grade of at least one aminosterol or a pharmaceutically acceptable salt or derivative thereof; and/or
(i) the aminosterol or a salt or derivative thereof is comprised in a composition further comprising one or more of the following: an aqueous carrier; a buffer; a sugar; and/or a polyol compound; and/or
(j) the subject is a human; and/or
(k) the subject is a member of a patient population or an individual at risk for developing depression.

21. The method of claim 7, wherein the amino sterol or a salt or derivative thereof is:
(a) isolated from the liver of Squalus acanthias; and/or
(b) squalamine or a pharmaceutically acceptable salt thereof; and/or
(c) a squalamine isomer; and/or
(d) the phosphate salt of squalamine; and/or
(e) aminosterol 1436 or a pharmaceutically acceptable salt thereof; and/or
(f) an isomer of aminosterol 1436; and/or
(g) the phosphate salt of aminosterol 1436; and/or
(h) comprises a sterol nucleus and a polyamine attached at any position on the sterol, such that the molecule exhibits a net charge of at least +1; and/or
(i) comprises a bile acid nucleus and a polyamine, attached at any position on the bile acid, such that the molecule exhibits a net charge of at least +1; and/or
(j) a derivative modified to include one or more of the following:
(i) substitutions of the sulfate by a sulfonate, phosphate, carboxylate, or other anionic moiety chosen to circumvent metabolic removal of the sulfate moiety and oxidation of the cholesterol side chain;
(ii) replacement of a hydroxyl group by a non-metabolizable polar substituent, such as a fluorine atom, to prevent its metabolic oxidation or conjugation; and
(iii) substitution of one or more ring hydrogen atoms to prevent oxidative or reductive metabolism of the steroid ring system; and/or
(k) a derivative of squalamine modified through medicinal chemistry to improve bio-distribution, ease of administration, metabolic stability, or any combination thereof; and/or
(l) a synthetic aminosterol; and/or
(m) is selected from the group consisting of: 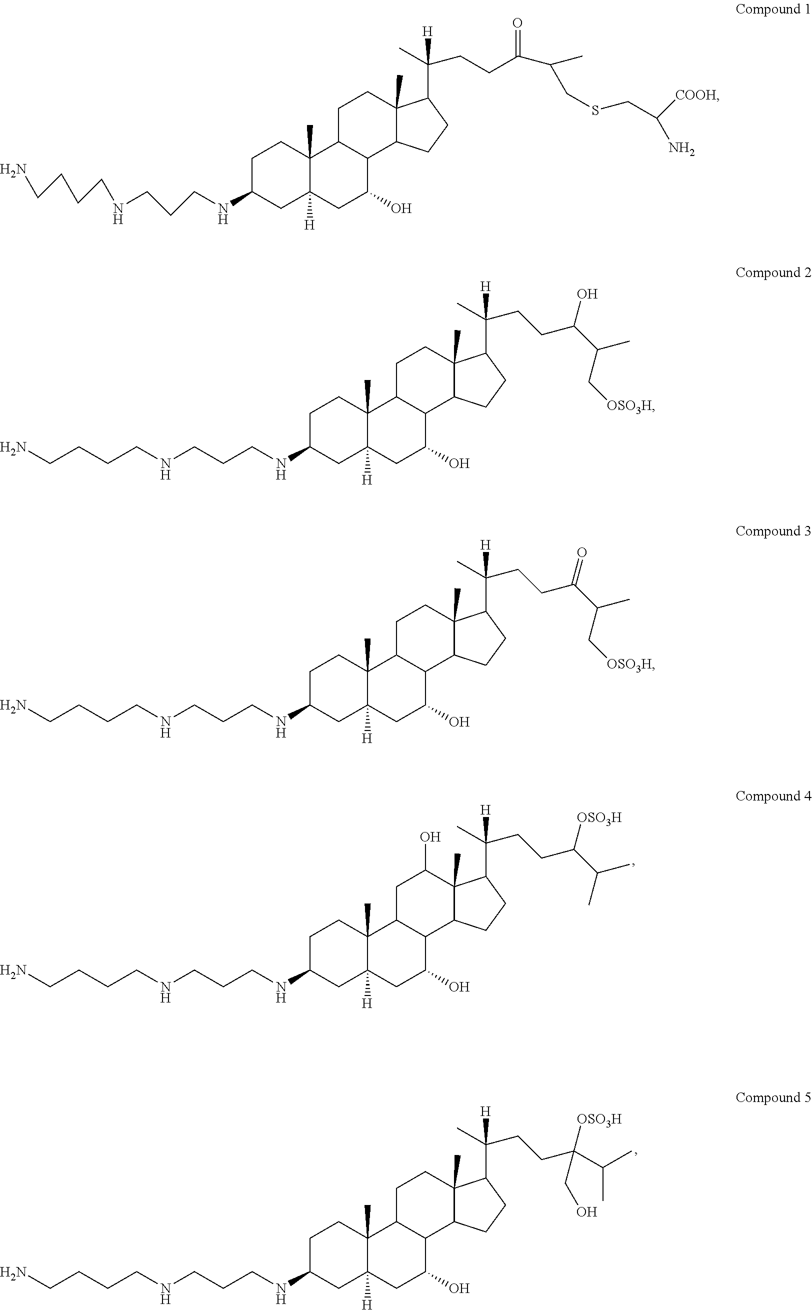 [chemical structure]
